(19) United States
(12) Patent Application Publication
(10) Pub. No.: US 2023/0098497 A1
(43) Pub. Date: Mar. 30, 2023

(54) Axial Insertion and Movement Along a Partially Constrained Path for Robotic Catheters and Other Uses

(71) Applicant: Project Moray, Inc., Belmont, CA (US)

(72) Inventor: Keith Phillip Laby, Oakland, CA (US)

(73) Assignee: Project Moray, Inc., Belmont, CA (US)

(21) Appl. No.: 18/072,398

(22) Filed: Nov. 30, 2022

Related U.S. Application Data
(63) Continuation of application No. PCT/US2021/037923, filed on Jun. 17, 2021.
(60) Provisional application No. 63/040,429, filed on Jun. 17, 2020.

(51) Int. Cl. A61B 34/37; A61B 34/10; A61B 90/00; A61B 34/00

(52) U.S. Cl. CPC A61B 34/37; A61B 34/10; A61B 90/37; A61B 34/25; A61B 2034/107; A61B 2034/102; A61B 2034/301

(57) ABSTRACT

Devices, systems, and methods are provided for control over automated movement of catheters and other elongate bodies. Fluid and/or pull-wire drive systems can be used to provide robotically coordinated lateral bending motions and a processor of the system can generate synchronized actuator drive signals to move the tool along an at least partially laterally constrained path, with the path optionally extending along the axis of a virtual model of the catheter that has been driven in silico into alignment with a target tissue adjacent an open workspace such as an open chamber of the heart.

### CROSS REFERENCE TO RELATED APPLICATIONS

This application is a Continuation of PCT/US2021/037923 filed Jun. 17, 2021; which claims the benefit of U.S. Provisional Appln No. 63/040,429 filed Jun. 17, 2020. The full disclosures which are incorporated herein by reference in their entirety for all purposes.

The subject matter of the present application is related to PCT/US2019/065752 filed Dec. 11, 2019 (published as WO 2020/123671). The full disclosure which is also incorporated herein by reference in its entirety for all purposes.

### FIELD OF THE INVENTION

In general, the present invention provides improved devices, systems, and methods for using, training for the use of, planning for the use of, and/or simulating the use of elongate articulate bodies and other tools such as catheters, borescopes, continuum robotic manipulators, rigid endoscopic robotic manipulators, and the like. In exemplary embodiments, the invention provides in situ robotic catheter motion planning, and catheter position control over complex trajectories, particularly for catheter movement advancing from along a tortuous and laterally constrained path and into an open chamber of the heart or other open workspace.

### BACKGROUND OF THE INVENTION

Diagnosing and treating disease often involve accessing internal tissues of the human body, and open surgery is often the most straightforward approach for gaining access to internal tissues. Although open surgical techniques have been highly successful, they can impose significant trauma to collateral tissues.

To help avoid the trauma associated with open surgery, a number of minimally invasive surgical access and treatment technologies have been developed, including elongate flexible catheter structures that can be advanced along the network of blood vessel lumens extending throughout the body. While generally limiting trauma to the patient, catheter-based endoluminal therapies can be very challenging, in-part due to the difficulty in accessing (and aligning with) a target tissue using an instrument traversing tortuous vasculature. Alternative minimally invasive surgical technologies include robotic surgery, and robotic systems for manipulation of flexible catheter bodies from outside the patient have also previously been proposed. Some of those prior robotic catheter systems have met with challenges, possibly because of the difficulties in effectively integrating large and complex robotic pull-wire catheter systems into the practice of interventional cardiology as it is currently performed in clinical catheter labs. While the potential improvements to surgical accuracy make these efforts alluring, the capital equipment costs and overall burden to the healthcare system of these large, specialized systems is also a concern. Examples of prior robotic disadvantages that would be beneficial to avoid may include longer setup and overall procedure times, deleterious changes in operative modality (such as a decrease in effective tactile feedback when initially accessing or advancing tools toward an internal treatment site), and the like.

A new technology for controlling the shape of catheters has recently been proposed which may present significant advantages over pull-wires and other known catheter articulation systems. As more fully explained in U.S. Pat. No. 10,646,696, entitled “Articulation Systems, Devices, and Methods for Catheters and Other Uses,” issued on May 12, 2020 (assigned to the assignee of the subject application and the full disclosure of which is incorporated herein by reference), an articulation balloon array can include subsets of balloons that can be inflated to selectively bend, elongate, or stiffen segments of a catheter. These articulation systems can direct pressure from a simple fluid source (such as a pre-pressurized canister) toward a subset of articulation balloons disposed along segment(s) of the catheter inside the patient so as to induce a desired change in shape. These new technologies may provide catheter control beyond what was previously available, often without having to resort to a complex robotic gantry, without having to rely on pull-wires, and even without having the expense of electric motors. Hence, these new fluid-driven catheter systems appear to provide significant advantages.

Along with the advantages of fluid-driven technologies, significant work is now underway on improved imaging for use by interventional and other doctors in guiding the movement of articulated therapy delivery systems within a patient. Ultrasound and fluoroscopy systems often acquire planar images (in some cases on different planes at angularly offset orientations), and news three-dimensional (3D) imaging technologies have been (and are still being) developed and used to show these 3D images. While 3D imaging has some advantages, guiding interventional procedures with reference to 2D images (and other uses) may still have benefits over at least some of the new 3D imaging and display techniques, including the ability to provide alignment with target tissues using quantitative and qualitative planar positioning guidelines that have been developed over the years.

Despite the advantages of the newly proposed fluid-driven robotic catheter and imaging systems, as with all successes, still further improvements and alternatives would be desirable. In general, it would be beneficial to provide further improved medical devices, systems, and methods, as well as to provide alternative devices, systems, and methods for users to input, view, and control the automated movements thereof. For example, the position and morphology of a diseased heart tissue relating to a structural heart therapy may change significantly between the day on which diagnostic and treatment planning images are obtained, and the day and time an interventional cardiologist begins deploying a therapy within the beating heart. These changes may limit the value of treatment planning prior to start of an interventional procedure based on imaging acquired before the treatment day or time. However, excessive engagement of structural heart devices against sensitive tissues of the heart, as might be imposed when attempting to advance a structural heart tool along and from a constrained access pathway (such as a vascular access lumen, a trans-septal perforation, and/or an outer sheath in the form of a guide catheter or introducer sheath) to a target position in an open chamber of the heart could induce arrhythmias and other trauma. Hence, technologies which facilitate precise movements of these tools and/or in situ trajectory planning for at least a portion of the overall tool movement, ideally while the tool is near or in the target treatment site, would be particularly beneficial.

### BRIEF SUMMARY OF THE INVENTION

The present invention generally provides improved devices, systems, and methods for using, training for the use of, planning for the use of, and/or simulating the use of elongate bodies and other tools such as catheters, borescopes, continuum robotic manipulators, rigid endoscopic robotic manipulators, and the like. The technologies described herein can facilitate precise control over both actual and virtual catheter-based therapies by, for example, allowing medical professionals to plan an automated movement of a therapeutic tool supported by the catheter based on a starting location of the actual catheter as inserted into the heart, and ideally prior to advancement of the tool through at least a portion of an open chamber of the heart. Optionally, a virtual version of the tool can be safely moved from that starting location along a meandering path through a number of different locations till the user has identified a desired position and orientation of the tool for the movement. A processor of the system can then generate synchronized actuator drive signals to move the tool in the chamber of the heart from its starting point to the end without following the meandering input path, with the progress of the tool often being along a virtual axis of the elongate support body as calculated to support the tool in the desired position and orientation. Note that the actual position and orientation of the tool may be offset from the virtual desired axis, with the axis being used to set a goal for lateral articulation of one or more independently driven bendable segments. To facilitate control of the distal portion of a catheter as it advances from the constrained path into the open chamber of the heart in cantileaver, robotic processors and controllers described herein will adjust lateral bend commands based on a length of a constrained portion of a laterally bendable segment, and/or on a stiffness of the tissue or support structure constraining the portion.

In a first aspect, the invention provides a method for aligning a tool with a target tissue adjacent a workspace within a patient body. The method comprises advancing an elongate structure axially along an access path within the patient body. A first segment of the elongate structure is configured to laterally bend in response to a first segment drive command. The advancing can be performed so that a first portion of the first segment is laterally unconstrained within the workspace and a second portion of the first segment is laterally constrained along the access path. The first segment drive command can be determined, with a robotic controller, from a desired tool position and a first portion axial length of the first portion. The first segment drive command may be transmitted from the robotic controller so that the unconstrained first portion of the first segment bends laterally and the tool moves toward the desired tool position.

A number of independent optional features may be provided, alone or in combination, to benefit functionality of the devices and methods described herein. For example, the desired tool position may be defined by a desired tool path, and the advancing, determining, and transmitting can be repeated so that the tool advances along the tool path. Optionally, the path can be defined by determining an aligned pose of the elongate structure with the tool aligned with the target tissue, wherein the elongate structure has an axis and the path extends along the axis of the elongate structure in the aligned pose. The aligned pose can be determined by driving a virtual model of the elongate structure with reference to a display showing an image of the target tissue, the driving of the virtual model including modeled lateral bending of the first segment and modeled axial advancement of the elongate structure, and superimposing images of the virtual model of the elongate structure on the image of the target tissue in the display. Optionally, the virtual model may be driven axially by the processor in response to determining that a command from the user is distal of a boundary of the workspace for the virtual model, and by advancing the virtual model axially. A candidate path of the elongate structure along the axis of the elongate structure in the aligned pose can optionally be validated by superimposing a virtual validation model of the elongate structure on the image of the target tissue in the display and moving the virtual validation model along the candidate path, with the user ideally observing this validation model movement. The user can set a different aligned pose if the candidate path looks to present undesirable risks, or follow-thru with movement it risks appear limited. Automated interference detection may be used instead of or with user observation for validation. The advancing of the elongate structure may be measured with an axial sensor, the axial sensor generating axial sensor signals and the controller determining the first segment drive commands in response to the axial signals. The axial advancing may optionally be performed manually, so that manual advancement of a catheter or the like may be sensed, drive signals are determined by a robotic controller from the sensed signals, and the device may be robotically driven to bend in response to the manual insertion to follow a path derived from the axis of the catheter at the desired end position.

Optionally, the access path may include a lumen of an outer sheath (such as a steerable or non-steerable guide catheter, an introducer sheath, or the like) having a lateral bending stiffness. The first segment drive signal can b determined using the lateral bending stiffness of the outer sheath.

A second segment of the elongate structure can be configured to laterally bend in response to a second segment drive command, the second segment being axially offset from the first segment. The second segment may be proximal or distal of the first segment, and an axial series of more than two laterally bendable segments may also be included in the elongate structure. Any or all of the segments may optionally be driven to bend laterally using fluid and an array of articulation balloons, or they may have pullwires and a resiliently deformable backbone or an axial series of vertebrae coupled to each other with rolling joints. A wide range of alternative continuum robot structures may also be employed, with many of the elongate structures having resilient segments that, when articulated in response to the drive signals, are biased toward a bend extending along the segment length. When laterally restrained, a portion or all of the bent segment can be straight (or less bent than if allowed to assume an unconstrained shape). When the first segment is proximal of the second segment, the robotic controller may determine the second segment drive command from the desired tool position and the first portion axial length, and the first segment axial length of the first segment so that the first and second drive commands together so as to move the tool toward the desired tool position. Alternatively, when the first segment is distal of the second segment, the robotic controller may determine the first segment drive command in response to constrained lateral bending of the second segment, such as using a more limited range of motion of the constrained second segment.

In another aspect, the invention provides a method for aligning a tool with a target tissue adjacent a workspace within a patient body. The method comprises driving, with reference to a display, a virtual model of the elongate structure from an actual pose of the elongate structure to a desired pose. The display may show an image of the elongate structure, an image of the target tissue, and a superimposed image of the virtual model. The elongate structure will typically have an axis, and may be advanced axially along a desired path within the patient body, with the movement along the path performed by transmitting drive commands to laterally bend the elongate body. The desired path can be defined by the axis of the elongate body in the desired pose.

In another aspect, the invention provides method for aligning a tool with a target adjacent a workspace. The method comprises driving a continuum robot per a first drive signal in response to a first command to move a distal end of the continuum robot toward the target. The continuum robot has proximal and distal articulatable portions, and the first signal can be determined with a first number of degree of freedom by a robotic controller in response to the proximal portion being laterally constrained. The continuum robot is driven per a second drive signal in response to a second command to move the distal end toward the target, wherein the second signal is determined with a second number of degree of freedom by the robotic controller in response to the proximal portion being laterally unconstrained. The second number is larger than the first number.

In another aspect, the invention provides a system for aligning a tool with a target tissue adjacent a workspace within a patient body. The system comprises an elongate structure having a proximal end and a distal end with an axis therebetween. The elongate structure may be configured to advance axially along an access path within the patient body. A first segment of the elongate structure may have a first segment length and may be configured to bend laterally in response to a first segment drive command. A sensing system may optionally be coupled to the first segment and may be configured to generate an axial signal in response to a constrained axial portion length of the first segment. A robotic controller may be coupled to the first segment, and the robotic controller can be configured to generate the first segment drive command from a desired tool position and the first segment length (as provided by the sensing system, as entered by the user, or otherwise) so that the unconstrained first portion of the first segment bends laterally and the tool moves toward the desired tool position.

Optional independent features may be included to enhance the system capabilities for specific applications. For example, the controller will often be configured to calculate a plurality of effective lateral bending spring constants of different portions of the catheter body. Such spring constants may vary with the unconstrained length. The catheter body may be constrained by a tubular guide catheter receiving the catheter body therein, with the catheter being axially slidable within the tubular guide. Such a guide may have a lateral bending spring constant that is higher than (and thus be stiffer than) the catheter body, and the controller may determine bending of the constrained portion of the catheter body using the guide spring constant, or the guide may be sufficiently as to be effectively rigid so that the constrained portion lateral deflection can be disregarded (typically with deflections of less than 5 diameters, preferably with deflections of less than 2 or even 1 diameter of the guide). The controller may calculate movement using different degrees of freedom for the system when different portions and/or independently articulatable segments of the catheter body extend beyond the guide, and particularly when a portion of a first, the full length of the first, the first and a portion of a second, and/or the full length of the first and second segments are distal of the guide. Such movement calculations may be used for calculations associated with the forward kinematics of the system, control of the system when a commanded position is near, at, or beyond a boundary of a system workspace (preferably using gradient-based control as described herein), for inverse kinematic calculations, and the like.

In another aspect, the invention provides a system for aligning a tool with a target tissue adjacent a workspace within a patient body. The system comprises an elongate structure having a proximal end and a distal end with an axis therebetween. The elongate structure can be configured to advance axially into a patient body. A simulation module can be configured to drive, with reference to a display, a virtual model of the elongate structure from an actual pose of the elongate structure to a desired pose. The display, in use, may show an image of the elongate structure and an image of the target tissue. The simulation system can be configured to superimpose an image of the virtual model on the display. A robotic controller can be coupled with the elongate structure and configured to help advance the elongate structure axially along a desired path within the patient body by transmitting drive commands to laterally bend the elongate body. The desired path may be defined by the axis of the virtual model of the elongate body in the desired pose.

In another aspect, the invention provides a system for aligning a tool with a target adjacent a workspace. The system comprises a continuum robot having a proximal end and a distal end with an axis therebetween. The continuum robot has axially offset proximal and distal articulatable portions and is configured to advance axially from a lateral constraint into the workspace. A robotic controller can be coupled with the continuum robot, the controller having a first mode when the proximal portion is laterally constrained for transmitting, to the continuum robot, a first drive signal in response to a first command, The continuum robot can be configured so as to, in response, move the distal end toward the target. The first signal can have a first number of degrees of freedom. The robotic controller can also have a second mode when the proximal portion is unconstrained for driving the continuum robot per a second drive signal in response to a second command to move the distal end toward the target. The second signal can have a second number of degrees of freedom, the second number being larger than the first number.

### BRIEF DESCRIPTION OF THE DRAWINGS

FIG. 1 illustrates an interventional cardiologist performing a structural heart procedure with a robotic catheter system having a fluidic catheter driver slidably supported by a stand.

FIG. 2 is a simplified schematic illustration of components of a helical balloon assembly, showing how an extruded multi-lumen shaft can provide fluid to laterally aligned subsets of balloons within an articulation balloon array of a catheter.

FIGS. 3A-3C schematically illustrate helical balloon assemblies supported by flat springs and embedded in an elastomeric polymer matrix, and also show how selective inflation of subsets of the balloons can elongate and laterally articulate the assemblies.

FIG. 4 is a perspective view of a robotic catheter system in which a catheter is removably mounted on a driver assembly, and in which the driver assembly includes a driver encased in a sterile housing and supported by a stand.

FIG. 5 schematically illustrates a robotic catheter system and transmission of signals between the components thereof so that input from a user induces a desired articulation.

FIG. 6 is a high-level flow chart of an exemplary control system for the fluid-driven robotic catheter control systems described herein.

FIG. 7 is a flow chart showing an exemplary method and structure for use by the control systems described herein to solve for the inverse kinematics of the fluid-driven catheter structures.

FIG. 8 illustrates a relationship between reference frames at the base of the articulated segments and at the tip of the catheter, for use in the control systems described herein.

FIG. 9 illustrates Euler angles for determination of transformations between reference frames used in the control systems described herein.

FIGS. 10A-10C graphically illustrate coordinate frames, angle and length symbols representing variable values used by the control systems described herein.

FIG. 11 is a flow chart showing inverse kinematics for a segment as can be used to solve for lumen pressures in a control system of the fluid-driven catheter systems described herein.

FIG. 11A graphically illustrates segment and other component lengths of a catheter system, and shows how an outer sheath or other structure may laterally constrain a portion of a segment by increasing a spring constant of the portion of a segment disposed therein.

FIG. 12 graphically illustrates angles and values used in the user interface of the control systems described herein.

FIGS. 13A and 13B graphically illustrate angles and values used for communication of signals between the user interface and the robotic position control of the control systems described herein.

FIG. 14 schematically illustrate exemplary components of an input-output system for use in the robotic control and/or simulation systems described herein, and also show an image of a tool supported by a flexible body within an internal surgical site bordered by adjacent tissue.

FIGS. 15A and 15B schematically illustrate exemplary components of robotic control and/or simulation systems and show communications between those components.

FIGS. 16A-16D are screen prints of display images showing an in situ movement plan (or simulation thereof) and associated movement of a catheter-supported tool along a trajectory determined by disregarding one or more candidate intermediate input poses and a trajectory of a virtual catheter indicator.

FIGS. 17A-17D are screen prints of display images showing rotational and translational movements and associated indicia for use with a planar input device during movement of an actual or simulated robotic catheter.

FIGS. 18A-18C illustrate exemplary graphical indicia superimposed on an image of an actual or simulated robotic catheter to facilitate precise and predictable rotation, translation, and alignment with target tissues.

FIG. 19 is a functional block diagram of an exemplary fluid-driven structural heart therapy system having an augmented reality hybrid 2D/3D display for reference by a system user to position a therapeutic or diagnostic tool in an open chamber of a patient's beating heart.

FIGS. 20A and 20B are screen shots of an augmented reality display for use in the system of FIG. 19, showing a captured 2D image representing an actual tip of an articulated delivery system and adjacent tissue in FIG. 20A, and showing a 2D image of a virtual model of the articulated delivery system superimposed on the captured 2D tissue/tip image.

FIG. 21 is a screenshot showing a hybrid 2D/3D display for use in the system of FIG. 19, with the display presenting an image including a 3D virtual model of the articulated delivery system, and also presenting first and second 2D image planes, each having a 2D image of the virtual model projected thereon with the orientations of the 2D images relative to the model corresponding to the orientations of the planes on which they are projected, wherein the 2D image planes represent fluoroscopic image planes.

FIG. 22 is a screenshot showing another hybrid 2D/3D display presenting a 3D virtual image, a 2D fluoroscopic image plane, and first and second 2D ultrasound image slice planes, wherein the ultrasound planes are offset from the model.

FIG. 23 is a screenshot showing another hybrid 2D/3D display presenting a 3D virtual image of an articulated delivery system and an ultrasound transducer, a 2D fluoroscopic image plane having the articulated delivery system and transducer projected thereon, and first and second 2D ultrasound image slice planes of the transducer, and a 3D ultrasound image volume of the transducer so as to illustrate how image data of the fluoroscopic and ultrasound system can be registered and tracked.

FIG. 24 is a screenshot showing first and second 2D ultrasound image slice planes of a transducer, along with a 3D virtual image of an articulated delivery system and 2D virtual image sliced projected from the model to the 2D ultrasound image planes.

FIG. 25 is a screenshot showing yet another hybrid 2D/3D display showing a 3D virtual model of the articulated delivery system, offset of first and second 2D ultrasound image planes along their normals, projection of a phantom articulated delivery system onto the ultrasound image planes, and a 3D trajectory between the virtual 3D model and the 3D phantom.

FIGS. 26A-26C are screenshots showing a hybrid 2D/3D display showing 3D virtual models of articulated delivery systems and 2D images of the virtual models projected on 2D images, with both the 3D and 2D images including widgets adjacent the tip of the models, the widgets correlating to a constraint on the movement of the articulated delivery system.

FIGS. 26D-26G schematically illustrate geometric terms which may optionally be used to constrain motion of articulated devices as detailed in the associated text.

FIGS. 27A-27C are screenshots showing pages of a 6 degree of freedom input device, showing app pages and buttons for controlling the articulation system in different modes using a variety of alternative constraints.

FIGS. 28A-28C illustrate manual positioning of a 6 degree of freedom input device and corresponding movement of an image of a 3D virtual catheter within a 3D virtual workspace as shown on a 2D display, and also show spring-back of the view to a starting position and orientation when a view drive input button is released.

FIGS. 29A-29D illustrate optional image elements to be included in an exemplary hybrid 2D/3D display image.

FIGS. 30A-30C illustrate manual manipulation of the catheter body from outside the patient while driving the tip so as to inhibit resulting changes in the tip position.

FIG. 31 schematically illustrates a calculated trajectory that avoids a workspace boundary, along with a virtual trajectory verification catheter that moves along the trajectory to facilitate visual verification of safety prior to implementing a move of the actual catheter.

FIG. 32A illustrates a perspective view of a multi-segment articulated catheter (the steerable sleeve) supporting an instrument or tool (specifically a clip), with an instrument deployment catheter extending through the segments and a clip being supported by the deployment catheter distal of the segments; the clip, deployment catheter, and segments being introduced into a chamber of a heart or other workspace within a patient through a guide catheter.

FIGS. 32B, 32C, and 32D illustrates schematic views of an instrument, of a multi-segment articulated catheter and a guide catheter, and also identify locations along the catheter system and associated spring constants and catheter portion lengths.

FIG. 32E is a chart that defines in symbols and equations the relative positions and spring constants of sections of the catheter system for driving with different degrees of freedom in response to different portions of the catheter being laterally unconstrained and/or constrained.

### DETAILED DESCRIPTION OF THE INVENTION

The improved devices, systems, and methods for controlling, image guidance of, inputting commands into, and simulating movement of powered and robotic devices will find a wide variety of uses. The elongate tool-supporting structures described herein will often be flexible, typically comprising catheters suitable for insertion in a patient body. Exemplary systems will be configured for insertion into the vascular system, the systems typically including a cardiac catheter and supporting a structural heart tool for repairing or replacing a valve of the heart, occluding an ostium or passage, or the like. Other cardiac catheter systems will be configured for diagnosis and/or treatment of congenital defects of the heart, or may comprise electrophysiology catheters configured for diagnosing or inhibiting arrhythmias (optionally by ablating a pattern of tissue bordering or near a heart chamber).

Alternative applications may include use in steerable supports of image acquisition devices such as for trans-esophageal echocardiography (TEE), intra-coronary echocardiography (ICE), and other ultrasound techniques, endoscopy, and the like. The structures described herein will often find applications for diagnosing or treating the disease states of or adjacent to the cardiovascular system, the alimentary tract, the airways, the urogenital system, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with one or more (preferably multiple) degrees of freedom without having to resort to complex rigid linkages.

Embodiments provided herein may use balloon-like structures to effect articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medial structures described herein will often have an articulated distal portion, and an unarticulated proximal portion, which may significantly simplify initial advancement of the structure into a patient using standard catheterization techniques.

The robotic systems described herein will often include an input device, a driver, and an articulated catheter or other robotic manipulator supporting a diagnostic or therapeutic tool. The user will typically input commands into the input device, which will generate and transmit corresponding input command signals. The driver will generally provide both power for and articulation movement control over the tool. Hence, somewhat analogous to a motor driver, the driver structures described herein will receive the input command signals from the input device and will output drive signals to the tool-supporting articulated structure so as to effect robotic movement of an articulated feature of the tool (such as movement of one or more laterally deflectable segments of a catheter in multiple degrees of freedom). The drive signals may comprise fluidic commands, such as pressurized pneumatic or hydraulic flows transmitted from the driver to the tool-supporting catheter along a plurality of fluid channels. Optionally, the drive signals may comprise electromagnetic, optical, or other signals, preferably (although not necessarily) in combination with fluidic drive signals. Unlike many robotic systems, the robotic tool supporting structure will often (though not always) have a passively flexible portion between the articulated feature (typically disposed along a distal portion of a catheter or other tool manipulator) and the driver (typically coupled to a proximal end of the catheter or tool manipulator). The system will be driven while sufficient environmental forces are imposed against the tool or catheter to impose one or more bend along this passive proximal portion, the system often being configured for use with the bend(s) resiliently deflecting an axis of the catheter or other tool manipulator by 10 degrees or more, more than 20 degrees, or even more than 45 degrees.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transvers to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the curved outer surface of the cylinder between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

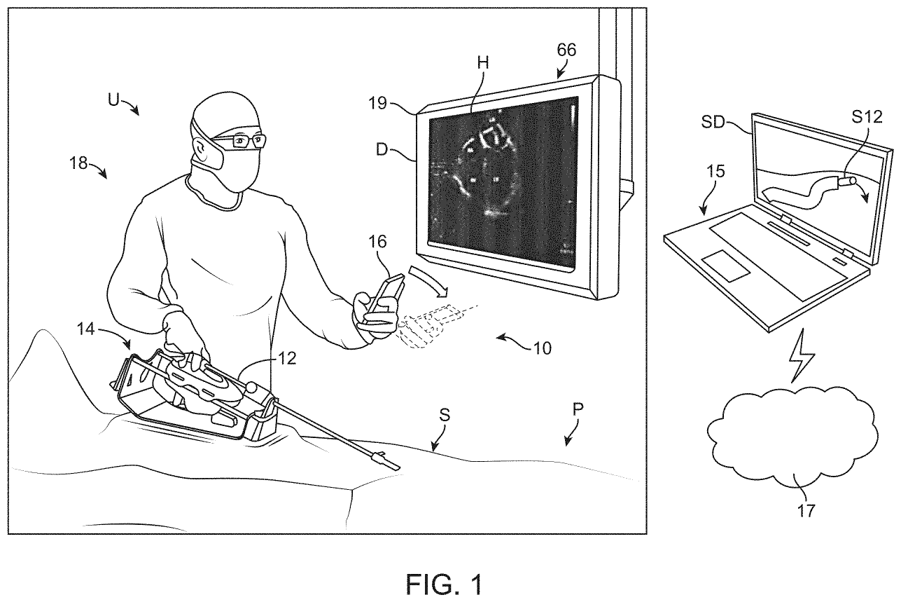

Referring first to FIG. 1, a system user U, such as an interventional cardiologist, uses a robotic catheter system 10 to perform a procedure in a heart H of a patient P. System 10 generally includes an articulated catheter 12, a driver assembly 14, and an input device 16. User U controls the position and orientation of a therapeutic or diagnostic tool mounted on a distal end of catheter 12 by entering movement commands into input 16, and optionally by sliding the catheter relative to a stand of the driver assembly, while viewing a distal end of the catheter and the surrounding tissue in a display D. As will be described below, user U may alternatively manually rotate the catheter body about its axis in some embodiments.

During use, catheter 12 extends distally from driver system 14 through a vascular access site S, optionally (though not necessarily) using an introducer sheath. A sterile field 18 encompasses access site S, catheter 12, and some or all of an outer surface of driver assembly 14. Driver assembly 14 will generally include components that power automated movement of the distal end of catheter 12 within patient P, with at least a portion of the power often being transmitted along the catheter body as a hydraulic or pneumatic fluid flow. To facilitate movement of a catheter-mounted therapeutic tool per the commands of user U, system 10 will typically include data processing circuitry, often including a processor within the driver assembly. Regarding that processor and the other data processing components of system 10, a wide variety of data processing architectures may be employed. The processor, associated pressure and/or position sensors of the driver assembly, and data input device 16, optionally together with any additional general purpose or proprietary computing device (such as a desktop PC, notebook PC, tablet, server, remote computing or interface device, or the like) will generally include a combination of data processing hardware and software, with the hardware including an input, an output (such as a sound generator, indicator lights, printer, and/or an image display), and one or more processor board(s). These components are included in a processor system capable of performing the transformations, kinematic analysis, and matrix processing functionality associated with generating the valve commands, along with the appropriate connectors, conductors, wireless telemetry, and the like. The processing capabilities may be centralized in a single processor board, or may be distributed among various components so that smaller volumes of higher-level data can be transmitted. The processor(s) will often include one or more memory or other form of volatile or non-volatile storage media, and the functionality used to perform the methods described herein will often include software or firmware embodied therein. The software will typically comprise machine-readable programming code or instructions embodied in non-volatile media and may be arranged in a wide variety of alternative code architectures, varying from a single monolithic code running on a single processor to a large number of specialized subroutines, classes, or objects being run in parallel on a number of separate processor sub-units.

Referring still to FIG. 1, along with display D, a simulation display SD may present an image of an articulated portion of a simulated or virtual catheter S12 with a receptacle for supporting a simulated therapeutic or diagnostic tool. The simulated image shown on the simulation display SD may optionally include a tissue image based on pre-treatment imaging, intra-treatment imaging, and/or a simplified virtual tissue model, or the virtual catheter may be displayed without tissue. Simulation display SD may have or be included in an associated computer 15, and the computer will preferably be couplable with a network and/or a cloud 17 so as to facilitate updating of the system, uploading of treatment and/or simulation data for use in data analytics, and the like. Computer 15 may have a wireless, wired, or optical connection with input device 16, a processor of driver assembly 14, display D, and/or cloud 17, with suitable wireless connections comprising a Bluetooth™ connection, a WiFi connection, or the like. Preferably, an orientation and other characteristics of simulated catheter S12 may be controlled by the user U via input device 16 or another input device of computer 15, and/or by software of the computer so as to present the simulated catheter to the user with an orientation corresponding to the orientation of the actual catheter as sensed by a remote imaging system (typically a fluoroscopic imaging system, an ultra-sound imaging system, a magnetic resonance imaging system (MM), or the like) incorporating display D and an image capture device 19. Optionally, computer 15 may superimpose an image of simulated catheter S12 on the tissue image shown by display D (instead of or in addition to displaying the simulated catheter on simulation display SD), preferably with the image of the simulated catheter being registered with the image of the tissue and/or with an image of the actual catheter structure in the surgical site. Still other alternatives may be provided, including presenting a simulation window showing simulated catheter SD on display D, including the simulation data processing capabilities of computer 15 in a processor of driver assembly 14 and/or input device 16 (with the input device optionally taking the form of a tablet that can be supported by or near driver assembly 14, incorporating the input device, computer, and one or both of displays D, SD into a workstation near the patient, shielded from the imaging system, and/or remote from the patient, or the like.

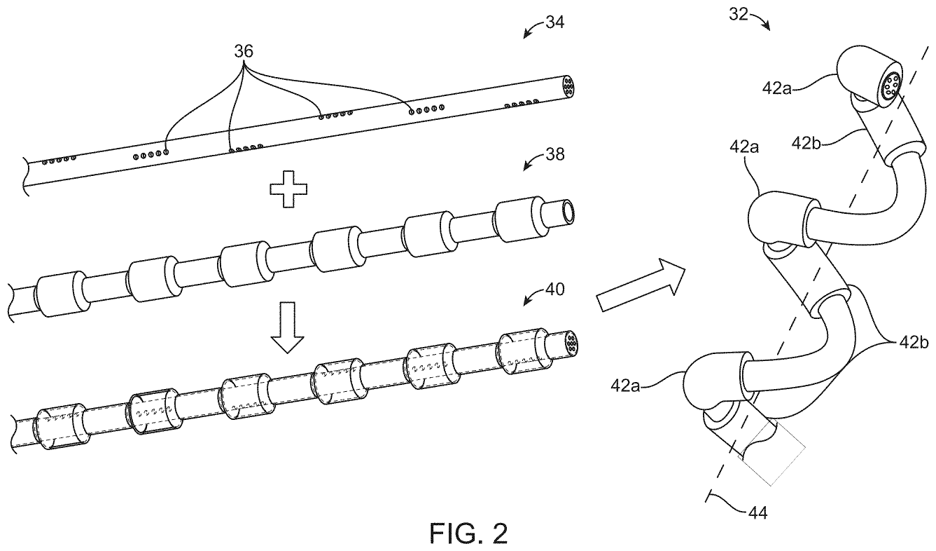

Referring now to FIG. 2, the components of, and fabrication method for production of, an exemplary balloon array assembly (sometimes referred to herein as a balloon string 32) can be understood. A multi-lumen shaft 34 will typically have between 3 and 18 lumens. The shaft can be formed by extrusion with a polymer such as a nylon, a polyurethane, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like. A series of ports 36 are formed between the outer surface of shaft 36 and the lumens, and a continuous balloon tube 38 is slid over the shaft and ports, with the ports being disposed in large profile regions of the tube and the tube being sealed over the shaft along the small profile regions of the tube between ports to form a series of balloons. The balloon tube may be formed using a compliant, non-compliant, or semi-compliant balloon material such as a latex, a silicone, a nylon elastomer, a polyurethane, a nylon, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like, with the large-profile regions preferably being blown sequentially or simultaneously to provide desired hoop strength. The ports can be formed by laser drilling or mechanical skiving of the multi-lumen shaft with a mandrel in the lumens. Each lumen of the shaft may be associated with between 3 and 50 balloons, typically from about 5 to about 30 balloons. The shaft balloon assembly 40 can be coiled to a helical balloon array of balloon string 32, with one subset of balloons 42a being aligned along one side of the helical axis 44, another subset of balloons 44b (typically offset from the first set by 120 degrees) aligned along another side, and a third set (shown schematically as deflated) along a third side. Alternative embodiments may have four subsets of balloons arranged in quadrature about axis 44, with 90 degrees between adjacent sets of balloons.

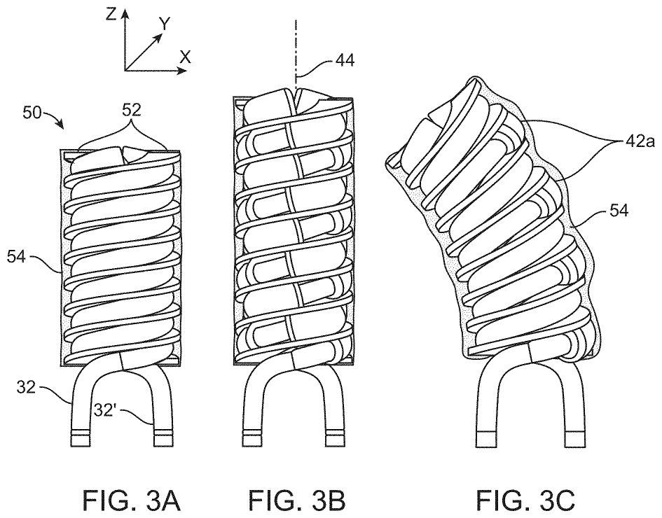

Referring now to FIGS. 3A, 3B, and 3C, an articulated segment assembly 50 has a plurality of helical balloon strings 32, 32′ arranged in a double helix configuration. A pair of flat springs 52 are interleaved between the balloon strings and can help axially compress the assembly and urge deflation of the balloons. As can be understood by a comparison of FIGS. 3A and 3B, inflation of subsets of the balloons surrounding the axis of segment 50 can induce axial elongation of the segment. As can be understood with reference to FIGS. 3A and 3C, selective inflation of a balloon subset 42a offset from the segment axis 44 along a common lateral bending orientation X induces lateral bending of the axis 44 away from the inflated balloons. Variable inflation of three or four subsets of balloons (via three or four channels of a single multi-lumen shaft, for example) can provide control over the articulation of segment 50 in three degrees of freedom, i.e., lateral bending in the +/−X orientation and the +/−Y orientation, and elongation in the +Z orientation. As noted above, each multilumen shaft of the balloon strings 32, 32′ may have more than three channels (with the exemplary shafts having 6 or 7 lumens), so that the total balloon array may include a series of independently articulatable segments (each having 3 or 4 dedicated lumens of one of the multi-lumen shafts, for example). Optionally, from 2 to 4 modular, axially sequential segments may each have an associated tri-lumen shaft, with the tri-lumen shaft extending axially in a loose helical coil through the lumen of any proximal segments to accommodate bending and elongation. The segments may each include a single helical balloon string/multilumen shaft assembly (rather than having a dual-helix configuration). Multi-lumen shafts for driving of distal segments may alternatively wind proximally around an outer surface of a proximal segment, or may be wound parallel and next to the multi-lumen shaft/balloon tube assemblies of the balloon array of the proximal segment(s).

Referring still to FIGS. 3A, 3B, and 3C, articulated segment 50 optionally includes a polymer matrix 54, with some or all of the outer surface of balloon strings 32, 32′ and flat springs 52 that are included in the segment being covered by the matrix. Matrix 54 may comprise, for example, a relatively soft elastomer to accommodate inflation of the balloons and associated articulation of the segment, with the matrix optionally helping to urge the balloons toward an at least nominally deflated state, and to urge the segment toward a straight, minimal length configuration. Alternatively (or in addition to a relatively soft matrix), a thin layer of relatively high-strength elastomer can be applied to the assembly (prior to, after, or instead of the soft matrix), optionally while the balloons are in an at least partially inflated state. Advantageously, matrix 54 can help maintain overall alignment of the balloon array and springs within the segment despite segment articulation and bending of the segment by environmental forces. Regardless of whether or not a matrix is included, an inner sheath may extend along the inner surface of the helical assembly, and an outer sheath may extend along an outer surface of the assembly, with the inner and/or outer sheaths optionally comprising a polymer reinforced with wire or a high-strength fiber in a coiled, braid, or other circumferential configuration to provide hoop strength while accommodating lateral bending (and preferably axial elongation as well). The inner and outer sheaths may be sealed together distal of the balloon assembly, forming an annular chamber with the balloon array disposed therein. A passage may extend proximally from the annular space around the balloons to the proximal end of the catheter to safely vent any escaping inflation media, or a vacuum may be drawn in the annular space and monitored electronically with a pressure sensor to inhibit inflation flow if the vacuum deteriorates.

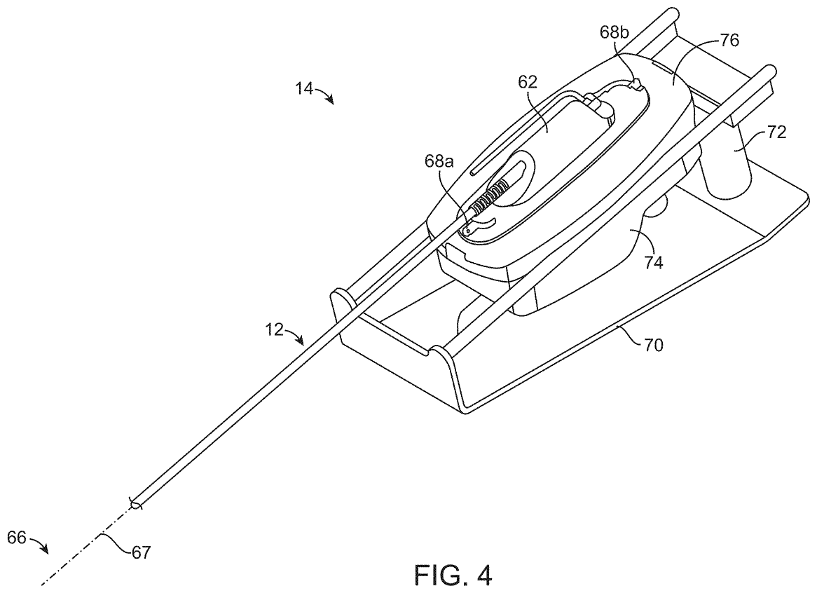

Referring now to FIG. 4, a proximal housing 62 of catheter 12 and the primary components of driver assembly 14 can be seen in more detail. Catheter 12 generally includes a catheter body 64 that extends from proximal housing 62 to an articulated distal portion 66 (see FIG. 1) along an axis 67, with the articulated distal portion preferably comprising a balloon array and the associated structures described above. Proximal housing 62 also contains first and second rotating latch receptacles 68a, 68b which allow a quick-disconnect removal and replacement of the catheter. The components of driver assembly 14 visible in FIG. 4 include a sterile housing 70 and a stand 72, with the stand supporting the sterile housing so that the sterile housing (and components of the driver assembly therein, including the driver) and catheter 12 can move axially along axis 67. Sterile housing 70 generally includes a lower housing 74 and a sterile junction having a sterile barrier 76. Sterile junction 76 releasably latches to lower housing 74 and includes a sterile barrier body that extends between catheter 12 and the driver contained within the sterile housing. Along with components that allow articulation fluid flow to pass through the sterile fluidic junction, the sterile barrier may also include one or more electrical connectors or contacts to facilitate data and/or electrical power transmission between the catheter and driver, such as for articulation feedback sensing, manual articulations sensing, or the like. The sterile housing 70 will often comprise a polymer such as an ABS plastic, a polycarbonate, acetal, polystyrene, polypropylene, or the like, and may be injection molded, blow molded, thermoformed, 3-D printed, or formed using still other techniques. Polymer sterile housings may be disposable after use on a single patient, may be sterilizable for use with a limited number of patients, or may be sterilizable indefinitely; alternative sterile housings may comprise metal for long-term repeated sterile processing. Stand 72 will often comprise a metal, such as a stainless steel, aluminum, or the like for repeated sterilizing and use.

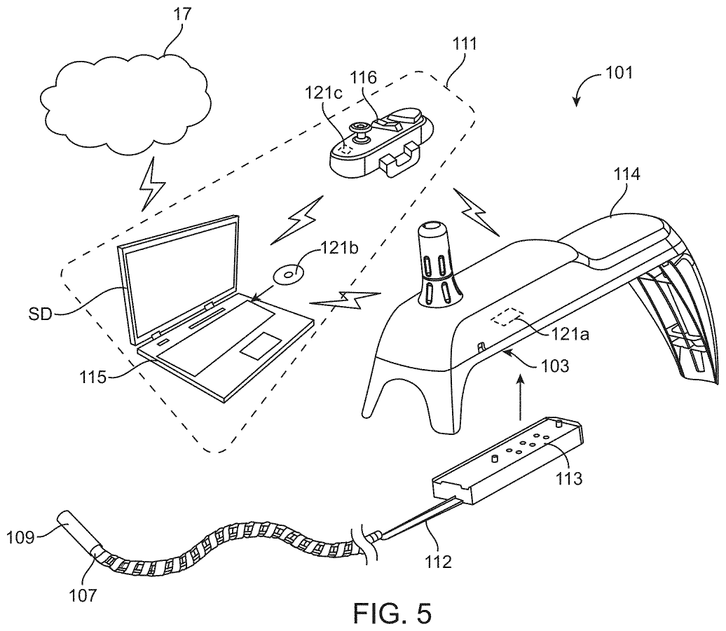

Referring now to FIG. 5, components of a simulation system 101 that can be used for simulation, training, pre-treatment planning, and or treatment of a patent are schematically illustrated. Some or all of the components of system 101 may be used in addition to or instead of the clinical components of the system shown in FIG. 1. System 101 may optionally include an alternative catheter 112 and an alternative driver assembly 114, with the alternative catheter comprising a real and/or virtual catheter and the driver assembly comprising a real and/or virtual driver 114.

Alternative catheter 112 can be replaceably coupled with alternative driver assembly 114. When simulation system 101 is used for driving an actual catheter, the coupling may be performed using a quick-release engagement between an interface 113 on a proximal housing of the catheter and a catheter receptacle 103 of the driver assembly. An elongate body 105 of catheter 112 has a proximal/distal axis as described above and a distal receptacle 107 that is configured to support a therapeutic or diagnostic tool 109 such as a structural heart tool for repairing or replacing a valve of a heart. The tool receptacle may comprise an axial lumen for receiving the tool within or through the catheter body, a surface of the body to which the tool is permanently affixed, or the like. Alternative drive assembly 114 may be wireless coupled to a simulation computer 115 and/or a simulation input device 116, or cables may be used for transmission of data.

When alternative catheter 112 and alternative drive system 114 comprise virtual structures, they may be embodied as modules of software, firmware, and/or hardware. The modules may optionally be configured for performing articulation calculations modeling performance of some or all of the actual clinical components as described below, and/or may be embodied as a series of look-up tables to allow computer 115 to generate a display effectively representing the performance. The modules will optionally be embodied at least in-part in a non-volatile memory of a simulation-supporting alternative drive assembly 121a, but some or all of the simulation modules will preferably be embodied as software in non-volatile memories 121b, 121c of simulation computer 115 and/or simulation input device 116, respectively. Coupling of alternative virtual catheters and tools can be permed using menu options or the like. In some embodiments, selection of a virtual catheter may be facilitated by a signal generated in response to mounting of an actual catheter to an actual driver.

Simulation computer 115 preferably comprises an off-the-shelf notebook or desktop computer that can be coupled to cloud 17, optionally via an intranet, the internet, an ethernet, or the like, typically using a wireless router or a cable coupling the simulation computer to a server. Cloud 17 will preferably provide data communication between simulation computer 115 and a remote server, with the remote server also being in communication with a processor of other simulation computers 115 and/or one or more clinical drive assemblies 14. Simulation computer 115 may also comprise code with a virtual 3D workspace, the workspace optionally being generated using a proprietary or commercially available 3D development engine that can also be used for developing games and the like, such as Unity™ as commercialized by Unity Technologies. Suitable off-the-shelf computers may include any of a variety of operating systems (such as Windows from Microsoft, OS from Apple, Linex, or the like), along with a variety of additional proprietary and commercially available apps and programs.

Simulation input device 116 may comprise an off-the-shelf input device having a sensor system for measuring input commands in at least two degrees of freedom, preferably in 3 or more degrees of freedom, and in some cases 5, 6, or more degrees of freedom. Suitable off-the-shelf input devices include a mouse (optionally with a scroll wheel or the like to facilitate input in a 3^{rd} degree of freedom), a tablet or phone having an X-Y touch screen (optionally with AR capabilities such as being compliant with ARCor from Google, ARKit from Apple, or the like to facilitate input of translation and/or rotation, along with multi-finger gestures such as pinching, rotation, and the like), a gamepad, a 3D mouse, a 3D stylus, or the like. Proprietary code may be loaded on the simulation input device (particularly when a phone, tablet, or other device having a touchscreen is used), with such input device code presenting menu options for inputting additional commands and changing modes of operation of the simulation or clinical system. A simulation input/output system 111 may be defined by the simulation input device 116 and the simulation display SD.

System Motion Equations

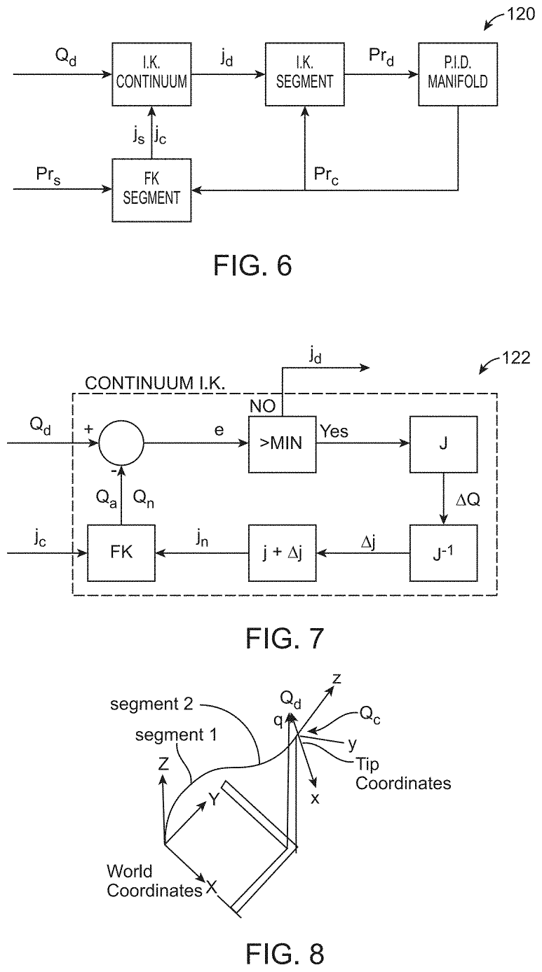

Referring now to FIG. 6, a system control flow chart 120 is shown that can be used by a processor of the robotic system to drive movement of an actual or virtual catheter. The following terms are used in flow chart 120 and/or in the analysis that follows. The following notation may be used in at least some of the equations herein:

### Input

Q_{d} Tip position desired

### Measured

Pr₀ Pressure for balloon arrays

### Calculated

Pr_{d} Pressure for balloon arrays as computed for joint, j_{d}
j Joint space variables
j_{d} Joint space computed for desired tip position, Q_{d}
jₙ Joint space newly computed
+ Add increment to each variable one at a time
Q Tip position in world space
Q+ Tip positions by added increments to each variable one at a time to form Jacobian
Qₙ Tip position newly calculated

### e Error

Seg FK Segment Forward Kinematics; pressures to joint space
Seg IK Segment Inverse Kinematics; joint space to pressures
FK System Forward Kinematics; joint space to world space (distal segments tip position)
IK System Inverse Kinematics; world space (distal segment tip position) to joint space
J Jacobian (numerically derived)

### J⁻¹ Inverse Jacobian

Referring now to FIG. 7, an inverse kinematics flow chart 122 is useful to understand how the processor can solve for joint angles and displacements. The calculations below reference the variables outlined in Table I. The input variables (α0, α1, α2, α3, α4, β1, β2, β3, β4, S0, S1, S2, S3, and S4) are used as input in these calculations. The calculations are arranged for articulated catheters having up to 4 articulated independently articulatable segments (segment Nos. 1-4), and a base segment (No. 0) can be used to accommodate a pose of the catheter body proximally of the most proximal articulated segment (the parameters for the base segment or segment No. 0 being treated as defined parameters in the calculations).

TABLE 1
Variable Matrix

| Segment no. | n | i | 0 | 1 | 2 | 3 | 4 | |
|---|---|---|---|---|---|---|---|---|
| Angles | Alpha | αᵢ | α₀ | α₁ | α₂ | α₃ | α₄ | Input |
| | Beta | βᵢ | | β₁ | β₂ | β₃ | β₄ | |
| Arc Length | Arc | Sᵢ | S₀ | S₁ | S₂ | S₃ | S₄ | |
| Arc Radii | Radius | rᵢ | | r₁ | r₂ | r₃ | r₄ | Solve |
| Arc | Pᵢ | xᵢ | 0 | x₁ | x₂ | x₃ | x₄ | For |
| End | | yᵢ | 0 | y₁ | y₂ | Y₃ | y₄ | |
| Points | | zᵢ | z₀ | z₁ | z₂ | z₃ | _{z4} | |
| Arc | Vᵢ | aᵢ | 0 | a₁ | a₂ | a₃ | a₄ | |
| Unit | | bᵢ | 0 | b₁ | b₂ | b₃ | b₄ | |
| Vectors | | cᵢ | 1 | c₁ | c₂ | c₃ | c₄ | |

Startup Position:

Segments start by expanding the balloon array inflations to predetermined levels. The Segment is driven to predetermined and straight (or nearly straight) condition defined by an initial joint space vector jₛ, which accounts for all the Segments' initial states.

To move to a desired location, balloon array conditions are changed to locate segment angles and displacement. The first step is to determine the current and desired position vectors for the robot tip in the robot's base coordinate system, sometime referred to herein as world space. Note that world space may move relative to the patient and/or the operating room with manual repositioning of the catheter or the like, so that this world space may differ from a fixed room space or global world space.

User Input Commands/Tip Vector q:

Referring now to FIG. 8, The robot is driven by a user input vector q measured from a coordinate system attached to the distal end of the robot, or tip. This q vector is transformed into a desired world space vector Qa, where the current tip location in world space vector is Q_{c}.

Q_{c}=(X_{c},Y_{c},Z_{c}, [symbol] _{c}, [symbol] _{c})

The user input q represents a velocity (or small displacements) command. The tip coordinate system resides at the current tip position, and therefore the current q, or q_{c} is always at the origin:

q_{c}=(0,0, 0, 0, 0)

Since q_{c} is zero the desired displacement, qa, is equivalent to the change in q or dq as shown here:

dq=q_{d}—q_{c}=q_{d}

To simplify, dq is replaced simply with q to represent the desired change in position,

q=q_{d}=(x_{T},y_{T},Z_{T},α_{T},β_{T}),

where x_{T}, y_{T}, z_{T}, α_{T} and β_{T} describes a change vector in tip space coordinates. q is then used by the current Transformation Matrix, T_{0Tc}, to acquire the desired world coordinates, Q_{d}.

Q_{d}=T_{0Tc}(q_{d})=(X_{d},Y_{d},Z_{d}, [symbol] _{d}, [symbol] _{d})

Where the Tip's current world coordinates are defined by Q_{c}.

Q_{c}=T_{0Tc}(q_{c})=(X_{c},Y_{c},Z_{c}, [symbol] _{c}, [symbol] _{c})

Use Joint Space vector, J, contains the Segment angles and displacement information which is used to solve for the Transformation and Rotation matrix, T_{0T} and R_{0T} respectively. The Transformation and Rotation Matrix will be discussed below.

Current Catheter State or Pose

### Q_{c}:

The current world coordinate vector Q_{c} is defined by the tip q vector with no displacement which is q_{c}, and can be resolved as follows:

Q_{c}=T_{0Tc}(q_{c})=(X_{c},Y_{c},Z_{c}, [symbol] _{c}, [symbol] _{c})

The coordinates may be found by the following math,

(X_{c},Y_{c},Z_{c},1)^{T}=T_{0Tc}·(0,0,0,1)^{T}

a_{T}=cos(0)*sin(0)=0

b_{T}=sin(0)*sin(0)=0

c_{T}=cos(0)=1

(A_{c},B_{c},C_{c},0)^{T}=T_{0Tc}·(0,0,1,0)^{T}

[symbol]

_{c}=atan2(A_{c},B_{c})

[symbol]

_{c}=atan2(C_{c},H_{c})

H_{c}=(A_{c}²B_{c}²)^{1/2}

The range of Beta ( [symbol] ) may be limited if the rotation matrix is not used. This is due to use of the hypotenuse (H) quantity which removes the negative sign from one side of the atan2 formula as follows:

H=(A²+B²)^{1/2}

[symbol]

=atan2(C,H),0< [symbol] <180

### Q_{d}:

The desired world coordinate vector Q_{d} is defined by the tip q vector with desired displacement which is q_{d}, and can be resolved as follows:

Q_{d}=T_{0Tc}(q_{d})=(X_{d},Y_{d},Z_{d}, [symbol] _{d}, [symbol] _{d})

The coordinates may be found by the following math,

(X_{d},Y_{d},Z_{d},1)^{T}=T_{0T}·(x_{T},y_{T},z_{T},1)^{T}

a_{T}=cos(α_{T})*sin(β_{T}); b_{T}=sin(α_{T})*sin(β_{T}); C_{T}=COS(β_{T})

(A_{d},B_{d},C_{d},0)^{T}=T_{0T}·(a_{T},b_{T},c_{T},0)^{T}

[symbol]

=atan2(A_{d},B_{d})

[symbol]

_{d}=atan2(C_{d},H_{d})

H_{d}=(A_{d}²+B_{d}²)^{1/2}

The following alternative formula solves Beta (B) in all four quadrants for a full 360 degrees (as opposed to only two quadrans and 180 degrees) and for Gamma (Γ), the sixth and final coordinate to define the position in 3D space.

### Q_{c}:

Use J_{c} (current Joint Space variables) is used to solve for the current T_{0Tc} and R_{0Tc}

(X_{c},Y_{c},Z_{c},1)^{T}=T_{0Tc}·(0,0,0,1)^{T}

(a_{Tx},b_{Tx},c_{Tx})^{T}=R_{0Tc}×(1,0,0)^{T}

(a_{Ty},b_{Ty},c_{Ty})^{T}=R_{0Tc}×(0,1,0)^{T}

(a_{Tz},b_{Tz},C_{Tz})^{T}=R_{0Tc}×(0,0,1)^{T}

[symbol]

_{c}=atan2(a_{Tz}, b_{Tz})

if |a_{Tz}|<min; than a_{Tz}=(a_{Tz}/|a_{Tz}|)*min

[symbol]

_{c}=atan2(c_{αz},a_{αz})

a_{αz}=cos α*a_{Tz}+sin α*b_{Tz}

C_{αz}=C_{Tz}

if |c_{αz}|<min; than c_{αz}=(c_{αz}/|c_{αz}|)*min

Γ_{c}=atan2(a_{βx},b_{βx})

a_{βx}=cos α*cos β*a_{Tx}+sin α*cos β*b_{Tx}−sin β*c_{Tx}

b_{βx}=−sin α*a_{Tx}+cos α*b_{Tx}

if |a_{βx}|<min; than a_{βx}=(a_{βx}/|a_{βx}|)*min

### Q_{d}:

(X_{d},Y_{d},Z_{d},1)^{T}=T_{0Tc}·(x_{d},y_{d},z_{d},1)^{T}

Use Q_{d} with Inverse Jacobian to solve for Segment angles and displacements, J_{d} (desired Joint Space variables).
Use J_{d} to solve for the new T_{0Td} and R_{0Td}

(X_{d},Y_{d},Z_{d},1)^{T}=T_{0Td}·(0,0,0,1)^{T}

(a_{Tx},b_{Tx},c_{Tx})^{T}=R_{0Td}×(1,0,0)^{T}

(a_{Ty},b_{Ty},c_{Ty})^{T}=R_{0Td}×(0,1,0)^{T}

(a_{Tz},b_{Tz},c_{Tx}=R_{0Td}×(0,0,1)^{T}

[symbol]

_{d}=atan2(a_{Tz},b_{Tz})

if |a_{Tz}|<min; than a_{Tz}=(a_{Tz}/|a_{Tz}|)*min

[symbol]

_{d}=atan2(c_{αz},a_{αz})

a_{αz}=cos α*a_{Tz}+sin α*b_{Tz}

c_{αz}=c_{Tz}

if |c_{αz}|<min; than c_{αz}=(c_{αz}/|c_{αz}|)*min

Γ_{d}=atan2(a_{βx},b_{βx})

a_{βx}=cos α*cos β*a_{Tx}+sin α*cos β*b_{Tx}−sin β*c_{Tx}

b_{βx}=−sin α*a_{Tx}+cos α*b_{Tx}

if |a_{βx}|<min; than a_{βx}=(a_{βx}/|a_{βx}|*min

Basis for Alternative Formulas:

Solve for base coordinate axis unit vectors

(a_{Tx},b_{Tx},c_{Tx})^{T}=R_{0T}×(1,0,0)^{T}

(a_{Ty},b_{Ty},c_{Ty})^{T}=R_{0T}×(0,1,0)^{T}

(a_{Tz},b_{Tz},c_{Tx}=R_{0T}×(0,0,1)T

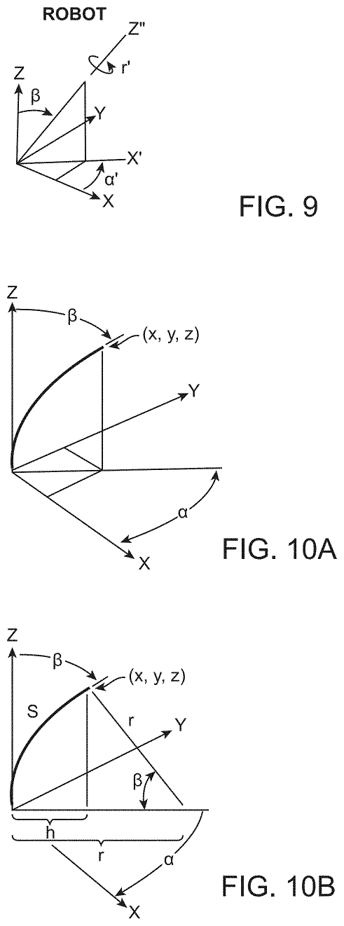

Referring now to FIG. 9, the Robot angles are resolved as follows:

α_{T}=atan2(a_{Tz},b_{Tz})

The rotation matrix for alpha (α) is the following:

$${R}_{α} = ( \begin{matrix}cos α & - s in α & 0 \\ sin α & cos α & 0 \\ 0 & 0 & 1\end{matrix} )$$

The inverse of this rotation matrix is the transpose.

$${R}_{α}^{- 1} = {R}_{α}^{T} = ( \begin{matrix}cos α & sin α & 0 \\ - s in α & cos α & 0 \\ 0 & 0 & 1\end{matrix} )$$

$${( {a}_{α z} , {b}_{α z} , {c}_{α z} )}^{T} = {R}_{α}^{T} \times {( {a}_{T z} , {b}_{T z} , {c}_{T z} )}^{T}$$

Applying the rotation R_{α}^{T} removes the b_{α} component and aligns the beta (β) angle within X′-Z′ plane. This allows full circumferential angle determination of beta (β).

β_{T}=atan2(c_{αz},a_{αz})

a_{αz}=cos α*a_{Tz}+sin α*b_{Tz}

b_{αz}=−sin α*a_{Tz}+cos α*b_{Tz}=0

c_{αz}=c_{Tz}

To find gamma (γ) use alpha (α) and beta (β) to create a rotation matrix as follows:

$${R}_{α β} = ( \begin{matrix}cos α & - s in α & 0 \\ sin α & cos α & 0 \\ 0 & 0 & 1\end{matrix} ) ( \begin{matrix}cos β & 0 & sin β \\ 0 & 1 & 0 \\ - s in β & 0 & cos β\end{matrix} )$$

$${R}_{α β} = ( \begin{matrix}c α * c β & - s α & c α * s β \\ s α * c β & c α & s α * s β \\ - s β & 0 & c β\end{matrix} )$$

Remove the alpha (α) and beta (β) from the Y axis vector to solve for gamma (γ). This can be done by inverting the rotation matrix and multiply by Y axis unit vector. The inverse of this rotation matrix is the transpose.

$${R}_{αβ}^{- 1} = {R}_{αβ}^{T} = ( \begin{matrix}c α * c β & s α * c β & - s β \\ - s α & c α & 0 \\ c α * s β & s α * s β & c β\end{matrix} )$$

$${( {a}_{β x} , {b}_{β x} , {c}_{β x} )}^{T} = {R}_{z y}^{T} \times {( {a}_{T x} , {b}_{T x} , {c}_{T x} )}^{T}$$

This new Roll vector should have a zero in the c_{Tr} positions placing the vector on a Tip coordinate X-Y plane with values for a_{Tr} and b_{Tr}. Use these two values to determine gamma (γ) as follows:

γ_{T}=atan2(a_{βx},b_{βx})

a_{βx}=cos α*cos β*a_{Tx}+sin α*cosβ*b_{Tx}x−sin β*c_{Tx}

b_{βx}=−sin α*a_{Tx}+cos α*b_{Tx}

c_{βx}=cos α*sin β*a_{Tx}+sin α*sin β*b_{Tx}+cos β*c_{Tx}=0

Limiting Input Commands to Facilitate Solution:

As discussed in the previous section, this user input vector q is used to find the desired world space vector Q_{d} using the Transformation Matrix T_{0T}. The Q_{d} vector finds the desired coordinate values for X_{d}, Y_{d}, Z_{d}, [symbol] _{d}, and [symbol] _{d}.

Due to coordinate frame limitations, beta ( [symbol] ) should be greater than zero and less than 180 degrees. As beta approaches these limits, the Inverse Kinematics solver may become unstable. This instability is remediated by assigning a maximum and minimum value for beta that is higher than zero and lower than 180 degrees. How close to the limits depends on multiple variables and it is best to validate stability with assigned limits. For example, a suitable beta minimum may be 0.01 degrees and maximum 178 degrees.

The optimization scheme used to solve for the joint vector j (through the Inverse Kinematics) may become unstable with large changes in position and angles. While large displacements and orientation changes will often resolve, there are times when it may not. Limiting the position and angles change will help maintain mathematical stability. For large q changes, dividing the path into multiple steps may be helpful, optionally with a Trajectory planner. For reference, the maximum displacement per command may be set for 3.464 mm and maximum angle set for 2 degrees. The displacement is defined by the following:

Displacement=(x_{T}²+y_{T}²+z_{T}²)^{1/2}<2 mm
Angle=β_{T}<2 degrees

Segment Rotational Matrix:

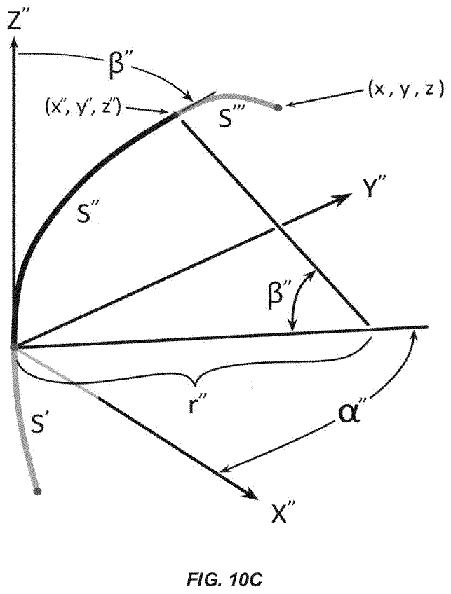

Referring now to FIG. 10A, solve rotation matrix

for an arc of a cord (no axial twist).

$$R = Joint Rotational matrix .$$

$$R = {R}_{z} \times {R}_{y} \times {R}_{z}^{T}$$

$${R}_{z} = ( \begin{matrix}cos ( α ) & - sin ( α ) & 0 \\ sin ( α ) & cos ( α ) & 0 \\ 0 & 0 & 1\end{matrix} ) about Z axis$$

$${R}_{y} = ( \begin{matrix}cos ( β ) & 0 & sin ( β ) \\ 0 & 1 & 0 \\ - sin ( β ) & 0 & cos ( β )\end{matrix} ) about Y axis$$

$${R}_{z y} = {R}_{z} \times {R}_{y} = ( \begin{matrix}cos ( α ) & - sin ( α ) & 0 \\ sin ( α ) & cos ( α ) & 0 \\ 0 & 0 & 1\end{matrix} ) \times ( \begin{matrix}cos ( β ) & 0 & sin ( β ) \\ 0 & 1 & 0 \\ - sin ( β ) & 0 & cos ( β )\end{matrix} )$$

$$R = {R}_{z y} \times {R}_{z}^{T} = ( \begin{matrix}c α * c β & - s α & c α * s β \\ s α * c β & c α & s α * s β \\ - s β & 0 & c β\end{matrix} ) \times ( \begin{matrix}c α & s α & 0 \\ - s α & c α & 0 \\ 0 & 0 & 1\end{matrix} )$$

$$R = ( \begin{matrix}c {α}^{2} * c β + s {α}^{2} & s α * c α * ( c β - 1 ) & c α * s β \\ s α * c α * ( c β - 1 ) & s {α}^{2} * c β + c {α}^{2} & s α * s β \\ - s β * c α & - s β * s α & c β\end{matrix} )$$

Segment Position Matrix:

Referring now to FIGS. 10B and 10C, solve position matrix for the cord

$$P = ( \begin{matrix}x \\ y \\ z\end{matrix} )$$

$$P = point in space relative to reference frame$$

$$r = S / β$$

$$x = r * c α * [ 1 - c β ]$$

$$x = ( S / β ) * c α * [ 1 - c β ]$$

$$y = ( S / β ) * c α * [ 1 - c β ]$$

$$z = ( S / β ) * s β$$

$$P = ( \begin{matrix}( S / β ) * c α * [ 1 - c β ] \\ ( S / β ) * s α * [ 1 - c β ] \\ ( S / β ) * s β\end{matrix} )$$

Segment Transformation Matrix:

Combine rotation and position matrix into a transformation matrix.

$$T = ( \begin{matrix}R & P \\ 0 & 1\end{matrix} )$$

$$T = ( \begin{matrix}c {α}^{2} * c β + s {α}^{2} & s α * c α * ( 1 - c β ) & c α * s β & ( S / β ) * c α * [ 1 - c β ] \\ s α * c α * ( 1 - c β ) & s {α}^{2} * c β + c {α}^{2} & s α * s β & ( S / β ) * s α * [ 1 - c β ] \\ - c α * s β & - s α * s β & c β & ( S / β ) * s β \\ 0 & 0 & 0 & 1\end{matrix} )$$

### Compound Lateral Stiffness Transformation Matrix:

For segments with an instruments tip passing through, or having instruments within with changing lateral stiffness, or when the segment is transitioning out the distal end of a guide sheath, the Transformation Matrix requires partial Transformation matrix within the length of the Segment. In the drawing to the right these partial Transformational Matrices are identified within the length of the Segment with prime marks (as seen in the drawing to the right) illustrated here is a segment with three distinct sections, S′, S″ and S′″ which are all sub sections of one segment S. Note that the sum of the lengths of S primes equals the total length of the segment.

S=S′+S″+S′″

To resolve the full segment Transformation Matrix, partial transformation matrix is required for each subsection (with different lateral stiffness) of the segment. The subsection Transformation Matrix are mathematically similar to the homogenous (lateral stiffness) Segment Transformation above, but with partial values as indicated below such that T′ can be resolved as follows:

$$( \begin{matrix}c {α}^{′2} * c {β}^{′} + s {α}^{′2} & s {α}^{′} * c {α}^{′} * ( 1 - c {β}^{′} ) & c {α}^{′} * s {β}^{′} & ( {S}^{′} / {β}^{′} ) * c {α}^{′} * [ 1 - c {β}^{′} ] \\ s {α}^{′} * c {α}^{′} * ( 1 - c {β}^{′} ) & s {α}^{′2} * c {β}^{′} + c {α}^{′2} & s {α}^{′} * s {β}^{′} & ( {S}^{′} / {β}^{′} ) * s {α}^{′} * [ 1 - c {β}^{′} ] \\ - c {α}^{′} * s {β}^{′} & - s {α}^{′} * s {β}^{′} & c {β}^{′} & ( {S}^{′} / {β}^{′} ) * s {β}^{′} \\ 0 & 0 & 0 & 1\end{matrix} )$$

These Segment subsection Matrix are multiplied to resolve the full segment Transformation Matrix.

T=T′×T″×T′″× . . . Tⁿ′

Note that the first subsection Rotation Matrix orients the lateral bend direction and subsequent subsection Rotation Matrix bend on the same plan (though at a different rates). Therefore, the first partial segment Rotation Matrix sets the direction and the rest have an alpha with a value of zero.

α′=α; α″=α′″=αⁿ′=0

Rotational Matrix Generalized:

Rᵢ indicates the rotation of a reference frame attached to the tip of segment “i” relative to a reference frame attached to its base (or the tip of Segment “i-1”).

Rᵢ=R(αᵢ,βᵢ,Sᵢ); i=0,1,2, . . . n;

i=0, sensor readings to input by manual (rotation & axial motion) actuation of the catheter proximal of the first segment.
i=1, is the most proximal segment.
i=n, the most distal segment (which is 2 for a two-segment catheter).

System Position Generalized:

Pᵢ indicates the origin of the distal end of segment “i” relative to a reference frame attached to its base (or the tip of Segment “i-1”).

$$P = ( \begin{matrix}{x}_{i} \\ {y}_{i} \\ {z}_{i}\end{matrix} )$$

Continuum Translation Matrix:

Tᵢ is the transformation matrix from a frame at the distal end of segment “i” to a frame attached to its base (or the tip of Segment “i-1”).

$${T}_{i} = ( \begin{matrix}{R}_{i} & {P}_{i} \\ 0 & 1\end{matrix} ) , for i from 1 to n .$$

$${T}_{i} = ( \begin{matrix}c {α}_{i}^{2} * c β i + s {α}_{i}^{2} & s {α}_{i} * c {α}_{i} * ( 1 - c {β}_{i} ) & c {α}_{i} * s {β}_{i} & ( {S}_{i} / {β}_{i} ) * c {α}_{i} * [ 1 - c {β}_{i} ] \\ s {α}_{i} * c {α}_{i} * ( 1 - c {β}_{i} ) & s {α}_{i}^{2} * c {β}_{i} + c {α}_{i}^{2} & s {α}_{i} * s {β}_{i} & ( {S}_{i} / {β}_{i} ) * s {α}_{i} * [ 1 - c {β}_{i} ] \\ - c {α}_{i} * s {β}_{i} & - s {α}_{i} * s {β}_{i} & c {β}_{i} & ( {S}_{i} / {β}_{i} ) * s {β}_{i} \\ 0 & 0 & 0 & 1\end{matrix} )$$

T_{w} is the transformation matrix from the most distal segment's tip reference frame to the world reference frame which is located proximal to the manually (versus fluidically) driven joints.

T_{w}=T₀×T₁×T₂× . . . Tⁿ

Use this matrix to solve the Forward Kinematics for current tip position P_{w} and axial unit vector V_{wz}.

P_{w}=T_{w}*(0,0,0,1)

P_{w}=(x_{w},y_{w},z_{w})

V_{wz}=T_{w}*(0,0,1,0)

Vwz=(a_{wz},b_{wz},c_{wz})

Combine for World Space Tip Position:

Solve tip world space Q_{W}, by combining P_{w} and V_{wz} as follows.

Q_{W}=(X_{W},Y_{W},Z_{W},α_{w},β_{w})

X_{W}=x_{w}=xₙ

Y_{W}=y_{w}=yₙ

Z_{W}=z_{w}=zₙ

### Solve for Segment Angles:

α_{w}=atan2(a_{wz},b_{wz})

if |a_{wz}|<min; than a_{wz}=(a_{wz}/|a_{wz}|)*min

### Current β_{w}

β_{w}=atan2(c_{wz},ab_{wz})

ab_{wz}=(a_{wz}²+b_{wz}²)^{1/2}

if |c_{wz}|<min; than c_{wz}=(c_{wz}/|c_{wz}|*min

New β_{w} and γ_{w}

β_{w}=atan2(c_{wz},a_{αz})

a_{αz}=cos α*a_{wz}+sin α*b_{wz}

if |c_{wz}|<min; than c_{wz}=(c_{wz}/|c_{wz}|)*min

γ_{w}=atan2(aβx,b_{βx})

a_{βx}=cos α*cos β*a_{wx}+sin α*cos β*b_{wx}−sin β*c_{wx}

b_{βx}=−sin α*a_{wx}+cos α*b_{wx}

if |a_{βx}|<min; than a_{βx}=(a_{βx}/|a_{βx}|)*min

Convert Q_{W} to Q_{J} for use with Jacobian

B_{Wx}=β_{w}*cos(α_{w})

B_{Wy}=β_{w}*sin(α_{w})

Q_{J}=(X_{W},Y_{W},Z_{W},β_{Wx},β_{Wy})

Numerical Jacobian:

To solve the unique Q_{J} for a deviation in joint variable (αᵢ, βᵢ, Sᵢ), one at a time, deviate each variable in every Segment by using the Transformation matrix. Then combine the resultant Q_{J} vectors to form a numeric Jacobian. By using small single variable deviations from the current joint space, a localized Jacobean can be obtained. This Jacobean can be used in several ways to iteratively find a solution for segment joint variables to a desired world space position, Q_{d}. Preferable the Jacobean is invertible in which case the difference vector between the current and desired world position can be multiplied by the inverse Jacobian J⁻¹ to iteratively approach a correct joint space variables. Repeat this process until the Forward Kinematics calculates a position vector equivalent to Q_{d} within a minimum error. Check Joint Space results (αᵢ, βᵢ, Sᵢ for i=0, 1, n) for accuracy, solvability, workspace limitations and errors.

J⁻¹*J=I

αᵢ>=−360° & αᵢ<=360°

βᵢ>βₘᵢₙ & βᵢ<βₘₐₓ

βᵢ≈0° or 180° (or βₘₐₓ)

β_{w}≈0° or 180°

Sᵢ>Sₘᵢₙ; Sᵢ<Sₘₐₓ

A more stable Jacobian may optionally be achieved by altering αᵢ, βᵢ, Sᵢ, to be βx₁, β_{y}i, Sᵢ, where:

βₓᵢ=βᵢ*cos(αᵢ)

β_{yi}=βᵢ*sin(αᵢ).

To convert back:

αᵢ=atan2(βₓᵢ,β_{yi})

βᵢ=(βₓᵢ²+β_{yi}²)^{1/2}

Note that αᵢ defines the direction that the segment is pointing in the world X-Y plane and when pointing in the direction of the Z axis, αᵢ becomes undefined. Also, βᵢ, being the middle angle in a gimble sequence of angles, may be limited to a range of 180° and always positive, which works well for the Pi covert back equation.

βₓᵢ and β_{yi} values are always definable and using radians range between positive and negative Pi. Unlike αᵢ and Pi, βxᵢ and βyᵢ both equal zero and us defined when the segment points in the Z direction. In addition, αᵢ has a numerical step when transitioning from 359 to zero degrees which is more challenging to manage that the smooth transitions of βxᵢ and βyᵢ.

### Target and Achievable:

With more input variables than target variables, the pseudo Jacobean will solve for a solution through the least squares fit. There may be more than one solution and any given solution target may be achieved while being beyond the input variable range of motion. Using several techniques workspace can be expanded and often a target may be achieved within the limits of the input variables range. There are at least three techniques that can be used within the interactive equations which are herein referred to as Scaling, Shifting, and Skewing, and these or other techniques can be used alone or in any combinations of two, three, or more.

The input for the robot can be used to change the pressure. The pressure range may have a minimum and a maximum, optionally 0 to 500 PSI or more. When a numerical solution produces a set of pressures that have some lumens outside the range, the solution may not be achievable.

Changing equal amounts of pressure within each of the three lumens of a Segment may cause a displacement change, while retaining orientation. Therefore, if 10 PSI is removed from Pr_{1A}, Pr_{1B}, and Pr_{1C}, the orientation should remain the same and only the position will change. Shifting changes the pressure within a Segment by equal amounts in order to bring all three pressures within range. The consequence is a translation along an axis between the Segments origin and the Segments tip.

When a Segments pressure range between the lumens is larger than the available pressure range of the source, a process of Scaling occurs to proportionally reduce the three-lumen pressure so they will fit, after Shifting, within the minimum and maximum pressures available.

With a segment lumen pressures may be scaled and/or shifted; the Jacobean tends to achieve a solution that meets the Target Q variables but may also remain slightly shifted outside the pressures range. Skewing nudges the Segment towards being within the range available pressure while maintaining an accurate target output. A small multiple bias the Segment towards either higher or lower pressure. This skewing multiple (ζ) attempts to shift a segment towards being inside the pressure boundary while moving neighboring segments to compensate. The result is a tendency for the segments to gravitate towards an achievable solution. The multiple is only applied when the three-lumen pressures bandwidth is within the available pressure range, and when one segment is outside the pressure range, or two segments are out in complementary directions. Complementary directions in a two Segment system would have one Segment below minimum pressure and the other Segment above maximum pressure. The skewing multiple (ζ) may be applied to modify pressure, or to the segment tri-lengths. Both cases were evaluated and applying the multiple to the tri-lengths (post Shifting and Scaling) appears to optimize a bit quicker. The multiple ζ works well with the Segment tri-lengths (S) at about 2% (1.02 and 0.098), and with the pressure (Pr) at 9% (1.09 and 0.091).

| | |
|---|---|
| S_{1A} = S_{1A} * ζ | S_{2A} = S_{2A}/ζ |
| S_{1B} = S_{1B} * ζ | S_{2B} = S_{2B}/ζ |
| S_{1C} = S_{1C} * ζ | S_{2C} = S_{2C}/ζ |

```
Skew = .98
Test1 = IF( SEG₁Pr_{MAX} & SEG₂PR_{MAX} > Pr_{MAX}, 1,
      IF( SEG₁Pr_{MIN} & SEG₂PR_{MIN} < Pr_{MIN}, −1, 0 )
      )
Test2 = IF( Test1 = 0,
      IF( SEG₁Pr_{MAX} OR SEG₂PR_{MAX} > Pr_{MAX}, 1,
      IF( SEG₁Pr_{MIN} OR SEG₂PR_{MIN} < Pr_{MIN}, −1, 0 )
      ), 0)
ζ = IF(Test2 = 1, Skew, IF(Test2 = −1, 1/Skew, 1))
```

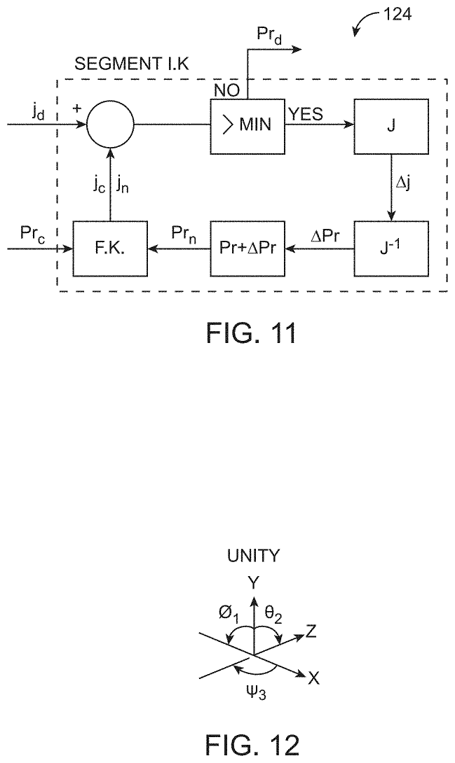

Referring now to FIG. 11, a lumen pressure flow chart 120 is shown that can be used by a processor of the robotic system to determine pressures associated with the desired movement or position of an actual or virtual catheter.

Solving for Segment Balloon Array End Coordinates:

A balloon array is a set of fluidically connected balloons along one side of a segment. Find end coordinates for each balloon array within a segment base frame. Assume balloon arrays are spaced at 120 degrees apart around the cordial axis, that the first is located on the X axis, and that the array balloons remain axially aligned through the length of the segment.

r_{A}=radius of balloon center in balloon array from cordial axis
θ=angular period of balloons within segment (120° for a 3 array segment)
θ_{A}=0; θ_{B}=120; θ_{C}=240
Arc Start points for balloon arrays:

$${P}_{0 A} = ( \begin{matrix}{r}_{A} * cos ( {θ}_{A} ) \\ {r}_{A} * sin ( {θ}_{A} ) \\ 0 \\ 1\end{matrix} ) ,$$

$${P}_{0 B} = ( \begin{matrix}{r}_{A} * cos ( {θ}_{B} ) \\ {r}_{A} * sin ( {θ}_{B} ) \\ 0 \\ 1\end{matrix} ) ,$$

$${P}_{0 C} = ( \begin{matrix}{r}_{A} * cos ( {θ}_{C} ) \\ {r}_{A} * sin ( {θ}_{C} ) \\ 0 \\ 1\end{matrix} )$$

Arc unit vectors for axial orientation of balloon arrays are as follows:

$${V}_{0 A} = ( \begin{matrix}0 \\ 0 \\ 1 \\ 0\end{matrix} ) , {V}_{0 B} = ( \begin{matrix}0 \\ 0 \\ 1 \\ 0\end{matrix} ) , {V}_{0 C} = ( \begin{matrix}0 \\ 0 \\ 1 \\ 0\end{matrix} ) ,$$

Use local (for one Segment) Transformation Matrix to find Balloon Array distal end coordinates.

$$T = ( \begin{matrix}c {α}^{2} * c β + s {α}^{2} & s α * c α * ( 1 - c β ) & c α * s β & ( S / β ) * c α * [ 1 - c β ] \\ s α * c α * ( 1 - c β ) & s {α}^{2} * c β + c {α}^{2} & s α * s β & ( S / β ) * s α * [ 1 - c β ] \\ - c α * s β & - s α * s β & c β & ( S / β ) * s β \\ 0 & 0 & 0 & 1\end{matrix} )$$

For segments with multiple lateral stiffnesses: T=T′×T″×T′″× . . . Tⁿ′

Pᵢ₁=T×P₀

P_{1A}=T×P_{0A}

P_{1B}=T×P_{0B}

P_{1C}=T×P_{0C}

For each Balloon Array set the origin at the starting points to normalize distal endpoint coordinates dP. This is helpful for solving for the Balloon Array arc, S, which follows in the Find Array Arc Lengths section below.

dPᵢ=Pᵢ₁−Pᵢ₀=(x₀,y₀,z₀)Segment Centerline Cord

dP_{A}=P_{A1}−P_{A0}=(x_{A},y_{A},z_{A}) Balloon Cord A

dP_{B}=P_{B1}−P_{B0}=(x_{B},y_{B},z_{B}) Balloon Cord B

dP_{C}=P_{C1}−P_{C0}=(x_{C},y_{C},z_{C}) Balloon Cord C

All cordial orientation vectors are equivalent.

V=(a_{iz},b_{iz},c_{iz}), for i from 1 to n.

Find Array Arc Lengths:

Referring again to FIGS. 10B and 10C, solve for general Balloon Array cord lengths and radii as follows.

(α, β, S), α represent the bend direction (about z axis starting at x axis), β the bend amount (off the z axis), and S the length of an arc anchored to the origin of a reference frame.
(x, y, z) is the coordinate location of a point at the end of the arc.
r is the balloon array cord radius.

$$h = {( {x}^{2} + {y}^{2} )}^{1 / 2}$$

$$r = ( {h}^{2} + {z}^{2} ) / ( 2 * h )$$

$$r - h = ( {z}^{2} - {h}^{2} ) / ( 2 * h )$$

$$α = atan 2 ( x , y )$$

$$β = atan 2 ( z , r - h )$$

$$S = r * β$$

$$( \begin{matrix}α \\ β \\ S\end{matrix} ) = ( \begin{matrix}\begin{matrix}atan 2 ( x , y ) \\ atan 2 ( r - h , z )\end{matrix} \\ r * atan 2 ( r - h , z )\end{matrix} )$$

$${S}_{i} = ( {h}_{i}^{2} + {z}_{i}^{2} ) / ( 2 * {h}_{i} ) * β , {h}_{i} = {( {x}_{i}^{2} + {y}_{i}^{2} )}^{1 / 2}$$

i (segment center cord)
Solve S for cords A, B, C, (segment center cord)
Note that segment center cord (S, β, α) is already determined.

### When β>βₘᵢₙ

Sₐ=(h_{A}²+z_{A}²)/(2*h_{A})*β, h_{A=(x}_{A}²+y_{A}²)^{1/2}

S_{B}=(h_{B}²+z_{B}²)/(2*h_{B})*β, h_{B}=(x_{B}²+y_{B}²)^{1/2}

S_{C}=(h_{C}²+z_{C}²)/(^{2*}h_{C})*β, h_{C}=(x_{C}²+y_{C}²)^{1/2}

When β=<βₘᵢₙ

S_{A}=S_{B}=S_{C}=Sᵢ

For segments with multiple lateral stiffness' where T=T′×T″×T′″× . . . Tⁿ′, The process above is resolved for each segment partial section and the sub-Segment lengths (S′, S″, . . . S′ⁿ) are summed for each cord length (A, B, and C). Similar math is used for the subsection lengths.
When β′>βₘᵢₙ (may not be needed)

S′_{A}=(h′_{A}²+z′_{A}²)/(2*h′_{A})*β′, h′_{A}=(x′_{A}²+y′_{A}²)^{1/2}

S′_{B}=(h′_{B}²+z′_{B}²)/(2*h′_{B})*β′, h′_{B}=(x′_{B}²+y′_{B}²)^{1/2}

S′_{C}=(h′_{C}²+z′_{C}²)/(2*h′_{C})*β′, h′_{C}=(x′_{C}²+y′_{C}²)^{1/2}
When β′=<βₘᵢₙ (may not be needed)

S′_{A}=S′_{B}=S′_{C}=S′ᵢ

Repeat for each subsegment section and sum partial lengths

S_{A}=S_{A}′+S_{A}″+ . . . S_{A}ⁿ′

S_{B}=S_{B}′S_{B}″+ . . . S_{B}ⁿ′

S_{C}=S_{C}′+S_{C}″+ . . . S_{C}ⁿ′

Segment Internal Load Conditions:

Segment spring force may be proportional to a spring rate with extension.

F_{S}=K_{F}*Sᵢ+F₀

Where F is the sum of the balloon forces, K_{F} is the spring constant, and F₀ is the offset force.

F₀=F_{preload}−K_{F}*Sₘᵢₙ

Where F_{preload} is the preload force at the minimum segment length Sₘᵢₙ.
Sum balloon array forces as follows:

F_{Pr}=F_{A}+F_{B}+F_{C}=A*(Pr_{A}+Pr_{B}+Pr_{C})

F_{S}=F_{Pr}

Sᵢ=(A*(Pr_{A}+Pr_{B}+Pr_{C})−F₀)/K_{F}

Segment spring torque proportional to a spring rate with bend angle.

M_{S}=K_{M}*β+M₀

β=(M_{S}−M₀)/K_{M}

Where M is the internal moment applied to the Segment, K_{M} is a angular spring constant can, and M₀ is the preload moment (compensation for no load bend).
Sum of balloon array torques:

$${r}_{A} = radius of balloon center in balloon array from cordial axis$$

$$θ = angular period of balloons within segment ( 120 ° for a 3 array setment )$$

$${θ}_{A} = θ° ; {θ}_{B} = 120 ° ; {θ}_{C} = 240 °$$

$$\begin{matrix}{M}_{x} = {F}_{A} * {r}_{A} * sin ( {θ}_{A} ) + {F}_{B} * {r}_{A} * sin ( {θ}_{B} ) + {F}_{C} * {r}_{A} * sin ( {θ}_{C} ) \\ = ( {3}^{1 / 2} / 2 ) * A * {r}_{A} * ( {Pr}_{B} - {Pr}_{C} )\end{matrix}$$

$$\begin{matrix}{M}_{y} = - {F}_{A} * {r}_{A} * cos ( {θ}_{A} ) - {F}_{B} * {r}_{A} * cos ( {θ}_{B} ) - {F}_{C} * {r}_{A} * cos ( {θ}_{C} ) \\ = ( 1 / 2 ) * A * {r}_{A} * ( {Pr}_{B} + {Pr}_{C} - 2 * {Pr}_{A} )\end{matrix}$$

$$\begin{matrix}{M}_{s} = {( {M}_{x}^{2} + {M}_{y}^{2} )}^{1 / 2} \\ = ( {( ( {3}^{1 / 2} / 2 ) * A * {r}_{A} * ( {Pr}_{B} - {Pr}_{C} ) )}^{2} + ( ( 1 / 2 ) * A * {r}_{A} * ( {Pr}_{B} + {Pr}_{C} - \\ {{2 * {Pr}_{A} ) )}^{2} )}^{1 / 2} \\ = ( A / 2 ) * {r}_{A} * {( 3 * {( {Pr}_{B} - {Pr}_{C} )}^{2} + {( {Pr}_{B} + {Pr}_{C} - 2 * {Pr}_{A} )}^{2} )}^{1 / 2} \\ = ( A / 2 ) * {r}_{A} * ( ( 3 * {Pr}_{B}^{2} - 6 * {Pr}_{B} * {Pr}_{C} + 3 * {Pr}_{C}^{2} ) + \\ ( {Pr}_{B}^{2} + {Pr}_{B} * {Pr}_{C} - 2 * {Pr}_{A} * {Pr}_{B} + {Pr}_{B} * {Pr}_{C} + {Pr}_{C}^{2} - \\ {2 * {Pr}_{A} * {Pr}_{C} - 2 * {Pr}_{A} * {Pr}_{B} - 2 * {Pr}_{A} * {Pr}_{C} + 4 * {Pr}_{A}^{2} ) )}^{1 / 2} \\ = ( A / 2 ) * {r}_{A} * ( 4 * {Pr}_{B}^{2} - 4 * {Pr}_{B} * {Pr}_{C} + 4 * {Pr}_{C}^{2} - 4 * {Pr}_{A} * {Pr}_{C} + \\ {4 * {Pr}_{A}^{2} )}^{1 / 2} \\ = A * {r}_{A} * {( {Pr}_{B}^{2} - {Pr}_{B} * {Pr}_{C} + {Pr}_{C}^{2} - {Pr}_{A} * {Pr}_{B} - {Pr}_{A} * {Pr}_{C} + {Pr}_{A}^{2} )}^{1 / 2} \\ = A * {r}_{A} * ( {Pr}_{A}^{2} + {Pr}_{B}^{2} + {Pr}_{C}^{2} - {Pr}_{A} * {Pr}_{B} - {Pr}_{B} * {Pr}_{C} - {Pr}_{C} - \\ {{Pr}_{C} * {Pr}_{A} )}^{1 / 2}\end{matrix}$$

$${M}_{Pr} = {M}_{s}$$

$$β = [ A * {r}_{A} * {( {Pr}_{A}^{2} + {Pr}_{B}^{2} + {Pr}_{C}^{2} - {Pr}_{A} * {Pr}_{B} - {Pr}_{B} * {Pr}_{C} - {Pr}_{C} * {Pr}_{A} )}^{1 / 2} - {M}_{0} ] / {K}_{M}$$

Moment direction angle (α):

$${M}_{x} = ( {3}^{1 / 2} / 2 ) * A * {r}_{A} * ( {Pr}_{B} - {Pr}_{C} )$$

$${M}_{y} = ( 1 / 2 ) * A * {r}_{A} * ( {Pr}_{B} + {Pr}_{C} - 2 * {Pr}_{A} )$$

$$cos ( α ) = {M}_{y} / {M}_{S}$$

$$sin ( α ) = - {M}_{x} / {M}_{S}$$

$$β = ( {M}_{S} - {M}_{0} ) / {K}_{M}$$

$$\begin{matrix}{β}_{x} = [ ( {M}_{S} - {M}_{0} ) / {K}_{M} ] * cos ( α ) \\ = [ ( {M}_{S} - {M}_{0} ) / {K}_{M} ] * {M}_{y} / {M}_{S} \\ = [ 1 - ( {M}_{0} / {M}_{S} ) ] * {M}_{y} / {K}_{M} \\ = [ 1 - ( {M}_{0} / A * {r}_{A} * ( {Pr}_{A}^{2} + {Pr}_{B}^{2} + {Pr}_{C}^{2} - {Pr}_{A} * {Pr}_{B} - {Pr}_{B} * {Pr}_{C} - \\ {{Pr}_{C} * {Pr}_{A} )}^{1 / 2} ) ] * ( 1 / 2 ) * A * {r}_{A} * ( {Pr}_{B} + {Pr}_{C} - 2 * {Pr}_{A} ) / {K}_{M}\end{matrix}$$

$$\begin{matrix}{β}_{y} = - [ ( {M}_{S} - {M}_{0} ) / {K}_{M} ] * sin ( α ) \\ = - [ ( {M}_{S} - {M}_{0} ) / {K}_{M} ] * {M}_{x} / {M}_{S} \\ = - [ 1 - ( {M}_{0} / {M}_{S} ) ] * {M}_{x} / {K}_{M} \\ = - [ 1 - ( {M}_{0} / A * {r}_{A} * ( {Pr}_{A}^{2} + {Pr}_{B}^{2} + {Pr}_{C}^{2} - {Pr}_{A} * {Pr}_{B} - {Pr}_{B} * {Pr}_{C} - \\ {{Pr}_{C} - {Pr}_{C} * {Pr}_{A} )}^{1 / 2} ) ) ] * ( {3}^{1 / 2} / 2 ) * A * {r}_{A} * ( {Pr}_{B} - {Pr}_{C} ) / {K}_{M}\end{matrix}$$

### With M₀=0,

βₓ=(1/2)*A*r_{A}*(Pr_{B}+Pr_{C}−2*Pr_{A})/K_{M}

β_{y}=−(3^{1/2}/2)*A*r_{A}*(Pr_{B}−Pr_{C})/K_{M}

α=atan2(βₓ, β_{y}); Note the minus sign for Mx is applied for matching direction.

α=atan2((Pr_{B}+Pr_{C}−2*Pr_{A}),−1.73205*(Pr_{B}−Pr_{C}))

if(|Pr_{B}−Pr_{C}|<min)&(|Pr_{B}+Pr_{C}−2*Pr_{A}|<min); than α=0

Solve for P_{A}, P_{B}, and P_{C} using the following three equations and a Jacobian. Pressures should meet these conditions to solve.

|Pr_{A}−Pr_{B}|+|Pr_{B}−Pr_{C}|+|Pr_{C}−Pr_{A}|<MIN_{DIfference}; related to |β_{Calc}|>βₘᵢₙ

Pr_{A},Pr_{B},Pr_{C}>Min_{Pressure}

### Setting up Input α_{Desired} for Jacobian

IF(α_{Desired}>180°,α_{Desired}−360°, IF(α_{Desired}<−180°,α_{Desired}+360°,α_{Desired}))

Segment or joint position may be found with lumen pressures as follows:

S_{Calc}=[A*(Pr_{A}+Pr_{B}+Pr_{C})−F₀]/K_{F}

βₓ=(1/2)*A*r_{A}*(Pr_{B}+Pr_{C}−2*Pr_{A})/K_{M}

β_{y}−(3^{1/2}/2)*A*r_{A}*(Pr_{B}−Pr_{C})/K_{M}

and:

β_{Calc}=[A*r_{A}*(Pr_{A}²+Pn_{B}²+Pr_{C}²−Pr_{A}*Pr_{B}−Pr_{B}*Pr_{C}−Pr_{C}*Pr_{A})^{1/2}]/K_{M}

α_{Calc}=atan2((Pr_{B}+Pr_{C}−2*Pr_{A}),−3^{1/2}*(Pr_{B}−Pr_{C}))

IF(|α_{Calc}|>45° AND|α_{Calc}|<315°,

Note that:

IFα_{Calc}>0°,IF(α_{Desired}<0°,α_{Desired}+360°,α_{Desired}),

IF(α_{Desired}>0°,α_{Desired}−360°,α_{Desired})),α_{Desired})

For segments with multiple lateral stiffness lengths, we can solve for a resultant bend constant by adding the contributions of the subsegments. To avoid dividing by a zero length solve for the inverse of the bend constant.

dK_{M}′⁻¹=(S′/S)*(1/K_{M}′)

ΣK_{M}⁻¹=(1/S)*[(S′/K_{M}′)+(S″/K_{M}″)+ . . . (Sn′/K_{M}ⁿ′)]

ΣK_{M}=S/[(S′/K_{M}′)+(S″/K_{M}″)+ . . . (Sn′/K_{M}ⁿ′)]

The Lumen pressures for segments with multiple lateral stiffnesses can be solved as follows:

S_{Calc}=[A*(Pr_{A}+Pr_{B}+Pr_{C})−F₀]/K_{F}

βₓ=(1/2)*A*r_{A}*(Pr_{C}+Pr_{B}−2*Pr_{A})/ΣK_{M}

β_{y}=(3^{1/2}/2)*A*r_{A}*(Pr_{C}−Pr_{B})/ΣK_{M}

Calculating Segment Multiple Section Spring Constants

To solve the K_{M}ⁿ′ constants above we can start with the component bend constants. Referring to FIG. 11A, bend constants are assigned to various components of the Catheter Assembly. As shown the Guide and Catheter overlap and the Instrument Body and runs through the Segments, Catheter and Guide. The lateral bend spring constants (K_{M}ⁿ′) is a combination of these component bend constants and a change in lateral stiffness is associated with a change of components along the length of a segment.

For instance, for Segment 1 K_{M}′ with the Guide overlapping would have the following

K_{M}′=1/[(1/K_{M.S1})+(1/K_{M.Guide})]

Add the Instrument Body through it and K_{M}′ becomes:

K_{M}′=1/[(1/K_{M.S1})+(1/K_{M.Guide})+(1/K_{M.Instr-B})]

If the Guide ends and Instrument Body continues mid-way along S1, K_{M}″ is as follows:

K_{M}″=1/[(1/K_{M.S1})+(1/K_{M.Instr-B})]

If more distal along S1 the Instrument Body becomes the Instrument Tip, K_{M}′″ is added and can be found as follows:

K_{M}′″=1/[(1/K_{M.S1})+(1/K_{M.Instr-T})]

If prior to the end of S1 the Instrument Tip ends a fourth constant is introduced as, KM″″ and how with nothing passing through this portion of the Segment becomes as follows:

K_{M}′″=1/[(1/K_{M.S1})]=K_{M.S1}

Note that each KM represents the spring constant for the whole length of a Segment. To calculate the relative bend for each length the constants are normalized with the length relative to the Segment S value as indicated above.

Find lumen pressures from joint variables:

$${β}_{y} = - ( {3}^{1 / 2} / 2 ) * A * {r}_{A} * ( {Pr}_{B} - {Pr}_{C} ) / {K}_{M}$$

$${Pr}_{C} = {Pr}_{B} + 2 * ( {β}_{y} / {3}^{1 / 2} ) * {K}_{M} / ( A * {r}_{A} )$$

$${β}_{x} = ( 1 / 2 ) * A * {r}_{A} * ( {Pr}_{B} + {Pr}_{C} - 2 * {Pr}_{A} ) / {K}_{M}$$

$$2 * {β}_{x} * {K}_{M} / ( A * {r}_{A} ) = {Pr}_{B} + {Pr}_{B} + 2 * ( {β}_{y} / {3}^{1 / 2} ) * {K}_{M} / ( A * {r}_{A} ) - 2 * {Pr}_{A}$$

$$2 * {β}_{x} * {K}_{M} / ( A * {r}_{A} ) + 2 * {Pr}_{A} = 2 * {Pr}_{B} + 2 * ( {β}_{y} / {3}^{1 / 2} ) * {K}_{M} / ( A * {r}_{A} )$$

$${Pr}_{B} = {Pr}_{A} + {β}_{x} * {K}_{M} / ( A * {r}_{A} ) - ( {β}_{y} / {3}^{1 / 2} ) * {K}_{M} / ( A * {r}_{A} )$$

$${Pr}_{B} = {Pr}_{A} + ( {β}_{x} - {β}_{y} / {3}^{1 / 2} ) * {K}_{M} / ( A * {r}_{A} )$$

$${S}_{Calc} = [ A * ( {Pr}_{A} + {Pr}_{B} + {Pr}_{C} ) - {F}_{0} ] / {K}_{F}$$

$$( {S}_{Calc} * {K}_{F} + {F}_{0} ) / A = {Pr}_{A} + {Pr}_{B} + {Pr}_{B} + 2 * ( {β}_{y} / {3}^{1 / 2} ) * {K}_{M} / ( A * {r}_{A} )$$

$$( {S}_{Calc} * {K}_{F} + {F}_{0} ) / A = {Pr}_{A} + 2 * [ {Pr}_{A} + ( {β}_{x} - {β}_{y} / {3}^{1 / 2} ) * {K}_{M} / ( A * {r}_{A} ) ] + 2 * ( {β}_{y} / {3}^{1 / 2} ) * {K}_{M} / ( A * {r}_{A} )$$

$$( {S}_{Calc} * {K}_{F} + {F}_{0} ) / A = 3 * {Pr}_{A} + [ 2 * ( {β}_{x} - {β}_{y} / {3}^{1 / 2} ) + 2 * ( {β}_{y} / {3}^{1 / 2} ) ] * {K}_{M} / ( A * {r}_{A} )$$

$$( {S}_{Calc} * {K}_{F} + {F}_{0} ) / A = 3 * {Pr}_{A} + 2 * {β}_{x} * {K}_{M} / ( A * {r}_{A} )$$

$${Pr}_{A} = ( {S}_{Calc} * {K}_{F} + {F}_{0} ) / ( 3 * A ) - 2 * {β}_{x} * {K}_{M} / ( 3 * A * {r}_{A} )$$

$$\begin{matrix}{Pr}_{B} = {Pr}_{A} + ( {β}_{x} - {β}_{y} / {3}^{1 / 2} ) * {K}_{M} / ( A * {r}_{A} ) \\ = ( {S}_{Calc} * {K}_{F} + {F}_{0} ) / ( 3 * A ) - 2 * {β}_{x} * {K}_{M} / ( 3 * A * {r}_{A} ) + \underline{3 *} \\ ( {β}_{x} - {β}_{y} / {3}^{1 / 2} ) * {K}_{M} / ( \underline{3 *} A * {r}_{A} )\end{matrix}$$

$${Pr}_{B} = ( {S}_{Calc} * {K}_{F} + {F}_{0} ) / ( 3 * A ) + ( {β}_{x} - 3 * {β}_{y} / {3}^{1 / 2} ) * {K}_{M} / ( 3 * A * {r}_{A} )$$

$${Pr}_{C} = {Pr}_{B} + 2 * ( {β}_{y} / {3}^{1 / 2} ) * {K}_{M} / ( A * {r}_{A} )$$

$${Pr}_{C} = ( {S}_{Calc} * {K}_{F} + {F}_{0} ) / ( 3 * A ) + ( {β}_{x} - 3 * {β}_{y} / {3}^{1 / 2} ) * {K}_{M} / ( 3 * A * {r}_{A} ) + \underline{6 *} ( {β}_{y} / {3}^{1 / 2} ) * {K}_{M} / ( \underline{3 *} A * {r}_{A} )$$

$${Pr}_{C} = ( {S}_{Calc} * {K}_{F} + {F}_{0} ) / ( 3 * A ) + ( {β}_{x} + 3 * {β}_{y} / {3}^{1 / 2} ) * {K}_{M} / ( 3 * A * {r}_{A} )$$

Lumen Pressures from Joint Variables

Pr_{A}=(S_{Calc}*K_{F}+F₀)/(3*A)−2*βₓ*K_{M}/(3*A*rA)

Pr_{B}=(S_{Calc}*K_{F}+F₀)/(3*A)+(βₓ−3*β_{y}/3^{1/2})*K_{M}/(3*A*r_{A})

Pr_{C}=(S_{Calc}*K_{F}+F₀)/(3*A)+(βₓ+3*β_{y}/3^{1/2})*K_{M}/(3*A*r_{A})

For segments with multiple lateral stiffness lengths, solve for a resultant bend constant by adding the contributions of the subsegments. The sub constants are scaled by the section's relative length to the whole segment. To avoid dividing by a zero length solve for the inverse of the bend constant.

dK_{M}′⁻¹=(S′/S)*(1/K_{M}′)

ΣK_{M}⁻¹=(1/S)*[(S′/K_{M}′)+(S″/K_{M}″)+ . . . (Sn′/K_{M}ⁿ′)]

ΣK_{M}=S/[(S′/K_{M}′)+(S″/K_{M}″)+ . . . (Sn′/K_{M}ⁿ′)]

The Lumen pressures for segments with multiple lateral stiffnesses can be solved as follows:

Pr_{A}=(S_{Calc}*K_{F}+F₀)/(3*A)−2*βₓ*ΣK_{M}/(3*A*r_{A})

Pr_{B}=(S_{Calc}*K_{F}+F₀)/(3*A)+(βₓ−3*β_{y}/3^{1/2})*ΣK_{M}/(3*A*r_{A})

Pr_{C}=(S_{Calc}*K_{F}+F₀)/(3*A)+(βₓ+3*β_{y}/3^{1/2})*ΣK_{M}/(3*A*r_{A})

Find joint variables with Segment Lengths

S_{Calc}=[A*(Pr_{A}+Pr_{B}+Pr_{C})−F₀]/K_{F}L_{A}=3*A*Pr_{A}/K_{F}; Pr_{A}=K_{F}*L_{A}/(3*A) L_{B}=3*A*Pr_{B}/K_{F}; Pr_{B}=K_{F}*L_{B}/(3*A) L_{C}=3*A*Pr_{C}/K_{F}; Pr_{C}=K_{F}*L_{C}/(3*A) L_{OFFSET}=−F₀/K_{F}

S_{Calc}=[L_{A}+L_{B}+L_{C})/3+L_{OFFSET}

βₓ=(1/2)*A*r_{A}*(Pr_{B}+Pr_{C}−2*Pr_{A})/K_{M}

β_{y}=−(3^{1/2}/2)*A*r_{A}*(Pr_{B}−Pr_{C})/K_{M}L=r*β=r*[(3/2)*r*F/K_{M}]=(3/2)*r²*(A*Pr)/K_{M}L_{A}=(3/2)*r_{A}²*A*Pr_{A}/K_{M}; Pr_{A}=(2/3)*L_{A}*K_{M}/(r_{A}²*A) L_{B}=(3/2)*r_{A}²*A*Pr_{B}/K_{M}; Pr_{B}=(2/3)*L_{B}*K_{M}/(r_{A}²*A) L_{C}=(3/2)*r_{A}²*A*Pr_{C}/K_{M}; Pr_{C}=(2/3)*L_{C}*K_{M}/(r_{A}²*A)

βₓ=(1/3)*(L_{C}+L_{B}−2*L_{A})/r_{A}

β_{y}=(3^{1/2}/3)*(L_{C}−L_{B})/r_{A}

Joint Variables from Segment Lengths

S_{Calc}=[S_{A}+S_{B}+S_{C})/3

βₓ=(1/3)*(S_{C}+S_{B}−2*S_{A})/r_{A}

β_{y}=(3^{1/2}/3)*(S_{C}−S_{B})/r_{A}

Find Segment Lengths from Joint Variables.

β_{y}=(3^{1/2}/3)*(L_{C}−L_{B})/r_{A} L_{C}=L_{B}+(3/3^{1/2})*r_{A}*β_{y} (1)

βₓ=(1/3)*(L_{C}+L_{B}−2*L_{A})/r_{A}L_{C}=−L_{B}+2*L_{A}+3*r_{A}*βₓ (2)

Combine (1) and (2).

L_{B}+(3/3^{1/2})*r_{A}*β_{y}=−L_{B}+2*L_{A}+3*r_{A}*βₓ L_{A}=L_{B}−(3/2)*r_{A}*βₓ+(1/2)*(3/3^{1/2})*r_{A}*β_{y} (4)

S_{Calc}=[L_{A}+L_{B}+L_{C})/3+L_{OFFSET}L_{A}=−L_{B}−L_{C}+3*(S_{Calc}−L_{OFFSET})

Insert (1)

L_{A}=−L_{B}−{L_{B}+(3/3^{1/2})*r_{A}*β_{y}}+3*(S_{Calc}−L_{OFFSET}) L_{A}=−2*L_{B}—(3/3^{1/2})*r_{A}*β_{y}+3*(S_{Calc}−L_{OFFSET}) (5)

Combine (4) and (5)

L_{B}—(3/2)*r_{A}*βₓ+(1/2)*(3/3^{1/2})*r_{A}*β_{y}=−2*L_{B}−(3/3^{1/2})*r_{A}*β_{y}+3*(S_{Calc}−L_{OFFSET})

L_{B}+2*L_{B}=−(3/3^{1/2})*r_{A}*β_{y}−(1/2)*(3/3^{1/2})*r_{A}*β_{y}+(3/2)*r_{A}*βₓ+3*(S_{Calc}−L_{OFFSET})

3*L_{B}=−(3/2)*(3/3^{1/2})*r_{A}*β_{y}+(3/2)*r_{A}*βₓ+3*(S_{Calc}−L_{OFFSET})

L_{B}=S_{Calc}+(1/2)*r_{A}*βₓ−(1/2)*(3/3^{1/2})*r_{A}*β_{y}−L_{OFFSET} (6)

Insert (6) into (1)

L_{C}=(1/2)*r_{A}*βₓ−(1/2)*(3/3^{1/2})*r_{A}*β_{y}+S_{Calc}−L_{OFFSET}+(3/3^{1/2})*r_{A}*β_{y}

L_{C}=S_{Calc}+(1/2)*r_{A}*βₓ+(1/2)*(3/3^{1/2})*r_{A}*β_{y}−L_{OFFSET} (7)

Insert (6) into (4)

L_{A}=(1/2)*r_{A}*βₓ−(1/2)*(3/3^{1/2})*r_{A}*β_{y}+S_{Calc}−L_{OFFSET}−(3/2)*r_{A}*βₓ+(1/2)*(3/3^{1/2})*r_{A}*β_{y}

L_{A}=S_{Calc}−r_{A}*βₓ−L_{OFFSET} (8)

Segment Balloon Array Lengths from Joint Variables

L_{A}=S_{Calc}−r_{A}*βₓ−L_{OFFSET}

L_{B}=S_{Calc}+(1/2)*r_{A}*βₓ−(1/2)*(3/3^{1/2})*r_{A}*β_{y}−L_{OFFSET}

L_{C}=S_{Calc}+(1/2)*r_{A}*βₓ+(1/2)*(3/3^{1/2})*r_{A}*β_{y}−L_{OFFSET}

Segment Lengths from Joint Variables

S_{A}=S_{Calc}−r_{A}*βₓ

S_{B}=S_{Calc}+(1/2)*r_{A}*βₓ−(1/2)*(3/3^{1/2})*r_{A}*β_{y}

S_{C}=S_{Calc}+(1/2)*r_{A}*βₓ+(1/2)*(3/3^{1/2})*r_{A}*β_{y}

Find Segment Lengths from Lumen Pressures (Using Spring Pressure Math)

S=[S_{A}+S_{B}+S_{C})/3

S_{Calc}=[A*(Pr_{A}+Pr_{B}+Pr_{C})−F₀]/K_{F}S_{A}+S_{B}+S_{C}=3*[A*(Pr_{A}+Pr_{B}+Pr_{C})−F₀]/K_{F} (9)

βₓ=(1/3)*(S_{C}+S_{B}—2*S_{A})/r_{A}

βₓ=(1/2)*A*r_{A}*(Pr_{C}+Pr_{B}−2*Pr_{A})/K_{M} (1/3)*(S_{C}+S_{B}−2*S_{A})/r_{A}=(1/2)*A*r_{A}*(Pr_{C}+Pr_{B}−2*Pr_{A})/K_{M}S_{C}+S_{B}−2*S_{A}=(3/2)*A*r_{A}²*(Pr_{C}+Pr_{B}−2*Pr_{A})/K_{M} (10)

β_{y}=(3^{1/2}/3)*(S_{C}−S_{B})/r_{A}

β_{y}=(3^{1/2}/2)*A*r_{A}*(Pr_{C}−Pr_{B})/K_{M} (3^{1/2}/3)*(S_{C}−S_{B})/r_{A}=(3^{1/2}/2)*A*r_{A}*(Pr_{C}−Pr_{B})/K_{M}S_{C}−S_{B}=(3/2)*A*r_{A}²*(Pr_{C}−Pr_{B})/K_{M} (11)

Add (10) and (11)

2*S_{C}−2*S_{A}=(3/2)*A*r_{A}²*[(Pr_{C}+Pr_{B}−2*Pr_{A})+(Pr_{C}−Pr_{B})]/K_{M}

S_{C}−S_{A}=(3/2)*A*r_{A}²*(Pr_{C}−Pr_{A})/K_{M} (12)

Add (9) and (11)

S_{A}+2*S_{C}=3*[A*(Pr_{A}+Pr_{B}+Pr_{C})−F₀]/K_{F}+(3/2)*A*r_{A}²*(Pr_{C}−Pr_{B})/K_{M} (13)

Add (12) and (13)

3*S_{C}=3*[A*(Pr_{A}+Pr_{B}+Pr_{C})−F₀]/K_{F}+(3/2)*A*r_{A}²*(Pr_{C}−Pr_{B})/K_{M}+(3/2)*A*r_{A}²*(Pr_{C}−Pr_{A})/K_{M}

S_{C}=[A*(Pr_{A}+Pr_{B}+Pr_{C})−F₀]/K_{F}+(1/2)*A*r_{A}²*(2*Pr_{C}−Pr_{A}−Pr_{B})/K_{M} (14)

Substitute (14) into (11)

S_{B}=S_{C}−(3/2)*A*r_{A}²*(Pr_{C}−Pr_{B})/K_{M}

S_{B}=[A*(Pr_{A}+Pr_{B}+Pr_{C})−F₀]/K_{F}+(1/2)*A*r_{A}²*(2*Pr_{C}−Pr_{A}−Pr_{B})/K_{M}−(3/2)*A*r_{A}²*(Pr_{C}−Pr_{B})/K_{M}

S_{B}=[A*(Pr_{A}+Pr_{B}+Pr_{C})−F₀]/K_{F}+(1/2)*A*r_{A}²*(2*Pr_{C}−Pr_{A}−Pr_{B})−3*(Pr_{C}−Pr_{B})/K_{M}

S_{B}=[A*(Pr_{A}+Pr_{B}+Pr_{C})−F₀]/K_{F}+(1/2)*A*r_{A}²*(2*Pr_{B}−Pr_{C}−Pr_{A})/K_{M} (15)

Substitute (14) into (12)

S_{A}=S_{C}−(3/2)*A*r_{A}²*(Pr_{C}−Pr_{A})/K_{M}

S_{A}=[A*(Pr_{A}+Pr_{B}+Pr_{C})−F₀]/K_{F}+(1/2)*A*r_{A}²*(2*Pr_{C}−Pr_{B}−Pr_{A})/K_{M} −(3/2)*A*r_{A}²*(Pr_{C}−Pr_{A})/K_{M}

S_{A}=[A*(Pr_{A}+Pr_{B}+Pr_{C})−F₀]/K_{F}+(1/2)*A*r_{A}²*[(2*Pr_{C}−Pr_{B}−Pr_{A})−3*(Pr_{C}−Pr_{A})]/K_{M}

S_{A}=[A*(Pr_{A}+Pr_{B}+Pr_{C})−F₀]/K_{F}(1/2)*A*r_{A}^{2*}(2*Pr_{A}−Pr_{B}−Pr_{C})/K_{M} (16)

Segment Lengths from Lumen Pressure

S_{A}=[A*(Pr_{A}+Pr_{B}+Pr_{C})−F₀]/K_{F}+(1/2)*A*r_{A}²*(2*Pr_{A}−Pr_{B}−Pr_{C})/K_{M}

S_{B}=[A*(Pr_{A}+Pr_{B}+Pr_{C})−F₀]/K_{F}+(1/2)*A*r_{A}²*(2*Pr_{B}−Pr_{C}−Pr_{A})/K_{M}

S_{C}=[A*(Pr_{A}+Pr_{B}+Pr_{C})−F₀]/K_{F}+(1/2)*A*r_{A}²*(2*Pr_{C}−Pr_{A}−Pr_{B})/K_{M}

For segments with multiple lateral stiffness lengths, solve for a resultant bend constant by adding the contributions of the subsegments. The sub constants are scaled by the section's relative length to the whole segment. To avoid dividing by a zero length solve for the inverse of the bend constant.

dK_{M}′⁻¹=(S′/S)*(1/K_{M}′)

ΣK_{M}⁻¹=(1/S)*[(S′/K_{M}′)+(S″/K_{M}″)+ . . . (Sn′/K_{M}ⁿ′)]

ΣK_{M}=S/ [(S′/K_{M}′)+(S″/K_{M}″)+ . . . (Sn′/K_{M}ⁿ′)]

The Segment lengths for segments with multiple lateral stiffnesses can be solved as follows:

S_{A}=[A*(Pr_{A}+Pr_{B}+Pr_{C})−F₀]/K_{F}+(1/2)*A*r_{A}²*(2*Pr_{A}−Pr_{B}−Pr_{C})/ΣK_{M}

S_{B}=[A*(Pr_{A}+Pr_{B}+Pr_{C})−F₀]/K_{F}+(1/2)*A*r_{A}²*(2*Pr_{B}−Pr_{C}−Pr_{A})/ΣK_{M}

S_{C}=[A*(Pr_{A}+Pr_{B}+Pr_{C})−F₀]/K_{F}+(1/2)*A*r_{A}²*(2*Pr_{C}−Pr_{A}−Pr_{B})/ΣK_{M}

Find Lumen Pressures from Segment Lengths (Using Spring Pressure Math)

From (9), (10), and (11)

Pr_{A}+Pr_{B}+Pr_{C}=[(1/3)*K_{F}*(S_{A}+S_{B}+S_{C})+F₀]/A (17)

Pr_{C}+Pr_{B}−2*Pr_{A}=(2/3)*[K_{M}/(A*r_{A}²)]*(S_{C}+S_{B}−2*S_{A}) (18)

Pr_{C}−Pr_{B}=(2/3)*[K_{M}/(A*r_{A}²)]*(S_{C}−S_{B}) (19)

Add (18) and (19)

(Pr_{C}+Pr_{B}−2*Pr_{A})+(Pr_{C}−Pr_{B})=(2/3)*[K_{M}/(A*r_{A}²)]*(S_{C}+S_{B}−2*S_{A})+(2/3)*[K_{M}/(A*r_{A}²)]*(S_{C}−S_{B})

Pr_{C}−Pr_{A}=(2/3)*[K_{M}/(A*r_{A}²)]*(S_{C}−S_{A}) (20)

Subtract (17) from (18)

(Pr_{C}+Pr_{B}−2*Pr_{A})−(Pr_{A}+Pr_{B}+Pr_{C})=(2/3)*[K_{M}/(A*r_{A}²)]*(S_{C}+S_{B}−2*S_{A})−[(1/3)*K_{F}*(S_{A}+S_{B}+S_{C})+F₀]/A

−3*Pr_{A}=(2/3)*[K_{M}/(A*r_{A}²)]*(S_{C}+S_{B}−2*S_{A})−[(1/3)*K_{F}*(S_{A}+S_{B}+S_{C})+F₀]/A

Pr_{A}=(1/3)*[(1/3)*K_{F}*(S_{A}+S_{B}+S_{C})+F₀]/A+(1/3)*(2/3)*[K_{M}/(A*r_{A}²)]*(2*S_{A}−S_{C}−S_{B}) (21)

Substitute (21) into (20)

Pr_{C}=Pr_{A}+(2/3)*[K_{M}/(A*r_{A}²)]*(S_{C}−S_{A})

Pr_{C}=(1/3)*[(1/3)*K_{F}*(S_{A}+S_{B}+S_{C})+F₀]/A+(1/3)*(2/3)*[K_{M}/(A*r_{A}²)]*(2*S_{A}−S_{C}−S_{B})+(2/3)*[K_{M}/(A*r_{A}²)]*(S_{C}−S_{A})

Pr_{C}=(1/3)*[(1/3)*K_{F}*(S_{A}+S_{B}+S_{C})+F₀]/A+(1/3)*(2/3)*[K_{M}/(A*r_{A}²)]*(2*S_{A}−S_{C}−S_{B})+(1/3)*(2/3)*[K_{M}/(A*r_{A}²)]*(3*S_{C}−3*S_{A})

Pr_{C}=(1/3)*[(1/3)*K_{F}*(S_{A}+S_{B}+S_{C})+F₀]/A+(1/3)*(2/3)*[K_{M}/(A*r_{A}²)]*(2*S_{C}−S_{A}−S_{B}) (22)

Substitute (22) into (19)

Pr_{B}=Pr_{C}−(2/3)*[K_{M}/(A*r_{A}²)]*(S_{C}−S_{B})

Pr_{B}=(1/3)*[(1/3)*K_{F}*(S_{A}+S_{B}+S_{C})+F₀]/A+(1/3)*(2/3)*[K_{M}/(A*r_{A}²)]*(2*S_{C}−S_{A}−S_{B})−(2/3)*[K_{M}/(A*r_{A}²)]*(S_{C}−S_{B})

Pr_{B}=(1/3)*[(1/3)*K_{F}*(S_{A}+S_{B}+S_{C})+F₀]/A+(1/3)*(2/3)*[K_{M}/(A*r_{A}²)]*(2*S_{C}−S_{A}−S_{B}) (1/3)*(2/3)*[K_{M}/(A*r_{A}²)]*(3*S_{C}−3*S_{B})

Pr_{B}=(1/3)*[(1/3)*K_{F}*(S_{A}+S_{B}+S_{C})+F₀]/A+(1/3)*(2/3)*[K_{M}/(A*r_{A}²)]*(2*S_{B}−S_{C}−S_{A}) (23)

Lumen Pressures from Segment Lengths (Using Spring Pressure Math)

Pr_{A}=(1/3)*[(1/3)*K_{F}*(S_{A}+S_{B}+S_{C})+F₀]/A+(1/3)*(2/3)*[K_{M}/(A*r_{A}²)]*(2*S_{A}−S_{B}−S_{C})

Pr_{B}=(1/3)*[(1/3)*K_{F}*(S_{A}+S_{B}+S_{C})+F₀]/A+(1/3)*(2/3)*[K_{M}/(A*r_{A}²)]*(2*S_{B}−S_{C}−S_{A})

Pr_{C}=(1/3)*[(1/3)*K_{F}*(S_{A}+S_{B}+S_{C})+F₀]/A+(1/3)*(2/3)*[K_{M}/(A*r_{A}²)]*(2*S_{C}−S_{A}−S_{B})

For segments with multiple lateral stiffness lengths, solve for a resultant bend constant by adding the contributions of the subsegments. The sub constants are scaled by the section's relative length to the whole segment. To avoid dividing by a zero length solve for the inverse of the bend constant.

dK_{M}′⁻¹=(S′/S)*(1/K_{M}′)

ΣK_{M}⁻¹=(1/S)*[(S′/K_{M}′)+(S″/K_{M}″)+ . . . (Sn′/K_{M}ⁿ′)]

ΣK_{M}=S/ [(S′/K_{M}′)+(S″/K_{M}″)+ . . . (Sn′/K_{M}ⁿ′)]

The Segment lengths for segments with multiple lateral stiffnesses can be solved as follows:

Pr_{A}=(1/3)*[(1/3)*K_{F}*(S_{A}+S_{B}+S_{C})+F₀]/A+(1/3)*(2/3)*[ΣK_{M}/(A*r_{A}²)]*(2*S_{A}−S_{B}−S_{C})

Pr_{B}=(1/3)*[(1/3)*K_{F}*(S_{A}+S_{B}+S_{C})+F₀]/A+(1/3)*(2/3)*[ΣK_{M}/(A*r_{A}²)]*(2*S_{B}−S_{C}−S_{A})

Pr_{C}=(1/3)*[(1/3)*K_{F}*(S_{A}+S_{B}+S_{C})+F₀]/A+(1/3)*(2/3)*[ΣK_{M}/(A*r_{A}²)]*(2*S_{C}−S_{A}−S_{B})

Check for Accuracy and Obtainability

Setup Inverse Jacobian to solve for Segment Balloon Array lengths and repeat until joint (j) variable errors meet minimum condition. Check that α, β, and S meet viable solutions.

j_{calc}−j_{desired}<jₑᵣᵣₒᵣ

|α_{calc}|<360°

β_{Calc}>β_{Min} (some amount greater than Zero),

β_{Calc}<β_{Max} (may be 180° or 360°)

S_{Calc}>S_{Min} &<S_{Max}

Solve for segment force and moment

F=K_{F}*Sᵢ+F₀

F=A*(Pr_{A}+Pr_{B+Pr}_{C})

M=K_{M}*β+M₀

M=A*r_{A}*(pr_{A}²+Pr_{B}²+Pr_{C}²−Pr_{A}*Pr_{B}−Pr_{B}*Pr_{C}−Pr_{C}*Pr_{A}))^{1/2}

Solve for Segment Length and Angle

Sᵢ=(F−F₀)/K_{F} Check for equivalency

β=(M−M₀)/K_{M} Check for equivalency

Communications Between Robot Articulation Controller Module and Simulation/Display Processor Module

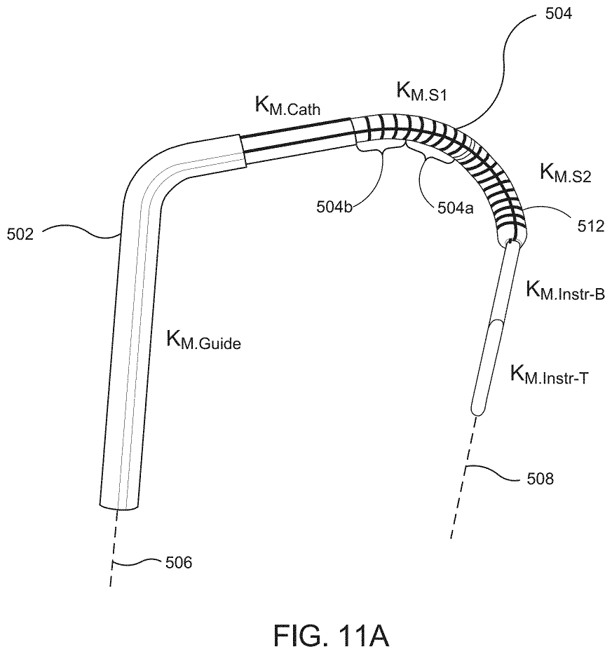

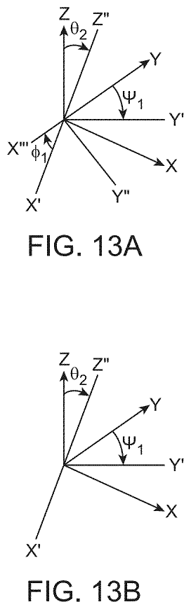

Referring now to FIGS. 9 and 12, the module used to control movement of the actual catheter in the surgical workspace may have a 3-D workspace with a first reference frame (sometimes referred to herein as the ROBOT reference frame), while the simulation module used to calculate virtual movement of a virtual catheter in a virtual workspace may use a second reference frame (such as a simulation reference frame, sometimes referred to herein as the UNITY™ reference frame) that is different from the first reference frame. For example, the ROBOT reference frame may be a right-hand-rule reference frame with the positive Z orientation being vertically upward, while the virtual or UNITY™ reference frame may be a left-hand-rule reference frame with the positive Y axis being upward. The calculations performed in these different environments may also have differences, such as relying predominantly or entirely on Euler angles and transformation matrices in the ROBOT calculations and relying more predominantly or even entirely on quaternions and related vector manipulations in the virtual reference frame, with any Euler angle-related transformations optionally based on a different set of rotation axes, different rotation orientations, and/or different order of rotations. Nonetheless, there may be advantages to sending data in both directions (from the virtual environment to the actual robot, and from the actual robot to the virtual environment) during a procedure so as to provide enhanced correlation between the virtual and actual poses and system performance. Rotations between these different reference frames are illustrated in FIGS. 12, 13A, and 13B

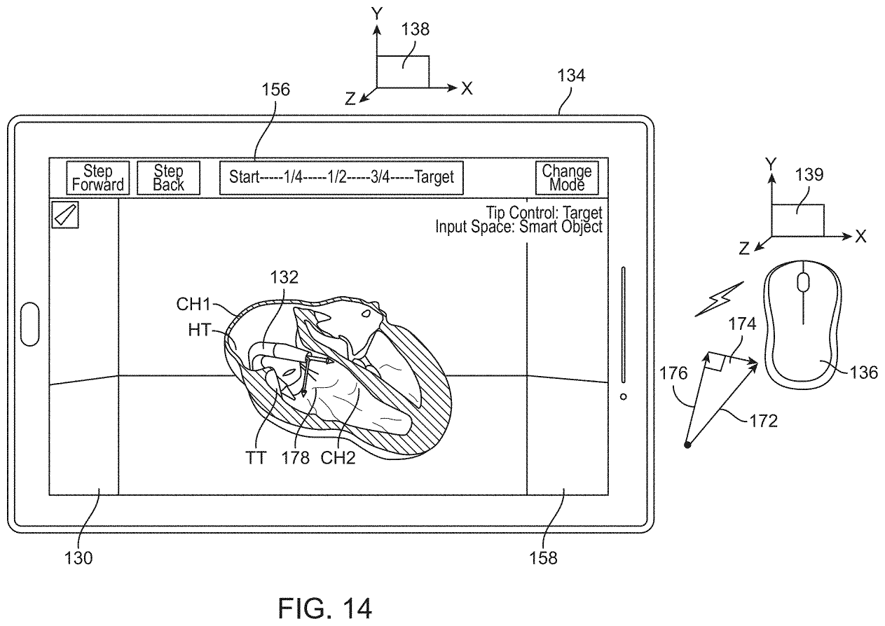

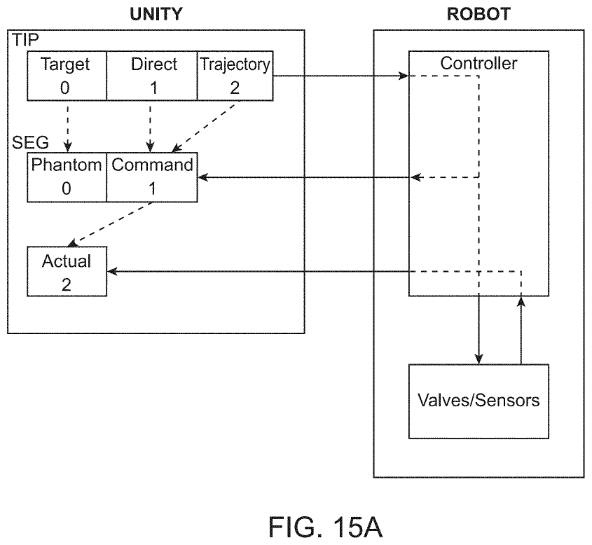

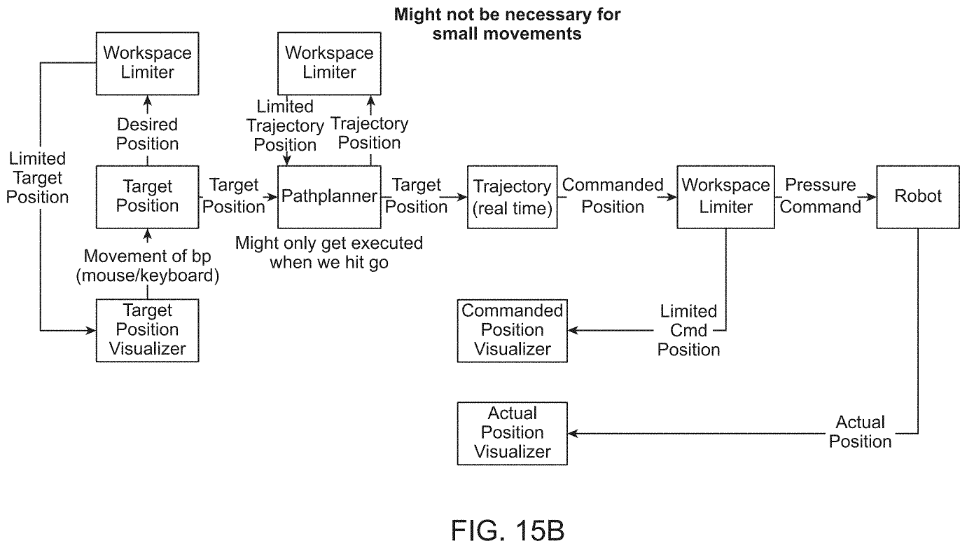

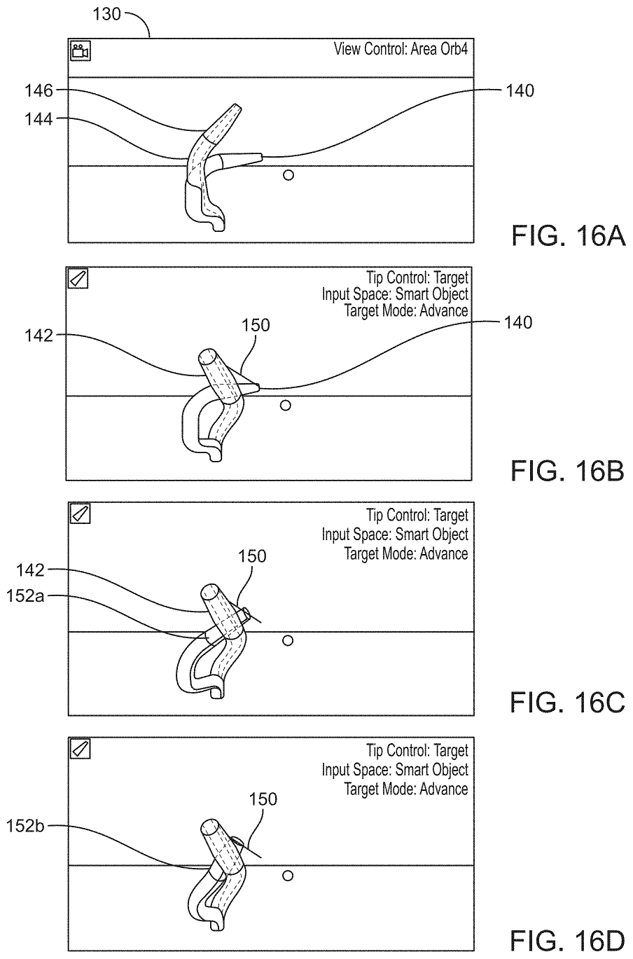

Referring now to FIGS. 14 and 16A-16D, a method for using a computer controlled flexible catheter system for aligning a virtual and/or actual therapeutic or diagnostic tool with a target tissue can be understood. As seen in a display 130, an image of heart tissue adjacent an internal site (here including one or more chamber of the heart CH1, CH2) in a patient is being shown, with the heart tissue image typically being acquired prior to or during a procedure, or simply being based on a model. Target tissue TT may comprise a tissue structure of the tricuspid or mitral valve, a septum or other wall bordering a chamber of the heart, an os of a body lumen, or the like. An image of a catheter having an elongate body 132 has been inserted into the patient or superimposed on the tissue image, the elongate body having a receptacle to support the tool. The position and orientation of the receptacle define a first pose of the tool within the 3-D internal surgical site. Display 130 may comprise a surgical monitor, a standard desktop computer monitor, or a display of a notebook computer, with the exemplary display in this embodiment comprising a touchscreen of a tablet 134. The touchscreen may be used for both input into and output from the data processing system. An at least 2-D input device 136 separate from the touchscreen may optionally be used for input with or instead of the touchscreen, and a display housed in a structure separate from the tablet may be coupled to a processor of the tablet for use with or instead of display 130.

Referring still to FIG. 14, display 130 generally has an image display plane 138 with a first or lateral orientation X and a second or transverse orientation Y. A third or Z orientation may extend into display 130 (away from the user) in a left-hand or Unity™ display coordinate system. Input commands on a touchscreen display 130 will typically comprise position changes with components in the X and Y orientations. Similarly, input commands sensed by input device 136 may have X and Y component orientations. Z components of the input commands may be sensed as pinch gestures on a touchscreen, rotation of a scroll wheel of a mouse, axial movement of a 3-D input device, or the like. Note that the display coordinate system or reference frame may be referred to as the camera reference frame, and may be offset from the world coordinate system of the virtual and/or real 3D workspace. An image plane may be parallel to the display plane adjacent the tissue at a selectable distance from the display plane, or may be manipulatable by the user so as to set an offset angle between the display and image planes.

Referring now to FIGS. 16A-16C, it may be beneficial to allow the user to move a virtual receptacle around within the internal worksite to more than one candidate pose to evaluate a number of potential poses and pick a particularly desired or target pose, but without having the delay or to impose the potential trauma of actually moving the catheter to or through poses that are not ideal. Toward that end, the processor may include a module configured for receiving, from a user, input for moving the receptacle and tool of the catheter from a first pose 140 to a second pose 142 within the internal surgical site. The input may make use of a virtual receptacle, tool, and/or catheter 144 having an image that moves in display 130, with the virtual receptacle often defining one or more candidate or intermediate input poses 146 after moving from the first pose 140 and before the being moved to the desired second pose 142. Rather than treating the phantom catheter as a master-slave input during such a movement, the processor of the system may drive the actuators coupled to the catheter so that the receptacle remains fixed at the first location while the user inputs commands on the touchscreen or via another input device to identify the desired receptacle pose. Once the desired pose has been established, the processor can receive a movement command to move the receptacle. In response, the processor may transmit drive signals to a plurality of actuators so as to advance the receptacle along a trajectory 150 from the first pose toward the second pose. Advantageously, the trajectory can be independent of the intermediate, non-selected candidate input pose 146, as well as from the (potentially meandering) trajectory input by the user to and from any number of intermediate poses 146.

As can be understood with reference to FIGS. 16B-16D, when the receptacle of the catheter is in the first pose 140 and the user inputs the second pose 142 into the processor (preferably using a virtual catheter image moving in the 3D workspace), trajectory 150 from the first pose to the second pose will often comprise a relatively complex series of actuator drive signals that move the catheter body that drive the actuators in sequence to induce movement in a plurality of degrees of freedom that result in the desired change in both position and orientation of the therapeutic tool. Advantageously a quaterion-based trajectory planning module (optionally included in the input processor) may calculate the trajectory as a linear interpolation between the first and second poses, and the user may optionally manipulate the trajectory as desired to avoid deleterious tissue engagement or the like. Regardless, it will often be desirable for the user to maintain close control over advancement of the catheter along the trajectory. Toward that end, the processor may receive a movement command from the user to move along an incomplete spatial portion of trajectory 150 from the first pose 140 to the second pose 142, and to stop at an intermediate pose 152a between the first pose and the second pose. For example, the movement command may be to move a rational fraction of the trajectory, such as ¼ of the trajectory, ⅛ of the trajectory, or the like. The user may gradually or incrementally complete the trajectory in one or more portions 152a, 152b, . . , stop after one or more spatial portions and choose a new desired target pose, or even move back along the trajectory one or more portions away from the second pose and back toward the first pose. The movement command may be entered as a series of steps (such as using forward and backward step buttons 154a, 154b of the touchscreen in FIG. 14, forward and backward arrow keys on a keyboard, or the like) or using a single continuous linear input sensor (such as a scroll wheel of mouse or linear scale 156 of a touchscreen as in FIG. 14). In response to the movement command, the processor can transmit drive signals to a plurality of actuators coupled with the elongate body so as to move the receptacle toward the intermediate pose, optionally using the motion control arrangement described above.

Referring now to FIGS. 14 and 17A-18, exemplary methods for using a planar input device (such as a touchscreen 158 of tablet 134 or a mouse input device 136) to achieve accurately controlled motion in up to three positional degrees of freedom and one, two, or three orientational degrees of freedom can be understood. More specifically, these methods and systems can be used for manipulating a real and/or virtual elongate tool 162 in a three-dimensional workspace 164. The tool has an axis 166, and may be supported by a flexible catheter or other support structure extending distally along the axis to the tool, as described above. As can also be understood from the description above, an input/output (I/O) system 111 (see FIG. 5) can be configured for showing an image of the tool in a display 168 and for receiving a two-dimensional input from a user. The I/O system will often have at least one plane (see image plane 138, and input plane 139 of FIG. 14) and the axis of the tool as shown in the tool image may have a display slope 170 along the display plane. The user may enter a movement command by moving a mouse on the input plane, by dragging a finger along touchscreen 158, or the like, thereby defining an input 172 along the input plane. A first component 174 of the input can be defined along a first axis corresponding to the tool display slope, and a second component of the input 176 can be defined along a second axis on the input plane perpendicular to the tool display slope.

To facilitate precise control over both the position and orientation of the tool in the workspace, the processor of the system may have a translation input mode and an orientation input mode. When the processor is in the orientation mode, the first component 174 of the input 172 (the portion extending along the axis 166 of the tool as shown in the image), will typically induce rotation of the tool in three-dimensional workspace 158 about a first rotational axis 178 that is parallel to the display plane 138 and perpendicular to the tool axis 166. In response to the second component 174 of the input, the processor can induce rotation of the tool and tool image about a second rotational axis 180 that is perpendicular to the tool axis 166 and also to the first rotational axis 178. Using vector notation, the first rotational axis VN can be calculated from the first and second components of the input V₁, V₂ as:

$$\frac{{V}_{1} \times {V}_{2}}{\vert V 1 * V 2 \vert} = {V}_{N}$$

The second rotational axis V_{S} can then be calculated from the inverse of the first rotational axis V_{NS} and from the axis 166 of the tool V_{T} as follows:

V_{NS}×V_{T}=VS

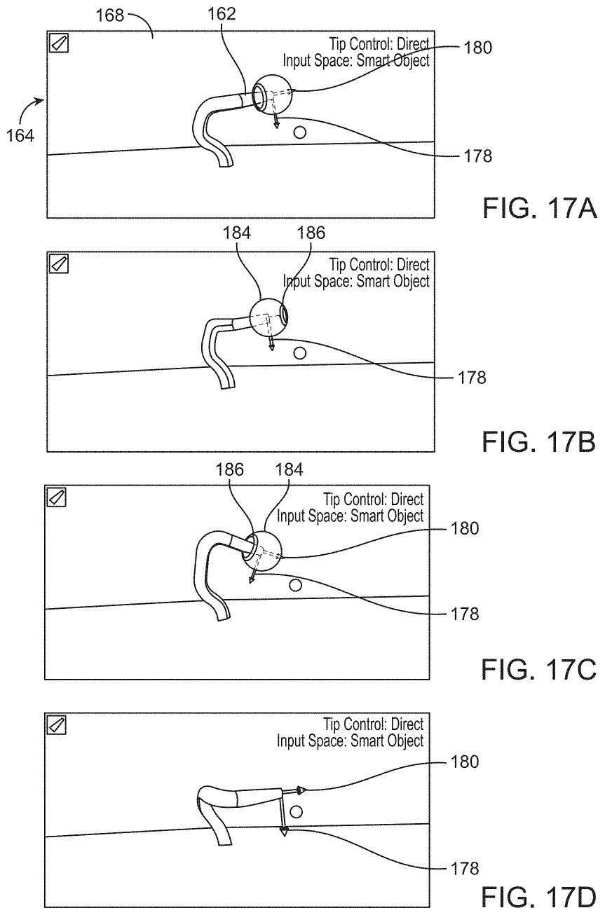

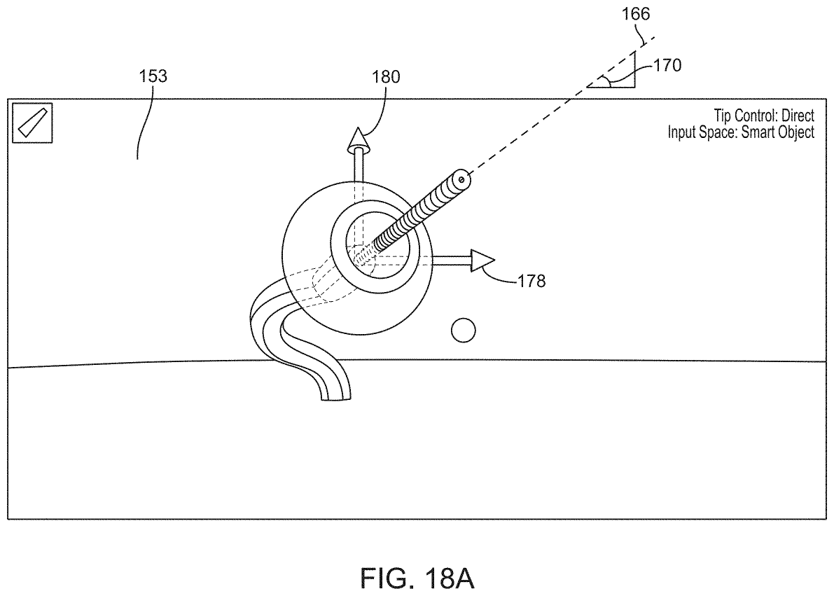

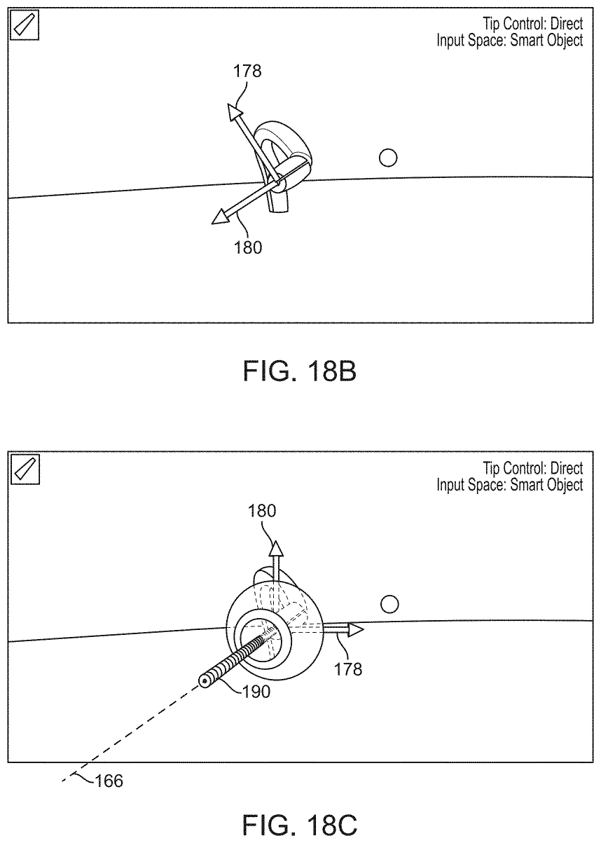

The tool axis 166 and first and second rotational axes 178, 180 will typically intersect at a spherical center of rotation at a desired location along the tool, such as at a proximal end, distal end, or mid-point of the tool. To help make the rotational movement intuitive, the processor can superimpose an image of a spherical rotation indicator such as a transparent ball 184 concentric with the center of rotation. Superimposing rotation indicia such as a concentric ring 186 encircling the tool axis 166 on the side of ball 184 oriented toward the user can further help make the orientation of rotation predictable, as input movement of the mouse or movement of the user's finger on a touchscreen in an input direction along the input plane can move the rotation indicia in the same general direction as the input movement, giving the user the impression that the input is rotating the ball about the center of rotation with the input device. The rotation indicia will preferably stay at a fixed relationship relative to the center of rotation and tool axis during a rotation of the tool, but may switch sides of the ball when a rotation increment is complete (as can be understood by comparing FIGS. 17B and 17C. As can be understood with reference to FIGS. 18A-C, when the tool axis 166 is within a desired angle range of normal to the display plane, the rotational axes 178, 180 may revert to extending along lateral and transverse display orientations (see X-Y orientations along plane 138 in FIG. 14).

When a planar input device is in use and the input processor is in an object-based translation mode, input movement along the axis slope 170 may result in translation of tool 162 in workspace 164 along the second rotational axis 180. Input command movement along the display and/or image plane perpendicular to the axis slope 170 may induce translation of tool parallel to the first rotational axis 178. Advantageous movement of the tool along the axis of the catheter when using a mouse or the like can be induced by rotating a scroll wheel. In a view-based translation mode, input command movement along the X-Y input plane 139 can induce corresponding movement of the tool along the X-Y display plane 138. Scroll wheel rotation in the view-based translation mode can induce movement into and out of the display plane (along the Z axis identified adjacent display plane 138 in FIG. 14). Selection between input modes may be performed by pushing different buttons of a mouse during input, using menus, and/or the like. Optionally, a ruler 190 having axial measurement indicia may be superimposed by the processor extending distally from the tool along axis 166 to facilitate axial measurement of tissues, alignment with and proximity of the tool to the target tissues, and the like. Similarly, lateral offset indicia such as a series of concentric measurement rings of differing radii encircling axis 166 at the center of rotation can help measure a lateral offset between the tool and tissues, the size of tissue structures, and the like.

A number of additional input and/or articulation modes may be provided. For example, the user may select a constrained motion mode, in which movement of the tool or receptacle is constrained to motion along a plane. The plane may be parallel to the display plane and the processor may maintain a separation distance between the tool and the constraint plane (which may be coincident with or near an imaging plane of the imaging system) when the planar movement mode is initiated. This can help keep the tool in view of, for example, a planar ultrasound imaging system, while facilitating movement of the tool relative to tissue structures while both remain at good imaging depths. Alternatively, the user may use the input system to position a constraint plane or other surface at a desired angle and location within the 3-D workspace. Alternative constraint surfaces may allow movement on one side of the surface and inhibit motion beyond the surface, or the like. Such constrained motion can be provided by constraining the above catheter motion arrangements with the equation for the surface, such as with the equation for a plane (aX+bY+cZ+d=0). Hence, a wide variety of alternative surface-based or volume-based movement constraints may be provided.

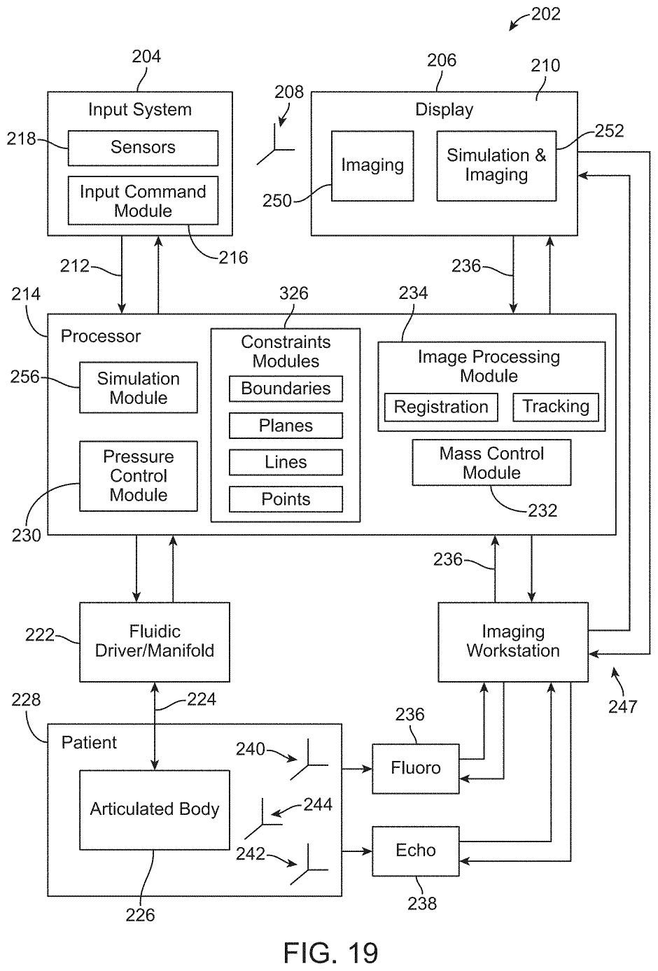

Referring now to FIG. 19, a functional block diagram schematically illustrates aspects of an exemplary data architecture of a fluid-driven structural heart therapy delivery system 202. In general terms a user inputs user commands into an input system 204 with reference to images presented by a display 206, with the input commands typically being entered in an input and display reference frame 208 which may optionally be defined in part by a plane 210 of display 206, and in part by 3D orientations of the objects and tissues represented by the displayed images. Those 3D orientations are, in turn, determined by an orientation and position of an image capture device (used to acquire the image) relative to the objects, or in the case of virtual or model objects, by the calculated camera frame. Hence, display frame 208 is sometimes referred to herein as a camera reference frame.

Referring still to FIG. 19, input commands 212 are transmitted from input system 204 to a processor 214, which may optionally transmit robotic system state data or the like back to the input system. Processor 214 includes data processing hardware, software, and/or firmware configured to perform the methods described herein, with the functional combinations of these components which work together to provide a particular data processing functionality often being described herein as a module. As noted above, the distribution of these data processing modules need not (and often will not) correspond exactly to separation of physical components of the fluid-driven robotic system. For example, an input command module 216 that generates input commands 212 in response to signals from movement sensors 218 may include software components running on a processor board of a 6 DOF input device, and may also include other software components running on a board of a desktop or notebook computer. Processor 214 sends actuator commands 220 in the form of valve commands to a fluidic driver/manifold 222 resulting in the transmission of inflation fluid 224 to the articulation balloon array of an elongate articulated body 226, thereby inducing movement of the tool receptacle and tool in the patient body 228. The pressures of the balloon inflation lumens provides a feedback signal that can be used by a pressure control module 230 of processor 214 to help determine an inflation state of the balloon array and an articulation state of the catheter or other elongate body.

As can be understood with reference to FIG. 19, along with pressure signals from the articulation balloon array, additional feedback may be employed by the data processing components of delivery system 202 to generate actuator commands. For example, a mass control module 232 of processor 214 may track an estimated mass in the subsets of balloons in the balloon array whenever the valves of the manifold open to add inflation fluid to or release inflation fluid from a balloon inflation channel, so that the absolute articulation state of the articulated body can be estimated from the inflation fluid mass and the sensed pressure of the lumen. Articulation state may also be sensed by an optical fiber sensing system having an optical fiber sensor extending along the elongate body. Still further articulation feedback signals may be generated using an image processing module 234 based on image signals 236 generated using a fluoroscopy image capture device 236, an ultrasound image capture device 238, an optical image capture device, and MRI system, and/or the like. Preferably the image processing module 234 includes a registration module that determines transformations that can register fluoro image data obtained by the fluoro system 236 in a fluoro reference frame 240 with echo image data obtained by echo system 238 in an echo reference from 242 so as to provide feedback on catheter movement in a unified catheter reference frame 244. Such registration of the fluoro and echo data may make use of known and/or commercially available data fusion technologies, including those commercialized by Philips, Siemens, and/or General Electric, and/or registration of the catheter position and orientation may make use of known catheter voxel segmentation such as those described by Yang et al. in an article entitled “Catheter segmentation in three-dimensional ultrasound images by feature fusion and model fitting,” Journal of Medical Imaging 6(1), 015001 (January-March 2019), or as the processor 214 of system 202 will often have access to a-priori knowledge of the catheter structure and current knowledge of its bend state, to any of the earlier known segmentation approaches. As such registration may take more than a second (typically taking more than a 10 seconds, or even more than a minute), tracking ongoing movements of the registered catheter from streaming image data intermittently or in real time may employ a faster tracking technique, such as that described by Nadeau et al. in the article entitled “Target Tracking in 3D Ultrasound Volumes by Direct Visual Serving,” The Hamlyn Symposium on Medical Robotics (2012).

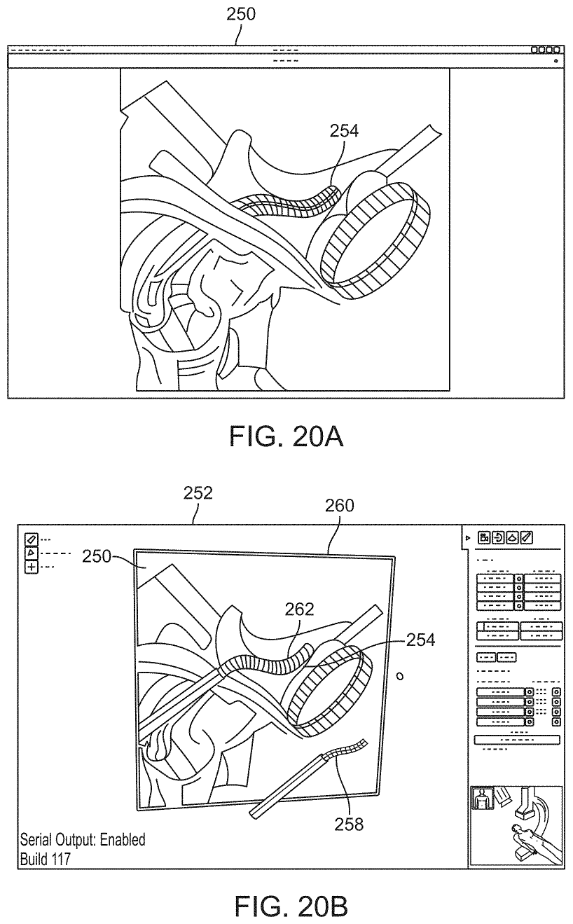

Referring now to FIGS. 19, 20A, and 20B, display 206 will often present acquired images 250 and auxiliary images 252, the auxiliary images optionally comprising virtual images showing a simulation or graphical model of the articulated body (as discussed above) and/or an augmented image including a simulation of the articulated body superimposed on an acquired image. As seen in the simulated intracardiac fluoroscopic acquired image 250 of FIG. 20A (actually an optical image taken in a benchtop model), the image data acquired by the optical imaging system is used to present a real-time 2D image of articulated body 254 and surrounding tissues. This image data is combined with 3D model data generated by a simulation module 256 of processor 214 so that auxiliary image 252 includes one, two, or all of: i) a 3D image of the model articulated elongate body 258, ii) at least a portion of the acquired real-time 2D image 250 on an associated image plane 260 in the display space 208, and iii) a 2D projection 262 of the model onto the 2D image plane superimposed on the acquired 2D image. The superimposed 2D model or virtual image 262 and acquired image of the articulated body 254 can help the user or an automated image processing system to register, track, and verify alignment of the model with the actual catheter, so that the augmented reality display 252, often with acquired display 250, allows enhanced image guidance when aligning a tool supported by the catheter with a target tissue.

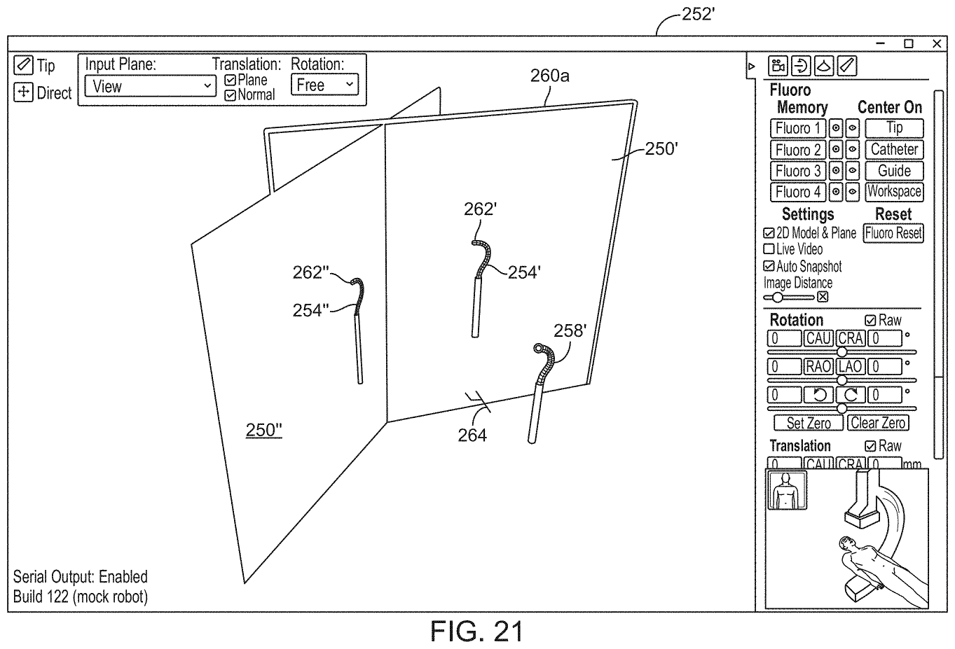

Referring now to FIGS. 19, 20B, and 21, auxiliary image 252 will often comprise a hybrid 2D/3D image presenting a 2D image elements included within a 3D image environment. More specifically, as noted above some or all of acquired image 250, 250′ is included in auxiliary image 252, 252′, with the acquired image data being manipulated so as to appear on an image plane 260, 260a at a desired angle and location within the display space 208. A 3D virtual image of a model of the catheter or other elongate body 258, 258′ is also presented, and while the acquired image remains a 2D image, it can be scaled, tilted, etc. and/or the 3D model can be oriented (and optionally scaled) so that an orientation of the 2D acquired image of the catheter corresponds to a projection of the 3D model onto associated image plane 260, 260a, preferably along a normal to the image plane 264. As noted above, a 2D projection of the 3D virtual image onto the image plane may also be included. As can be understood with reference to FIG. 21, a second acquired image 250″ at a different orientation than the first acquired image 250 may be presented on a second image plane 260b, typically including another 2D image of the catheter 254″ and/or a second 2D projection 262″ of the 3D virtual catheter model 258′ onto the image plane. Note that one or both of the capture images may be generated from recorded image data (optionally being a recorded still image or a recorded video clip, such as from a single plane fluoro system with the C-arm at a prior offset angle) so that the recorded image of the catheter may not move in real time, but can still provide beneficial image guidance for alignment of a virtual catheter relative tissues.

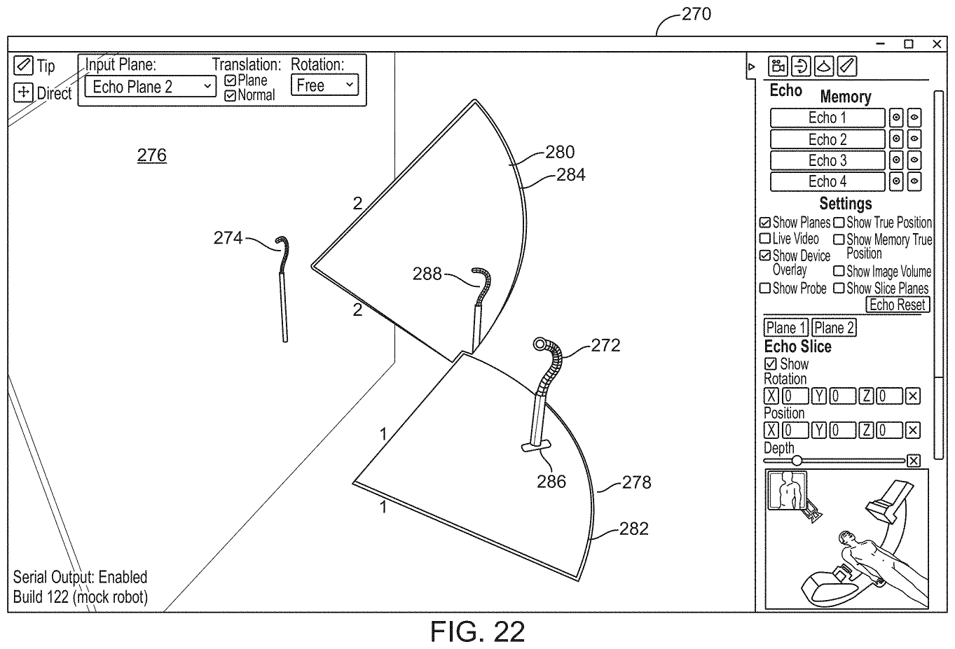

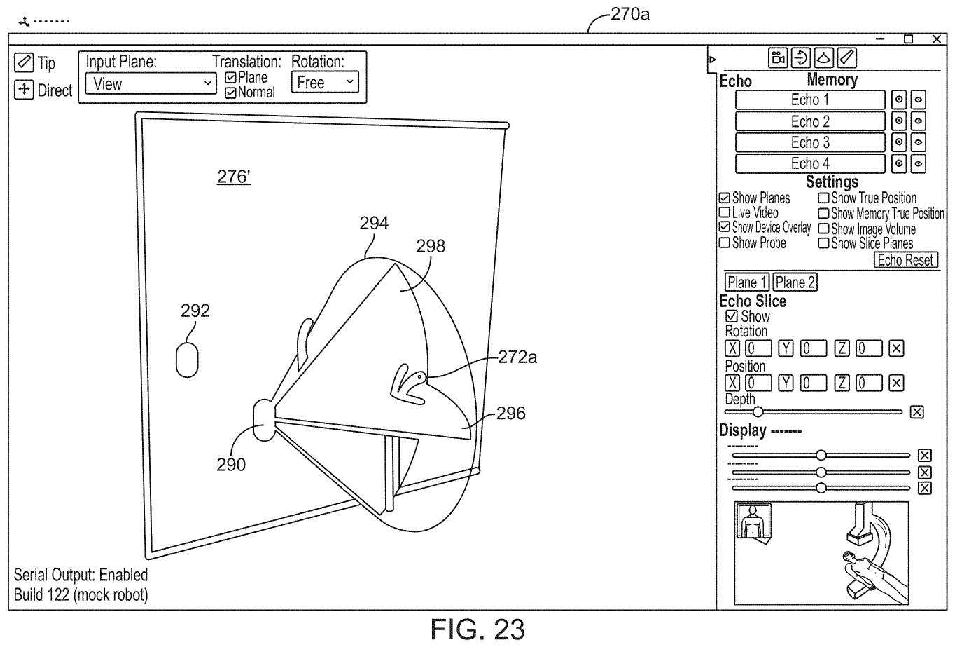

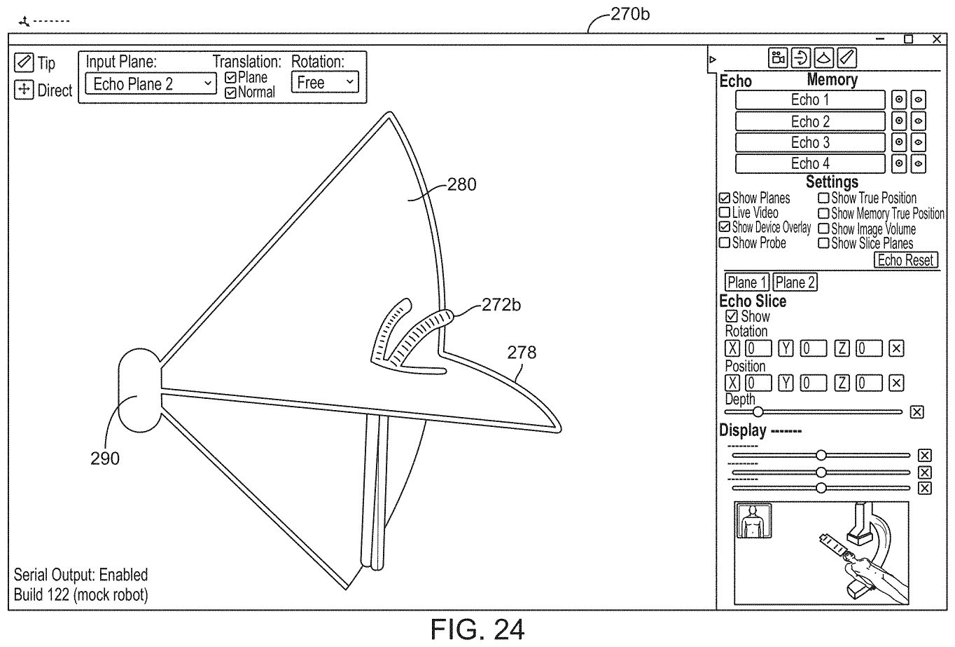

Referring now to FIGS. 22-25, alternative hybrid 2D/3D transesophageal echocardiography (TEE) or intracardiac echocardiography (ICE) images 270, 270a, 270b, 270c includes many elements related to those described above, including a 3D virtual model image and an associated 2D virtual model superimposed projection 274 onto an image plane of an acquired fluoroscopic image 276. For example, as seen in FIG. 22, hybrid TEE or ICE image 270 also includes first and second acquired 2D echo images 278, 280 on associated echo planes 282, 284, with projected 2D virtual model images 286 288 superimposed on the acquired echo images along normals to the planes. As schematically shown in FIGS. 23 and 24, echo images 278, 280 are acquired using a TEE or ICE probe 290, and an image 292 of TEE or ICE probe 290 will often be seen in the acquired fluoroscopic image 276′. This facilitates the use of known data fusion techniques for registering the image data from the fluoro system with the image data from the echo system. TEE or ICE probe 290 will optionally comprise a volumetric TEE or ICE probe acquiring image data throughout a volume 294. Optionally, 3D acquired images of the catheter can be presented in hybrid TEE image 270 using such volumetric TEE capabilities. Nonetheless, image guidance for alignment of the catheter-supported tool may benefit from presenting acquired echo image slices obtained from intersecting echo image planes 296, 298 within the echo data acquisition volume 294. To facilitate clear viewing of the acquired 2D echo images (often augmented with 2D virtual catheter images superimposed thereon) the echo image display planes 282, 284 can be offset from the 3D catheter virtual model 272, 272c, optionally along normals of the associated image planes.

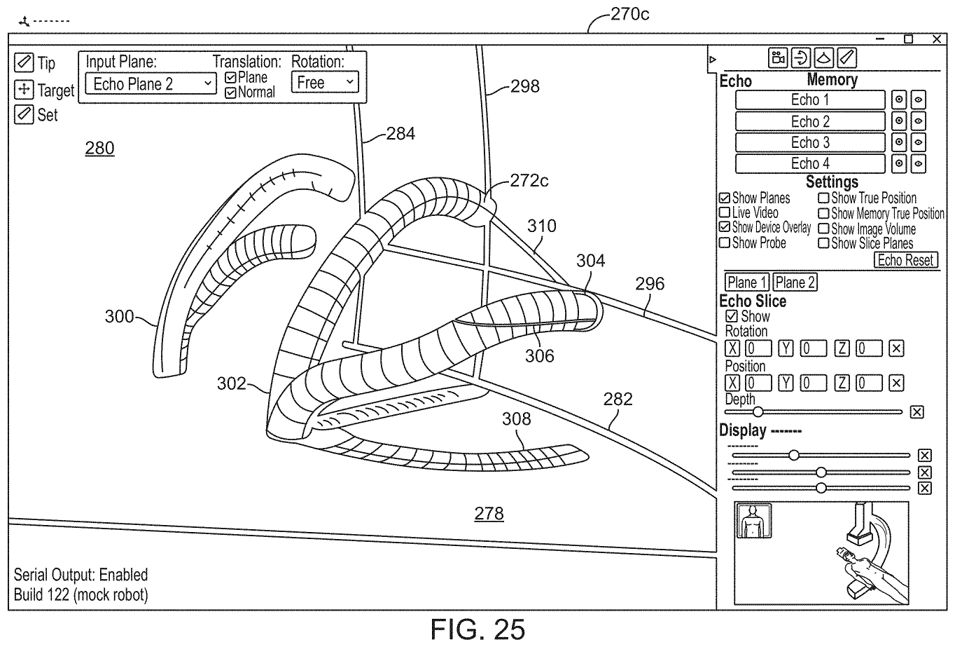

Referring now to FIG. 25, hybrid images may advantageously present combinations of acquired images of an actual catheter 300, images of a virtual or model catheter 302, images of phantom or candidate catheter poses 304, or all three. The acquired images will often be presented as 2D images on associated image planes; the phantom and virtual or model catheter images optionally being presented as 3D model images or 2D images superimposed on the acquired images or both. When 3D model image (for example, 3D phantom image 304) will be presented together with ultrasound image slices (such as along first echo plane 296) it can be beneficial to highlight a model/slice intersection 306 of the 3D model, and to superimpose a 2D intersection image 308 of the highlighted intersection on the acquired echo image plane 278. As can be understood with reference to FIGS. 16A-16D, 19, 25, and the associated text, phantom or candidate pose virtual models of the catheters or other articulated elongate bodies can be based initially on a calculated pose of the catheter that is virtually modified by entering movement commands using an input system 204. The modified pose can be determined using a simulation module 256 of processor 214. The system can display trajectory 310 between the model pose and the candidate pose in 3D with the 3D models and the user can input commands to the input system to advance along the trajectory, stop, or retreat along the trajectory as described above.

Referring to FIGS. 19 and 22-25, in the lower right portion of the hybrid display is a graphical reference frame indicator showing offset orientations of the display frame and at least one imaging system frame. The reference frame indicator optionally presents overall tissue position and an associated reference frame 246 graphically, preferably by including an image of some or all of the patient body (such as the body, torso, heart, or head) indicative of a position and orientation of tissues targeted for treatment or diagnosis. An orientation of an image capture device and associated reference frame 240, 242 relative to the patient body is also presented graphically with an image capture orientation indicator, preferably using an image of the associated imaging device (such as an image of a C-arm, TEE probe, ICE probe, or the like). An orientation of the display reference frame 208 relative to the patient body is presented graphically using, for example, a schematic image of a camera, a reference frame, or the like.

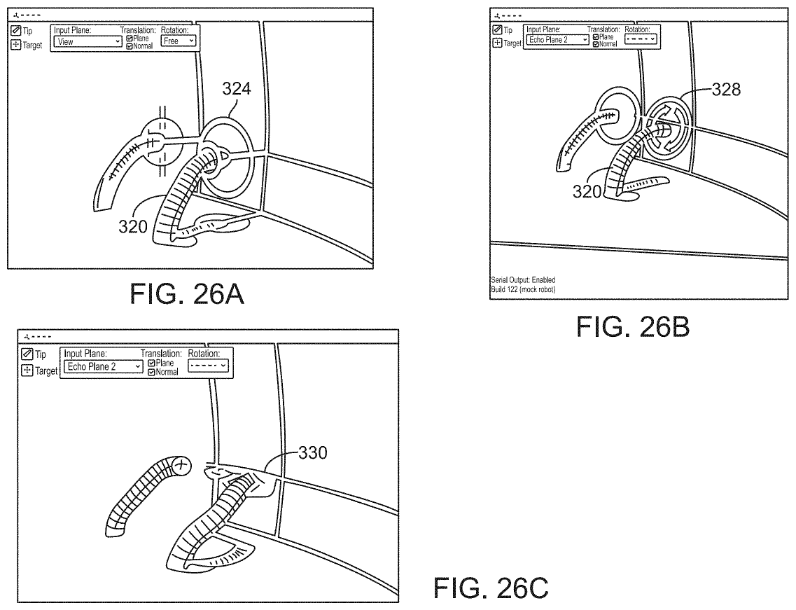

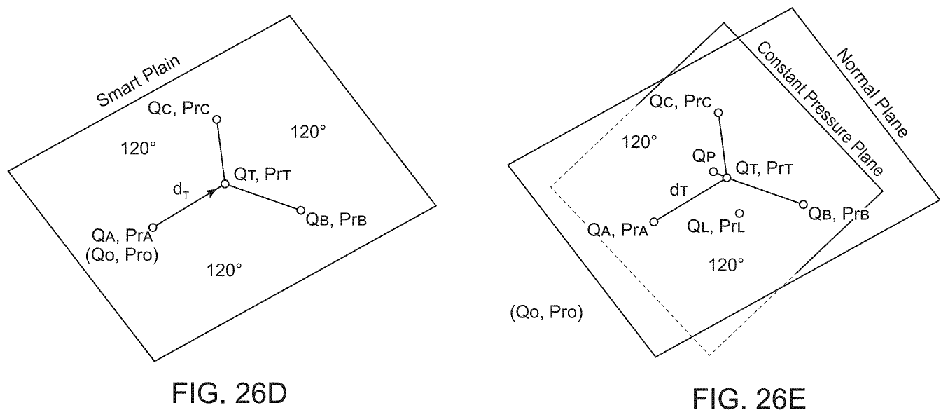

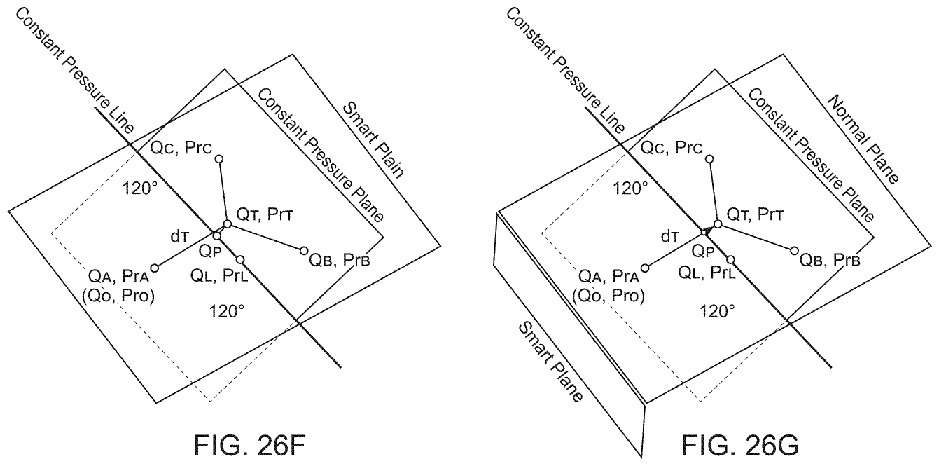

Referring now to FIGS. 1, 19 and 26A-26C, processor 214 can optionally provide a plurality of alternative constrained motion modes, and may superimpose indicia of movement available in those constrained modes in the auxiliary display image 252. For example, in FIG. 26A 3D virtual model image 320 in an unconstrained movement mode allows the user to input movement commands 212 using a 6 DOF input device 16. To graphically indicate to the user that movement commands in the display space 208 can induce translation of the catheter, rotation of the catheter, or combinations of both, an unconstrained movement widget 324 is superimposed on the tip of the 3D catheter model image in the auxiliary display image 252, with the unconstrained movement widget including a semi-transparent sphere indicative of allowability of rotation commands and an extended catheter axis indicative of allowability of translation commands. Optionally, corresponding 2D widgets may similarly be projected to one, some, or all of the 2D image planes and superimposed on the 2D virtual and/or captured catheter images.

Referring now to FIG. 26B, an advantageous plane-and-spin constrained mode allows the user to identify a plane, and then allows input commands to induce constrained movement of the catheter tip to the extent that the input commands induce: i) translation of the tool, tip, or receptacle along the identified plane, and/or ii) rotation of the tool, tip, or receptacle about an axis normal to the identified plane. The constraining of the input commands for this plane-and-spin mode (and other constrained movement modes of the system) are performed by a constraints module of processor 214, and a plane-and-spin widget indicative of this mode superimposed on the catheter image may comprise a disk centered on the tip or receptacle and parallel to the selected constraint plane, along with rotational arrows parallel to the disk. This plane-and-spin constrained movement mode is particularly advantageous for use with planar image acquisition systems as the user can select a 2D image plane. When movement commands are entered with the system in this mode using, for example, a 6 DOF input device 16, only the components of the input commands along the plane or rotational about the normal will induce movement of the catheter, so that the tip of the catheter can remain at a constant plane offset (the offset often being negligible so that the tip remains along the plane) and angular orientation relative to the plane (sometimes referred to herein as the same pitch relative to the plane), so that the user looking at a 2D image along that plane has good optical cues on which to evaluate movement and alignment with tissues that can be seen in that 2D image.

Referring now to FIG. 26C, a normal-and-pitch constrained movement mode (and associated normal-and-pitch constraint indicia 330) is largely complimentary to the plane-and-spin constrained mode described above. More specifically, when in this mode input commands are only considered to the extent that they are normal to the selected plane, and/or to the extent that the input commands seek changes in pitch, i.e., rotation about an axis parallel to the selected plane and perpendicular to the axis of the tip. When a user enters this mode catheter movement is inhibited in an acquired image along the selected plane (the movement being limited to movement into and out of the image plane, and/or to changes in pitch) so that if this mode is selected when the 2D image of the catheter is aligned with the 2D image of a target tissue the catheter will primarily remained aligned with that target tissue, somewhat giving the impression that the catheter is locked in alignment for the selected plane. The user will generally make use of this mode while viewing a 2D image at an angularly offset orientation from the selected plane or while viewing a 3D image of the catheter. The exemplary normal-and-pitch constrained indicia comprises an axis along the allowed motion normal to the selected plane, and rotational arrows about the pitch axis, with these pitch arrows being disposed on a cylindrical surface to help differentiate the spin arrow.

Regarding the functionality and data processing for which constraints module 326 is configured, the following section provides additional details.

Boundary and Constraint Control Mode Types & Function

Five distinct control modes of processor 214 are described herein for addressing workspace boundaries and other constraints and which may optionally be implemented in constraints module 326 (see FIG. 19). Input module 216 and simulation module 256 will often be used to generate input command signals for pressure control module 230, and constraints module 326 will optionally use the functionality described below to return telemetry back to the simulation and/or input command modules when the pressure control module determines that the commanded movement may encounter a workspace boundary, typically in the form of a pressure boundary when using the preferred articulation balloon array structures described above. While these control modes are often described below with reference to pressure limits and associated calculations, alternative embodiments may make use of torque limits or the like while similarly constraining motions to planes, lines, and the like. Similarly, while the exemplary simulation module 256 is implemented using machine-readable code in a Unity™ 3D graphical software environment (so that the description below may, for example, reference communications between the pressure controller and Unity™), it should be understood that alternative environments may be implemented using communications between the pressure control module 256 (sometimes referred to below as the Controller) and an alternative commercially available or proprietary 3D graphical software engine. The simulation module optionally uses spatial modes that are somewhat similar to those described below for controlling the catheter tip or tool receptacle of the therapy delivery system, but may not alter telemetry output from the pressure module back to the simulation (sometimes referenced below as telemetry) and so that processor 214 may benefit from the alternative constraint functionality described below to help pressure controller module 230 to meet the boundary limitations with appropriate telemetry back to the simulation module 256. The constraint control modes described herein include:
5D Shift/Scale Mode
3D Gradient Mode
Planar Mode
Line Mode
Gimbal Mode
Axis Mode
Segment Mode

Mode Table: Table 2 below describes the purpose and the general interaction between the input system 204, simulation module 256, and the pressure controller 230 (and particularly the response telemetry from the pressure controller to the simulation module).

TABLE 2

| Control | | Adjusted DOF | |
|---|---|---|---|
| Mode | Driven DOF | (at boundary) | Fixed DOF |
| 5D | All | 3D Position first, | None |
| Shift/Scale | | then Orientation | |
| 3D Gradient | All | 3D Position | None |
| Plane | Planar Position, | Planar Position | Normal Position |
| | 2D Rotation | | |
| Line | Normal Position, | Lineal Position | Planar Position |
| | 2D Rotation | | |
| Gimbal | 2D Rotation | 2D Rotation | 3D Position |
| Axial | 1D Rotation | None | 3D Position |
| | (Driven) | | 1D Rotation |
| | | | (Passive) |
| Segment | Drive Segment | Passive Segments | DePendent on |
| | (+other modes) | (+other modes) | other modes |

5D Shift/Scale vs. 3D Gradient

This is for motion unconstrained (other than pressure limits) in 5/6D space. The planar position can give way to achieve the orientation commanded. It is used with an unconstrained 6DOF input such as a Tango™ smartphone in free space and at pressure boundaries. There are two modalities described herein:

Shift/Scale Mode: This uses the Shift and Scale functions to respond to the pressure boundary; and

Gradient Mode: This function uses three points (A, B, C) with set orientations and in the vicinity of the goal Q (as defined by the input) to form a local linear 3D pressure gradient to estimate the goal position or the closest achievable position in the 3D space.

Planar Mode: This is for motion constrained to a plane. The planar position can give way to achieve the orientation commanded. It is used when, for example, the Tango™ 6DOF input system (sometimes referenced below as Tango™) constrains motion to a plane, when the mouse can be used for translation commands (optionally when the left button is held) to move on a plane, and optionally when the roller button is held and the mouse moves on a plane for changing orientation. This mode uses the three point (A-B-C) Planar function. (Note that the Roll function optionally utilizes Line mode).

Line Mode: This is for motion constrained to a Line. The line position can give way to achieve the orientation commanded. It can be used when Tango constrains motion to a line and optionally with the mouse Roll function.

Gimbal Mode: This is for motion constrained to a point in space. The point does not give way. Tip orientation will slide along the orientation boundary and find the closest Tip position and telemetry. This mode can be used when Tango™ constrains motion to a point and optionally when the mouse roller button is held for orientation control.

Axial Mode: This is for motion constrained to a point with rotating fixed to one axis in space. The point and axis do not give way, and the single driven orientation may be achievable while the tip remains on this point. The tip stops at the workspace (pressure) boundary. It can be used when simulation module 256 constrains motion to a point and a single axis.

Segment Mode: This is for driving motion on individual segments at segment transitions where one segment is driven to articulate and elongate. The passive segments respond in a manor preset by the user. For example, the passive segments may be set to hold their orientation and position relative to its own segment base. A second example is that passive segments may be set to stay on a point, trajectory, or plane. This mode can be driven by Tango™, a mouse, and other input forms. In this mode different segments may be set to behave with or without spatial constraints utilizing some of the properties in the previously listed Modes.

Simulation module 256 sends to the pressure control module 230 the input mode, input parameters, and the trajectory point(s). The input data is different for different modes as follows. Note that the Target Data and Command Data sets will often both utilize this input mode strategy.

Input Data
Shift/Scale—Mode, Parameters, Q_{Command}, Q_{Target}
3D Gradient—Mode, Parameters, Q_{A}, Q_{B}, Q_{C} (for both command and target set)
Planar Mode—Mode, Parameters, Q_{A}, Q_{B}, Q_{C} (for both command and target set)
Line Mode—Mode, Parameters, Q_{A}, Q_{B}, Q_{C} (for both command and target set)
Gimbal Mode—Mode, Parameters, Q_{A}, Q_{B}, Q_{C} (for both command and target set)
Axial Mode—Mode, Parameters, Q_{A}, Q_{B}, Q_{C} (for both command and target set)
Segment Mode—Mode, Parameters, TBD, but either Q (world space), j (joint space), or a combination

The pressure control module functions differently in each mode.

Pressure Control Module Function
Shift/Scale—Iterates 3 times for best solution, uses Shift and Scale as desired.
3D Gradient—Iterates 3 times, but only once on each of Q_{A}, Q_{B}, Q_{C} and then creates lumen pressure gradient to find Q_{T} or Q_{P} (closest achievable point in 3D space)
Planar Mode—Iterates 3 times, but only once on each of Q_{A}, Q_{B}, Q_{c} and then creates lumen pressure gradient to find Q_{T} or Q_{P} (closest achievable point on the Plane)
Line Mode—Iterates 3 times, but only once on each of Q_{A}, Q_{B}, Q_{C} and then creates lumen pressure gradient to find Q_{T} or Q_{P} (closest achievable point on the Line)
Gimbal Mode—Iterates 3 times, but only once on each of Q_{A}, Q_{B}, Q_{C} and then creates lumen pressure gradient to find Q_{T} or Q_{P} (closest achievable orientation while maintaining point location)
Axial Mode—Iterates 3 times, but only once on each of Q_{A}, Q_{B}, Q_{C} and then creates lumen pressure gradient to find Q_{T} or Q_{P} (closest achievable driven angle while maintaining passive angle)

The pressure control module 230 sends simulation module 256 the error conditions, boundary conditions and trajectory data for the command, phantom, and actual segments.

Trajectory Data
5D Shift/Scale—error, boundary, j_{Command}, j_{Phantom}, j_{Actual}
3D Gradient—error, boundary, j_{Command}, j_{Phantom}, j_{Actual}
Planar Mode—error, boundary, j_{Command}, j_{Phantom}, j_{Actual}
Line Mode—error, boundary, j_{Command}, j_{Phantom}, j_{Actual}
Gimbal Mode—error, boundary, j_{Command}, j_{Phantom}, j_{Actual}
Axial Mode—error, boundary, j_{Command}, j_{Phantom}, j_{Actual}
Segment Mode—error, boundary, j_{Command}, j_{Phantom}, j_{Actual}

Simulation module 256 uses the error conditions, boundary conditions, and trajectory data to proceed with the next action.

Setting Up the Pressure Gradient with Fixed Orientation

Scaling & Shifting Function Limitations

The pressure control module 230 optionally uses the kinematic equations to find a solution for the goal Q_{T}. The solution produces a pressure vector, Pr_{T}, based on the Jacobean of the current location. This solution does not account for workspace boundaries in the form of lumen pressure limits. When the Pr_{T} vector includes components outside the maximum and minimum pressure limits, two functions may be implemented to find the closest achievable solution. The first is to shift segment-based pressures values into range. Shifting maintains orientation at the sacrifice of position. When a segments lumen pressure range is too large to shift, a secondary function scales the individual segment-based pressures. The scaling changes both the position and the orientation from the goal Q_{T}. The result of Shifting and Scaling is that the telemetry produced tends towards the closest special position available sometimes maintaining and other times changing the tip orientation. This Shifting and Scaling can be functional while the Goal Q_{T} is only constrained by a pressure boundary, though it may not produce the goal orientation. Scaling may (at least in some cases) inherently change the segments' orientation.

Gradient Control

Gradient control is a method for finding the closest positional telemetry at engaging the boundary, with or without additional spatial constraints, while maintaining the goal orientation. The function finds the closest available position Q_{d} to the goal Q_{T}. This method is an alternative for the Shifting and Scaling functions for finding the Q trajectories. Either methods may to be utilized in the pressure control module code at different times; Shifting and Scaling for unconstrained orientation, and the Gradient Control for when maintaining goal orientation with (or without) spatial constraints is preferred.

General Model and Steps Towards Finding the Closest Gradient Solution

With reference to FIG. 26D, points T, A, B, and C are located on a plane that intersects the goal position Q_{T}. Optionally, it might also intersect a line formed between a start position and a goal position Q_{T}. The orientation of the Plane depends on the spatial constraints. With no spatial constraints or when constrained to a line, the plane resides on the trajectory line and the orientation is arbitrary, though there may be preferable orientations and it may be desirable to vary the orientation in subsequent cycles. When constrained to a plane (such as Smart Plane) the orientation is defined. Each point references unique position coordinates of associated Q vectors. The Q orientation values (alpha and beta) for each Q vector are the same which allows for three unique positions (X, Y, Z) at three different pressure values (per lumen). Simulation module 256 derives Q_{A}, Q_{B}, and Q_{C} tip vectors based the desired Q_{T} input, and sends the three Q vectors to the pressure control module. The pressure control module solves for each lumen pressure (Pr_{A}, Pr_{B}, Pr_{C}) for each Q position. A lumen pressure gradient between Q positions is generated. The lumen pressure vector at the target point (\vec{P}r_{T}) is solved. If \vec{Pr}_{T} has one or more lumen pressures outside the lumen pressure limits, the gradient equations are used to find the closest alternative Q that is within pressure limits and maintains orientation.

For displacement commands, the pressure control module allows the user to slide the Tip along the boundary and to achieve the closest solution. For pivoting commands, displacement occurs only when at a boundary. For both, the limit of travel is to the closest position while achieving the goal orientation by shifting on the along the boundary.

Symbols
Q_{O}—current tip position in world space.
O_{T} goal position (Tip input) in world space.
Q_{A}, Q_{B}, Q_{C}—on Plane; equal distance to Q_{T}; 120° offset from each other around Q_{T}; Optionally Q_{A} is on line formed from Q_{O} and Q_{T}.
\vec{Pr}—vector containing each lumen pressure at a specific Q position.
d_{T}—is the planar target offset.

Simulation Module/Pressure Control Module Communication

Simulation module 256 solves for the planar Q's (Q_{A}, Q_{B}, Q_{C}) based on Q_{T} and sends the three Q vectors to the pressure control module 230. The pressure control module solves for the three pressure vectors (Pr_{A}, Pr_{B}, Pr_{C}), produces the pressure gradients, solves for the lumen pressures Pr_{T} or Pr_{P}, and sends position telemetry Q_{T} or Q_{P} to the simulation module.

Gradient Model

The following Gradient Math occurs in the pressure control module after receiving the Q vectors from the simulation module. This gradient model applies to the 3D Gradient Mode, Planar Mode, and the Line Mode.

Spatial Plane

Find the plane formed by the positions of Q_{A}, Q_{B}, and Q_{C}, using the X, Y, & Z component.

C_{X0}*(X−X_{A})+C_{Y0}*(Y−Y_{A})+C_{Z0}*(Z−Z_{A})=0 (1)
C_{X0}, C_{Y0}, & C_{X0} are the plane constants associated with A, B, and C points. A vector formed by (C_{X0}, C_{Y0}, C_{Z0}) is perpendicular to plane.
   X_{A}, Y_{A}, Z_{A} are known position points of Q_{A} and can be any known point.
   X, Y, & Z are the Q position coordinate variables.

Find the plane constants using the cross-product, to find the perpendicular vector. V₁=B−A=[(X_{B}−X_{A}), (Y_{B}−Y_{A}), (Z_{B}−Z_{A})]

V₂=C−A=[(X_{C}−X_{A}),(Y_{C}−Y_{A}),(Z_{C}−Z_{A})]

V_{P0}=V₁×V₂=(C_{X0},C_{Y0},C_{Z0})

C_{X0}=(Y_{B}−Y_{A})*(Z_{C}−Z_{A})−(Y_{C}−Y_{A})*(Z_{B}−Z_{A})

C_{Y0}=(X_{C}−X_{A})*(Z_{B}−Z_{A})−(X_{B}−X_{A})*(Z_{C}−Z_{A})

C_{Z0}=(X_{B}−X_{A})*(Y_{C}−Y_{A})−(X_{C}−X_{A})*(Y_{B}−Y_{A})

From equation 1

C_{X0}*X+C_{Y0}*Y+C_{Z0}*Z=C_{X0}*X_{A}+C_{Y0}*Y_{A}+C_{Z0}*Z_{A}

PL₀=C_{X0}*X_{A}+C_{Y0}*Y_{A}+C_{Z0}*Z_{A} PL₀ is the ABC plain constant

Pressure Plane

We assume a linear gradient for the lumen pressure variance throughout the Q sample range. Use the following formulas to solve for the planer “C” constants.

C_{Xi}*X+C_{Yi}*Y+C_{Zi}*Z=Prᵢ [2]
“i” represents the lumen number; for a two-segment system “i” is from 1 through 6.
C_{Xi},C_{Yi}, & C_{Xi}, are the pressure constants to estimate the pressure of lumen “i”. A vector formed by (C_{Xi}, C_{Yi}, C_{Zi}) is perpendicular to the “i” lumen pressure plane.
X, Y, & Z are the Q position coordinate variables.
Prᵢ is the estimated pressure of lumen “i” at position X, Y, and Z.

For each of the three Q's defined by A, B, and C, a position (X, Y, Z) and Lumen Pressure (Prᵢ) are known. Set up equation and solve for constants for each lumen pressure.

$${C}_{Xi} * {X}_{A} + {C}_{Yi} * {Y}_{A} + {C}_{Zi} * {Z}_{A} = {Pr}_{Ai}$$

$${C}_{Xi} * {X}_{B} + {C}_{Yi} * {Y}_{B} + {C}_{Zi} * {Z}_{B} = {Pr}_{Bi}$$

$${C}_{Xi} * {X}_{C} + {C}_{Yi} * {Y}_{C} + {C}_{Zi} * {Z}_{C} = {Pr}_{Ci}$$

$$[ XYZ ] \cdot \vec{{C}_{i}} = \vec{{Pr}_{i}}$$

$$[ XYZ ] = ( \begin{matrix}{X}_{A} & {Y}_{A} & {Z}_{A} \\ {X}_{B} & {Y}_{B} & {Z}_{B} \\ {X}_{C} & {Y}_{C} & {Z}_{C}\end{matrix} )$$

$$\vec{{C}_{i}} = {[ XYZ ]}^{- 1} \cdot \vec{{Pr}_{i}}$$

Including additional Q points which can be retained from the previous cycle, an alternative may be implemented.

\vec{C}ᵢ=(XYZ·XYZ^{T})⁻¹·XYZ^{T}·\vec{Pr}ᵢ

Goal Position

Geometrically, Q_{T} is the average of Q_{A}, Q_{B}, and Q_{C} and expressed as follows:

\vec{Q}_{T} = (\vec{Q}_{A} + \vec{Q}_{B} + \vec{Q}_{C})/3
X_{T} = (X_{A} + X_{B} + X_{C})/3; Y_{T} = (Y_{A} + Y_{B} + Y_{C})/3; Z_{T} = (Z_{A} +
Z_{B} + Z_{C})/3

Goal Lumen Pressure

The pressure vector can be found through the “C” constants vector:

\vec{Pr}₁=\vec{C}₁·\vec{Q}_{T}

Use equation 2 (6 times for two segments) and solve for the target lumen pressures \vec{Pr}_{T} (at position Q_{T}).

If all lumen pressures at \vec{Pr}_{T} are within the pressure limits, use the current pressure vector. If one or more pressure components are outside the limits, solve for the closest position on the Smart Plane where all pressures are within the pressure limits.

3D Gradient Mode

Pressure Limit Points

Referring now to FIG. 26E, for lumens that are not within a pressure limit, find the point on the limit plane when intersected by a normal line also intersects the goal point at Q_{T}. If there are more than one lumen outside the pressure limit, and the all these normal limit points produce pressure vectors with a component outside the limit, then find the closest pressure limit point at the intersection of paired planes and search these for the closest achievable lumen pressure vector.

Goal Point Normal Line

The Vector normal to the pressure plane is a derivative of the plane definition.

\vec{V}_{Ni}=(V_{NXi},V_{NYi},V_{NZi})=(C_{Xi},C_{Yi},C_{Zi})

This vector may be normalized to make it a unit vector.

Normal Line Constants, Normal Line Equations, and Intersect Point Q_{di} (at Pressure limit),

Choose best axis by choosing largest absolute value of \vec{V}_{Ni} components, (C_{Xi}, C_{Yi}, C_{Zi}). If |C_{Xi}| is the largest, solve for Normal Line Constants with X as variable. If |C_{Yi}| is the largest, solve for Normal Line Constants with Y as variable. If |C_{Zi}| is the largest, solve for Normal Line Constants with Z as variable.

TABLE 3

| Vari- | | | |
|---|---|---|---|
| able | | | |
| Axis | X | Y | Z |
| ΔN_{Xi} | | V_{PXik}/V_{PYik} | V_{PXik}/V_{PZik} |
| N_{Xi} | | X_{Pi} − (V_{PXik}/V_{PYik})*Y_{Pi} | X_{Pi} − (V_{PXik}/ |
| | | | V_{PZik})*Z_{Pi} |
| ΔN_{Yi} | C_{Yi}/C_{Xi} | | V_{PYik}/V_{PZik} |
| N_{Yi} | Y_{T} − (C_{Yi}/C_{Xi})*X_{T} | | Y_{Pi} − (V_{PYik}/ |
| | | | V_{PZik})*Z_{Pi} |
| ΔN_{Zi} | C_{Zi}/C_{Xi} | V_{PZik}/V_{PYik} | |
| N_{Zi} | Z_{T} − (C_{Zi}/C_{Xi})*X_{T} | Z_{Pi} − (V_{PYik}/V_{PXik})*Y_{Pi} | |

Define the Intersection Line of each pressure limit plane. Use the same variable axis as with the TABLE 3 above. Line equations are formed from the equations of TABLE 4 below.

TABLE 4

| Vari- | | | |
|---|---|---|---|
| able | | | |
| Axis | X | Y | Z |
| X_{Pi} | X_{Pi} | N_{Xi} + ΔN_{Xi}*X_{Pi} | N_{Xi} + ΔN_{Xi}*Z_{Pi} |
| Y_{Pi} | N_{Yi} + ΔN_{Yi}*χ_{Pi} | Y_{Pi} | N_{Yi} + ΔN_{Yi}*Z_{Pi} |
| Z_{Pi} | N_{ZXi} + ΔN_{ZXi}*χ_{Pi} | N_{Zi} + ΔN_{Zi}*χ_{Pi} | Z_{Pi} |

Find the intersection point of the Normal line and the constant pressure plane, for each lumen that is not within the pressure limits.

Insert “X” line equations:

C_{Xi}*X_{Pi}+C_{Yi}*Y_{Pi}+C_{Zi}*Z_{Pi}=Pr_{Limit}

[from 2]

C_{Xi}*X_{Pi}+C_{Yi}*(N_{Yi}+N_{YXi}*X_{Pi})+C_{Zi}*(N_{Zi}+N_{ZXi}*X_{Pi})=Prᵢ

C_{Xi}*X_{Pi}+C_{Yi}*N_{Yi}+C_{Yi}*N_{YXi}*X_{Pi}+C_{Zi}*N_{Zi}+C_{Zi}*N_{ZXi}*X_{Pi}=Prᵢ

(C_{Xi}+C_{Yi}*N_{YXi}+C_{Zi}*N_{ZXi})*X_{Pi}+(C_{Yi}*N_{Yi}+C_{Zi}*N_{Zi})=Prᵢ

X_{Pi}=[Pr_{Limit}−(C_{Yi}*N_{Yi}+C_{Zi}*N_{Zi})]/(C_{Xi}+C_{Yi}*N_{YXi}+C_{Zi}*N_{ZXi})

Y_{Pi}=N_{Yi}+N_{YXi}*X_{Pi}

Z_{Pi}=N_{Zi}+N_{ZXi}*X_{Pi}

“Y” and “Z” line equations are similarly resolved.

TABLE 5

| Vari- | | | |
|---|---|---|---|
| able | | | |
| Axis | X | Y | Z |
| X_{Pi} | [Pr_{Limit} − (C_{Yi}*N_{Yi} + | X_{Pi} = N_{Xi} + ΔN_{Xi}*Y_{Pi} | N_{Xi} + ΔN_{Xi}*Z_{Pi} |
| | C_{Zi}*N_{Zi})]/(C_{Xi} + | | |
| | C_{Yi}*ΔN_{Yi} + | | |
| | C_{Zi}*ΔN_{Zi}) | | |
| Y_{Pi} | N_{Yi} + ΔN_{Yi}*χ_{Pi} | [Pr_{Limit} − (C_{Xi}*N_{Xi} + | N_{Yi} + ΔN_{Yi}*Z_{Pi} |
| | | C_{Zi}*N_{Zi})]/ (C_{Xi}*ΔN_{Xi} + | |
| | | C_{Yi} + C_{Zi}*ΔN_{Zi}) | |
| Z_{Pi} | N_{Zi} + ΔN_{Zi}*X_{P} | N_{Zi} + ΔN_{Zi}*X_{Pi} | [Pr_{Limit} − |
| | | | (C_{Xi}*N_{Xi} + |
| | | | C_{Yi}*N_{Yi})]/ |
| | | | (C_{Xi}*ΔN_{Xi} + |
| | | | C_{Yi}*ΔN_{Yi} + C_{Zi}) |

Plane Crossing Vector

\vec{V}_{Cik}=\vec{V}_{Ni}×\vec{V}_{Nk}=(V_{CXi},V_{CYi},V_{CZi})

(V_{CXik},V_{CYik},V_{CZik})=[(V_{NYi}*V_{NZk}−V_{NZi}*V_{NYk}),(V_{NZi}*V_{NXk}−V_{NXi}*V_{NZk}), (V_{NXi}*V_{NYk}−V_{NYi}*V_{NXk})]

This vector may be normalized to make it a unit vector.

Perpendicular Vector from Normal Point to Plane Crossing Vector

\vec{V}_{Pik}=\vec{V}_{Ni}×\vec{V}Cᵢₖ=(V_{PXi},V_{PYi},V_{PZi})

(V_{PXik},V_{PYik},V_{PZik})=[(V_{NYi}*V_{CZik}−V_{NZi}*V_{CYik}),(V_{NZi}*V_{CXik}−V_{NXi}*V_{CZik}), (V_{NXi}*V_{CYik}−V_{NYi}*V_{CXik})]

Perpendicular Line Constants, Equations, and Intersect point Q_{Pik} (at pressure limit),

Choose best axis by associated with the smallest absolute value of \vec{V}_{Pik} components, (V_{PXik}, V_{PYik}, V_{PZik}).

If |V_{PXik}| is the smallest, solve for perpendicular Line Constants with X as variable. If is the smallest, solve for perpendicular Line Constants with Y as variable. If |V_{PZik}| is the smallest, solve for perpendicular Line Constants with Y as variable.

TABLE 6

| Vari- | | | |
|---|---|---|---|
| able | | | |
| Axis | X | Y | Z |
| ΔK_{Xik} | | V_{PXik}/V_{PYik} | V_{PXik}/V_{PZik} |
| K_{Xik} | | X_{Pi} − (V_{PXik}/ | X_{Pi} − (V_{PXik}/ |
| | | V_{PYik})*Y_{Pi} | V_{PZik})*Z_{Pi} |
| ΔK_{Yik} | V_{PYik}/V_{PXik} | | V_{PYik}/V_{PZik} |
| K_{Yik} | Y_{Pi} − (V_{PYik}/V_{PXik})*X_{Pi} | | Y_{Pi} − (V_{PYik}/ |
| | | | V_{PZik})*Z_{Pi} |
| ΔK_{Zik} | V_{PZik}/V_{PXik} | V_{PZik}/V_{PYik} | |
| K_{Zik} | Z_{Pi} − (V_{PZik}/V_{PXik})*X_{Pi} | Z_{Pi} − (V_{PYik}/ | |
| | | V_{PXik})*Y_{Pi} | |

Find Line Intersection Point (X_{P}, Y_{P}, Z_{P}) on each plane intersection line. Use the same variable axis as with the Perpendicular Line Constants. Line equations can be formed from the following equivalencies.

“X” as variable input

K_{ZXik}+ΔK_{ZXik}*X_{Pi}=K_{Zki}+ΔK_{ZXki}*X_{Pi} or K_{YXik}+ΔK_{YXik}*X_{Pi}=K_{YXki}+ΔK_{YXik}*X_{Pi}

“Y” as variable input

K_{ZYik}+ΔK_{ZYik}*Y_{Pi}=K_{ZYki}+ΔK_{ZYki}*Y_{Pi} or K_{XYik}+ΔK_{XYik}*Y_{Pi}=K_{XYki}+ΔK_{XYki}*Y_{Pi}

“Z” as variable input

K_{YZik}+ΔK_{YZik}*Z_{Pi}=K_{Yki}+ΔK_{YZki}*Z_{Pi} or K_{Xik}+K_{XZik}*Z_{Pi}=K_{Xki}+K_{XZki}*Z_{Pi}

TABLE 7

| Vari- | | | |
|---|---|---|---|
| able | | | |
| Axis | X | Y | Z |
| X_{Pik} | (K_{Zki} − K_{Zik})/ | K_{Xik} + ΔK_{Xik}*Y_{P} | K_{Xik} + ΔK_{Xik}*Z_{P} |
| | (ΔK_{Zik} − ΔK_{Zki}) | | |
| Y_{Pik} | K_{Yik} + ΔK_{Yik}*X_{Pik} | (K_{Zki} − K_{Zik})/ | K_{Yik} + ΔK_{Yik}*Z_{P} |
| | | (ΔK_{Zik} − ΔK_{Zki}) | |
| Z_{Pik} | K_{Zik} + ΔK_{Zik}*X_{Pik} | K_{Zik} + ΔK_{Zik}*Y_{P} | (K_{Yki} − K_{Yik})/ |
| | | | (ΔK_{Yik} − ΔK_{Yki}) |

Solve the pressure array(s) for each plane intersection line Point

\vec{Pr}_{Ti}=\vec{C}ᵢ*(X_{Pi},Y_{Pi},Z_{Pi})

\vec{Pr}_{Tik}=\vec{C}ᵢ*(X_{Pik},Y_{Pik},Z_{Pik})

Solve for the intersect points on the line formed (by lumens beyond the pressure limit) from crossing limit planes. The number of points may be dictated by the number of lumens that cross a limit pressure. The max number of planes for two segments may be six.

In the Table 8 below the i and k indicate a specific lumen combination where i and k are not be the same number and all combinations can only be selected once.

TABLE 8

| | | i | | | | | |
|---|---|---|---|---|---|---|---|
| | | 1 | 2 | 3 | 4 | 5 | 6 |
| k | 1 | | x | x | x | x | x |
| | 2 | | | x | x | x | x |
| | 3 | | | | x | x | x |
| | 4 | | | | | x | x |
| | 5 | | | | | | x |
| | 6 | | | | | | |

This chart indicates the number of lumen plane intersect points as a function of the number of lumen lines in play. Note that with six lumen planes there are 15 intersect lines and associated points as indicated by the “x's”. Add this to the Normal line intersects, with six lumens over the pressure limit and a total of 21 intersect points (6 normal+15 plane line points) may benefit from being resolved.

Now find the closest (and achievable) point Q_{P} to the target Q_{T} (with all pressure values within the limits). There should be one intersect point with the Pr vector within the pressure limits. To optimize the search sequence, note the following:
An achievable normal intersection points will be closer than any achievable plane or line intersection points. The Normal intersection point comes from the normal line through the goal point.
The farthest Normal intersection point will generally be the closest Normal point that can be achieved (within the pressure limits). If it is not within the pressure limits, the closest point will be one of the lumens' intersect plane line points.
It may be possible, for a lumen which is not pressure limited across Q_{A}, Q_{B}, or Q_{C} to be an intersect limit line that defines the closest point.

Planar Mode

Pressure Limit points

Referring now to FIG. 26F, for lumens that are not within a pressure limit, find the points on lines A-B, B-C, and C-A that cross the limit pressure line. These points will all be in a line in space.

First, determine if pressure limit plane is normal to the A-B-C plane, in which case the pressure may be parallel to the allowable direction of motion and there may be no solution. For this condition return the previous telemetry.

Second, determine which limit points are farthest apart. This avoids using two points with the same pressure which leads to a non-zero divisor when finding the points.

ΔPr_{ABC}=Pr_{AB}−Pr_{BC}

ΔPr_{BCA}=Pr_{BC}−Pr_{CA}

ΔPr_{CAB}=Pr_{CA}−Pr_{AB}

MinΔPr=0.001—Other numbers may be determined empirically from trial and error or derived.

Flag=IF(MAX(APrAnc,ΔPr_{ABC},ΔPr_{BCA},ΔPr_{CAB})>MinΔPr,“Normal”, IF(AND(ABS(ΔPr_{ABC})>=ABS(ΔPr_{BCA}),(ΔPr_{ABC})>=ABS(ΔPr_{CAB})),“AB”, IF(ΔPr_{BCA}>=ABS(ΔPr_{CAB}),“BC”, “CA”)))

=IF(Flag=“Normal”,<return previous telemetry>, IF(Flag=“AB”, (X_{Di}, Y_{Di}, Z_{Di})=(Pr_{Limit}−Pr_{Ai})/(Pn3i- PrAi)*[(X_{B}, Y_{B}, Z_{B})−(X_{A}, Y_{A},Z_{A})](X_{A}, Y_{A}, Z_{A}), IF(Flag=“BC”, (X_{Di}, Y_{Di}, Z_{Di})−(Pr_{Limit}−Pr_{Bi})/(Pr_{Ci}−Pr_{Bi})*[(X_{C}, Y_{C}, Z_{C})−(X_{B}, Y_{B},Z_{B})]+(X_{B},Y_{B},Z_{B}), IF(Flag=“CE”, (X_{Di},Y_{Di},Z_{Di})=(Pr_{Limit}−Pr_{Ci})/(Pr_{Ai}−Pr_{Ci}^{)*}[(X_{A},Y_{A},Z_{A})−(X_{C},Y_{C},Z_{C})]+(X_{C},Y_{C},Z_{C}), <missing flag>))))

Limit Line Vector

Find the pressure Limit Line (unit) Vector with the cross product of the pressure and A-B-C plane's normal vectors, which can be taken directly from the plane constants above.

\vec{V}_{L}=(C_{Xi},C_{Yi},C_{Zi})×(C_{X0},C_{Y0},C_{Z0})=(V_{LXi},V_{LYi},V_{LZi})

(V_{LXi},V_{LYi},V_{LZi})=[(C_{Yi}*C_{Z0}−C_{Zi}*C_{Y0}),(C_{Zi}*C_{X0}−C_{Xi}*C_{Z0}),(C_{Xi}*C_{Y0}−C_{Yi}*C_{X0})]

This vector may be normalized to make it a unit vector.

Normal Line Vector

Normal Line (unit) Vector is the cross product of a line normal to the ABC plane with the Limit Line Vector.

\vec{V}Ni=\vec{V}_{Li}×\vec{C}ᵢ=(V_{NXi},V_{NYi},V_{NZi})

(V_{NXi},V_{NYi},V_{NZi})=[(V_{LYi}*C_{Z0}−V_{LZi}*C_{Y0}),(V_{LZi}*C_{X0}−V_{LXi}*C_{Z0}),(V_{LXi}*C_{Y0}−V_{LYi}*C_{X0})]

This vector may be normalized to make it a unit vector.

Limit Line Constants

Solve the Limit Line Constants by choosing the best variable axis. Look for maximum vector component value of the following equation.

\vec{V}_{Li}·\vec{V}_{Ni}

If(V_{LXi}·V_{NXi}=Max vector component, “X” is variable axis)

If(V_{LYi}·V_{NYi}=Max vector component, “Y” is variable axis)

If(V_{LZi}·V_{NZi}=Max vector component, “Z” as variable axis)

TABLE 9

| Vari- | | | |
|---|---|---|---|
| able | | | |
| Axis | X | Y | Z |
| ΔK_{Xi} | | V_{LXi}/V_{LYi} | V_{LXi}/V_{LZi} |
| K_{Xi} | | X_{Di} − (V_{LXi}/V_{LYi})*Y_{Di} | X_{Di} − (V_{LXi}/ |
| | | | V_{LZi})*Z_{Di} |
| ΔK_{Yi} | V_{LYi}/V_{LXi} | | V_{LYi}/V_{LZi} |
| K_{Yi} | Y_{Di} − (V_{LYi}/V_{LXi})*X_{Di} | | Y_{Di} − (V_{LYi}/ |
| | | | V_{LZi})*Z_{Di} |
| ΔK_{Zi} | V_{LZi}/V_{LXi} | V_{LZi}/V_{LYi} | |
| K_{Zi} | Z_{Di} − (V_{LZi}/V_{LXi})*X_{Di} | Z_{Di} − (V_{LYi}/V_{LXi})*Y_{Di} | |

Normal Line Constants

Use the same variable axis as with the Limit Line Constants.

TABLE 10

| Vari- | | | |
|---|---|---|---|
| able | | | |
| Axis | X | Y | Z |
| ΔN_{Xi} | | V_{NXi}/V_{NYi} | V_{NXi}/V_{NZi} |
| N_{Xi} | | X_{T} − (V_{NXi}/V_{NYi})*Y_{T} | X_{T} − (V_{NXi}/ |
| | | | V_{NZi})*Z_{T} |
| ΔN_{Yi} | V_{NYi}/V_{NXi} | | V_{NYi}/V_{NZi} |
| N_{Yi} | Y_{T} − (V_{NYi}/V_{NXi})*X_{T} | | Y_{T} − (V_{NYi}/ |
| | | | V_{NZi})*Z_{T} |
| ΔN_{Zi} | V_{NZi}/V_{NXi} | V_{NZi}/V_{NYi} | |
| N_{Zi} | Z_{T} − (V_{NZi}/V_{NXi})*X_{T} | Z_{T} − (V_{NZi}/V_{NYi})*Y_{T} | |

Normal Line Intersect Point

Find Normal Line Intersection Point (X_{Pi}, Y_{Pi}, Z_{Pi}) for each lumen Line crossing the pressure limit. Use the same variable axis as with the Limit Line Constants.

TABLE 11

| Vari- | | | |
|---|---|---|---|
| able | | | |
| Axis | X | Y | Z |
| X_{Pi} | (N_{Zi} − K_{Zi})/(ΔK_{Zi} − | K_{Xi} + ΔK_{Xi}*Y_{Pi} | K_{Xi} + ΔK_{Xi}*Z_{Pi} |
| | ΔN_{Zi}) | | |
| Y_{Pi} | K_{Yi} + ΔK_{Yi}*X_{Pi} | (N_{Zi} − K_{Zi})/(ΔK_{Zi} − | K_{Yi} + ΔK_{Yi}*Z_{Pi} |
| | | ΔN_{Zi}) | |
| Z_{Pi} | K_{Zi} + ΔK_{Zi}*X_{Pi} | K_{Zi} + ΔK_{Zi}*Y_{Pi} | (N_{Yi} − K_{Yi})/(ΔK_{Yi} − |
| | | | ΔN_{Yi}) |

Limit Lines Intersect Points

Find the pressure Limit Lines (i, k) Intersection points for lumens that cross pressure limit. Use the same variable axis as with the Limit Line Constants.

TABLE 12

| Vari- | | | |
|---|---|---|---|
| able | | | |
| Axis | X | Y | Z |
| X_{Pik} | (K_{ZXi} − K_{ZXk})/ | K_{XYk} + ΔK_{XYk}*Y_{Pik} | K_{XZk} + ΔK_{XZk}*Z_{Pik} |
| | (ΔK_{ZXk} − Δ_{KZXi}) | | |
| Y_{Pik} | K_{YXk} + ΔK_{YXk}* X_{Pik} | (K_{ZYi} − K_{ZYk})/ | K_{YZi} + ΔK_{YZi}*Z_{Pik} |
| | | (ΔK_{ZYk} − ΔK_{ZYi}) | |
| Z_{Pik} | K_{ZXk} + ΔK_{ZXk}* X_{Pik} | K_{ZYk} + ΔK_{ZYk}*Y_{Pik} | (K_{YZi} − K_{YZk})/ |
| | | | (ΔK_{YZK} − ΔK_{YZi}) |

Intersect Point Pressure Vectors

Solve the pressure array(s) for each Intersection Point

\vec{Pr}_{Ti}=\vec{C}ᵢ*(X_{Pi},Y_{Pi},Z_{Pi})

\vec{Pr}_{Tik}=\vec{C}ᵢ*(X_{Pik},Y_{Pik},Z_{Pik})

Solve for the intersect points of all limit lines. The number of limit lines will be dictated by the number of lumens that cross a limit pressure. The max number possible for two segments is six lumens lines.

In Table 13 below the i and k indicate a specific lumen combination where i and k are not be the same number and all combinations should be selected only once.

TABLE 13

| | | i | | | | | |
|---|---|---|---|---|---|---|---|
| | | 1 | 2 | 3 | 4 | 5 | 6 |
| k | 1 | | x | x | x | x | x |
| | 2 | | | x | x | x | x |
| | 3 | | | | x | x | x |
| | 4 | | | | | x | x |
| | 5 | | | | | | x |
| | 6 | | | | | | |

This chart indicates the number of lumen line intersect points as a function of the number of lumen lines in play. Note that with six lumen lines there are 15 intersect points as indicated by the “x's”. Add this to the Normal line intersects, with six lumens over the pressure limit a total of 21 intersect points (6 normal+15 lumen line) would benefit from being resolved.

Now find the closest (and achievable) point Q_{d} to the target Q_{T} (with all pressure values within the limits). There should be one or more intersect points that are within the pressure limits. To optimize the search sequence, note the following.
An achievable normal intersection points will be closer than any achievable lumen line intersection points. The Normal intersection point comes the normal line through the Target point.
The farthest Normal intersection point will generally be the closest Normal point that can be achieved (within the pressure limits). If it is not within the pressure limits, the closest point will be one of the lumen line intersection points.
It may be possible for a lumen which is not pressure limited across Q_{A}, Q_{B}, or Q_{c} to be an intersect limit line that defines the closest point.

Line Mode

Pressure Limit points

Referring now to FIG. 26G, for lumens that are not within a pressure limit, find the points on lines A-B, B-C, and C-A that cross the limit pressure line. These points will all be in a line in space.

First, determine if pressure limit plane is normal to the A-B-C plane, in which case the pressure parallel to the allowable direction of motion and there may be no solution. For this condition return the previous telemetry.

ΔPr_{ABC}=Pr_{AB}−Pr_{BC}

ΔPr_{BCA}=Pr_{BC}−Pr_{CA}

ΔPr_{CAB}=Pr_{CA}−Pr_{AB}

MinΔPr=0.001 (Other numbers may be determined empirically or analytically.)

Second, determine which limit points are farthest apart. This avoids using two points with the same pressure which leads to a non-zero divisor when finding the points.

Flag=IF(MAX(ΔPr_{ABC},ΔPr_{BCA},ΔPr_{CAB})<MinΔPr,“Normal”, IF(AND(ABS(ΔPr_{ABC})>=ABS(ΔPr_{BCA}),(ΔPr_{ABC})>=ABS(ΔPr_{CAB})),“AB”, IF(ΔPr_{BCA}>=ABS(ΔPr_{CAB}),“BC”, “CA”))) =IF(Flag=“Normal”,<return previous telemetry>, IF(Flag=“AB”(X_{Di},Y_{Di},Z_{Di})=(Pr_{Limit}−Pr_{Ai})/(Pr_{Bi}−Pr_{Ai})*[(X_{B},Y_{B},Z_{B})−(X_{A},Y_{A},Z_{A})]+(X_{A},Y_{A},Z_{A}), IF(Flag=“BC”, (X_{Di},Y_{Di},Z_{Di})=(Pr_{Limit}−Pr_{Bi})/(Pr_{Ci}−Pr_{Bi})*[(X_{C},Y_{C},Z_{C})−(X_{B},Y_{B},Z_{B})]+(X_{B},Y_{B},Z_{B}), IF(Flag=“CE”, (X_{Di},Y_{Di},Z_{Di})=(Pr_{Limit}−Pr_{Ci})/(Pr_{Ai}−Pr_{Ci})*[(X_{A},Y_{A},Z_{A})−(X_{C},Y_{C},Z_{C})]+(X_{C},Y_{C},Z_{C}), <missing flag>))))

Pressure Limit Line

Find the pressure Limit Line (unit) Vector with the cross product of the pressure and A-B-C plane's normal vectors, which can be taken directly from the plane constants above.

\vec{V}_{L}=(V_{LXi},V_{LYi},Z_{LZi})=(C_{Xi},C_{Yi},C_{Zi})×(C_{X0},C_{Y0},C_{Z0})

(V_{LXi},V_{LYi},V_{LZi})=[(C_{Yi}*C_{Z0}−C_{Zi}*C_{Y0}),(C_{Zi}*C_{X0}−C_{Xi}*C_{Z0}),(C_{Xi}*C_{Y0}−C_{Yi}*C_{X0})]

This vector may be normalized to make it a unit vector.

Normal Line Vector

Normal Line (unit) Vector is the cross product of a line normal to the ABC plane with the Limit Line Vector.

\vec{V}N=(V_{NX},V_{NY},V_{NZ})=[(X_{A}−X_{T}),(Y_{A}−X_{T}),(Z_{A}−Z_{T})]

This vector may be normalized to make it a unit vector.

Limit Line Constants

Solve the Limit Line Constants by choosing the best variable axis. Look for maximum vector component value of the following equation.

\vec{V}_{Li}·\vec{V}_{N}

If(V_{LXi}·V_{NX}=Max vector component, “X” is variable axis)

If(V_{LYi}·V_{NY}=Max vector component, “Y” is variable axis)

If(V_{LZi}·V_{NZ}=Max vector component, “Z” as veriable axis)

Limit line Constants:

TABLE 14

| Vari- | | | |
|---|---|---|---|
| able | | | |
| Axis | X | Y | Z |
| ΔK_{Xi} | | V_{LXi}/V_{LYi} | V_{LXi}/V_{LZi} |
| K_{Xi} | | X_{Di} − (V_{LXi}/V_{LYi})*Y_{Di} | X_{Di} − (V_{LXi}/ |
| | | | V_{LZi})*Z_{Di} |
| ΔK_{Yi} | V_{LYi}/V_{LXi} | | V_{LYi}/V_{LZi} |
| K_{Yi} | Y_{Di} − (V_{LYi}/V_{LXi})*X_{Di} | | Y_{Di} − (V_{LYi}/ |
| | | | V_{LZi})*Z_{Di} |
| ΔK_{Zi} | V_{LZi}/V_{LXi} | V_{LZi}/V_{LYi} | |
| K_{Zi} | Z_{Di} − (V_{LZi}/V_{LXi})*X_{Di} | Z_{Di} − (V_{LYi}/V_{LXi})*Y_{Di} | |

Normal Line Constants

Solve Normal Line Constants (line normal to Smart Plane). Use the same variable axis as with the Limit Line Constants.

TABLE 15

| Vari- | | | |
|---|---|---|---|
| able | | | |
| Axis | X | Y | Z |
| ΔN_{X} | | V_{NX}/V_{NY} | V_{NX}/V_{NZ} |
| N_{X} | | X_{T} − (V_{NX}/V_{NY})*Y_{T} | X_{T} − (V_{NX}/ |
| | | | V_{NZ})*Z_{T} |
| ΔN_{Y} | V_{NY}/V_{NX} | | V_{NY}/V_{NZ} |
| N_{Y} | Y_{T} − (V_{NY}/V_{NX})*X_{T} | | Y_{T} − (V_{NY}/ |
| | | | V_{NZ})*Z_{T} |
| ΔN_{Z} | V_{NZ}/V_{NX} | V_{NZ}/V_{NY} | |
| N_{Z} | Z_{T} − (V_{NZ}/V_{NX})*X_{T} | Z_{T} − (V_{NZ}/V_{NY})*Y_{T} | |

Find Normal Line and pressure Limit Lines intersection points (X_{Pi}, Y_{Pi}, Z_{Pi}) for each lumen Line crossing the pressure limit. Use the same variable axis as with the Limit Line Constants.

TABLE 16

| Vari- | | | |
|---|---|---|---|
| able | | | |
| Axis | X | Y | Z |
| X_{Pi} | (N_{Zi} − K_{Zi})/(ΔK_{Zi} − | K_{Xi} + ΔK_{Xi}*Y_{Pi} | K_{Xi} + ΔK_{Xi}*Z_{Pi} |
| | ΔN_{Zi}) | | |
| Y_{Pi} | K_{Yi} + ΔK_{Yi}*X_{Pi} | (N_{Zi} − K_{Zi})/(ΔK_{Zi} − | K_{Yi} + ΔK_{Yi}*Z_{Pi} |
| | | ΔN_{Zi}) | |
| Z_{Pi} | K_{Zi} + ΔK_{Zi}*X_{Pi} | K_{Zi} + ΔK_{Zi}*Y_{Pi} | (N_{Yi} − K_{Yi})/(ΔK_{Yi} − |
| | | | ΔN_{Yi}) |

Solve the pressure array(s) for each Intersection Point

\vec{Pr}_{Ti}=\vec{C}ᵢ*(X_{Pi},Y_{Pi},Z_{Pi})

Solve for the intersect points of all limit lines. The number of limit lines will be dictated by the number of lumens that cross a limit pressure. The max number for two segments may be six lumens lines.

Now find the closest (achievable) point Q_{d} to the target Q_{T} (with all pressure values within the limits). There should be one or more intersect points that are within the pressure limits.

Setting Up the Pressure Gradient with Fixed Position

General Model and Steps Towards Finding the Closest Gradient Solution

Referring once again to FIG. 26D, points T, A, B, and C are located at the same position (graphically separated for conceptualizing) but with different spin (α) and pitch (β) angles than the goal vector Q_{T}. The commanded rotation is defined in the direction from Q_{A} to Q_{T}. Each tip point has unique orientation associated with the Q vectors. The Q position values (X, Y, Z) for each Q vector are the same which allows for two unique orientation (βₓ, β_{y}) at three different pressure values (per lumen). The simulation module derives Q_{A}, Q_{B}, and Q_{C} tip vectors based the desired Q_{T} input, and sends the three Q vectors to the pressure control module. The pressure control module solves for the lumen pressure (Pr_{A}, Pr_{B}, Pr_{C}) for each Q position. A lumen pressure gradient between the Q positions is generated. The lumen pressure vector at the target point (\vec{Pr}_{T}) is solved using this gradient. If \vec{Pr}_{T} has one or more lumen pressures outside the lumen pressure limits, the gradient equations are used to find the closest alternative orientation (βₓ, β_{y}) that is within pressure limit.

Symbols
Q_{O}—current tip position in world space.
Q_{T}—goal orientation (Tip Input) in world space.
Q_{A}, Q_{B}, Q_{C}—same Q_{T} (X,Y,Z) position; equal angular displacement from Q_{T}.
\vec{Pr}—vector containing each lumen pressure at a specific Q position.
d_{T}—is the orientational target offset.

Gradient Model

The following Gradient Math occurs in the pressure control module after receiving the

Q vectors from the simulation module. This gradient model applies to the Gimbal and Axial Mode.

Spatial Plane

Find the plane formed by the positions of Q_{A}, Q_{B}, and Q_{C}, using the βₓ, and β_{y}, component.

C_{X0}*(βx−βx_{A})+C_{Y0}*(βy−βy_{A})=0 C_{X0}*βx+C_{Y0}*βy=C_{X0}*βx_{A}+C_{Y0}*βy_{A} (1)
C_{X0}, and C_{Y0}, are the plane constants (C_{Z0}=0) associated with points A, B, and C. A vector formed by (C_{X0}, C_{Y0}, C_{Z0}) is perpendicular to plane.
X_{A}, Y_{A}, Z_{A} are known position points of Q_{A} and can be any known point.
X, Y, & Z are the Q position coordinate variables.

Pressure Plane

Linear gradient for the lumen pressure variance throughout the Q sample range. Use the following formulas to solve for the planer “C” constants.

C_{Xi}*βx+C_{Yi}*βy=Prᵢ [2]
“i” represents the lumen number; for a two-segment system “i” is from 1 through 6.
C_{Xi} and C_{Yi} are the pressure constants to estimate the pressure of lumen “i”.
βx and βy are the Q orientation coordinate variables.
Prᵢ is the estimated pressure of lumen “i” at orientation βx and βy.

For each of the three Q's defined by A, B, and C, two orientations (βxᵢ, βyᵢ) and Lumen Pressure (Prᵢ) are known. Set up equation and solve for constants for each lumen pressure.

$${C}_{Xi} * β {x}_{A} + {C}_{Yi} * β {y}_{A} = {Pr}_{Ai}$$

$${C}_{Xi} * β {x}_{B} + {C}_{Yi} * β {y}_{B} = {Pr}_{Bi}$$

$${C}_{Xi} * β {x}_{C} + {C}_{Yi} * β {y}_{C} = {Pr}_{Ci}$$

$$[ β ] \cdot \vec{{C}_{i}} = \vec{{Pr}_{i}}$$

$$[ β ] = ( \begin{matrix}β {x}_{A} & β {y}_{A} \\ β {x}_{B} & β {y}_{B} \\ β {x}_{C} & β {y}_{C}\end{matrix} )$$

$$\vec{{C}_{i}} = {[ β ]}^{- 1} \cdot \vec{{Pr}_{i}}$$

Since there are more Q points than variables, a least square fit may be applied for a pseudo inverse matrix.

\vec{C}ᵢ=[β]·[β]^{T})⁻¹·[β]^{T}·\vec{Pr}ᵢ

Goal Position

Geometrically, Q_{T} is the average of Q_{A}, Q_{B}, and Q_{C} and expressed as follows:

\vec{Q}_{T}=(\vec{Q}_{A}+\vec{Q}_{B}+\vec{Q}_{C})/3

βx_{T}=(βx_{A}+βx_{B}+βx)/3; βy_{T}=(βy_{A}+βy_{B}+βy_{C})/3

Goal Lumen Pressure

The pressure vector can be found through the “C” constants vector:

\vec{Pr}₁=\vec{C}_{I}·Q_{T}

Use equation 2 (6 times for two segments) and solve for the target lumen pressures \vec{Pr}_{T} (at position Q_{T}).
If all lumen pressures at \vec{Pr}_{T} are within the pressure limits, use the current pressure vector. If one or more pressure components are outside the limits, solve for the closest position on the Smart Plane where all pressures are within the pressure limits.

Pressure Limit Points

For lumens that are not within a pressure limit, find the points on graphic lines A-B, B-C, and C-A that cross the limit pressure line. These points will all be one a graphic line.

First, determine if pressure limit plane is normal to the A-B-C plane, in which case the pressure parallel to the allowable direction of motion and there may be no solution. For this condition return the previous telemetry.

ΔPr_{ABC}=Pr_{AB}−Pr_{BC}

ΔPr_{BCA}=Pr_{BC}−Pr_{CA}

ΔPr_{CAB}=Pr_{CA}−Pr_{AB}

MinΔPr=0.001 (Other numbers may be determined empirically or analytically.)

Second, determine which limit points are farthest apart. This avoids using two points with the same pressure which leads to a non-zero divisor when finding the points.

Flag=IF(MAX(ΔPr_{ABC},ΔPr_{BCA},ΔPr_{CAB})<MinΔPr,“Normal”, IF(AND(ABS(ΔPr_{ABC})>=ABS(ΔPr_{BCA}),(ΔPr_{ABC})>=ABS(ΔPr_{CAB})),“AB”, IF(ΔPr_{BCA}>=ABS(ΔPr_{CAB}),“BC”, “CA”)))

IF(Flag=“Normal”,<return previous telemetry>,

IF(Flag=“AB”, (βx_{Di},βy_{Di})=(Pr_{Limit}−Pr_{Ai}/(Pr_{Bi}−Pr_{Ai})*[(βx_{B},βy_{B})−(βx_{A},βy_{A})]+(βy_{A},βy_{A}),

IF(Flag=“BC”, (βx_{Di},βy_{Di})=(Pr_{Limit}−Pr_{Bi})/(Pr_{Ci}−Pr_{Bi})*[(βx_{C},βy_{C})−(βx_{B},βy_{B})]+(βx_{B},(βy_{B}),

IF(Flag=“CE”, (βx_{Di},βy_{Di})=(Pr_{Limit}−Pr_{Ci})/(Pr_{Ai}−Pr_{Ci})*[(βx_{A},βy_{A})−(βx_{C},βy_{C})]+(βx_{C},βy_{C}),

<missing flag>))))

Pressure Limit Line

Find the pressure Limit Line Vector. The Limit Line Vector is perpendicular to the Pressure constants Vector (C_{Xi}, C_{Yi}). Since they are perpendicular, the dot product of the “C” Vector and Limit Line Vector is equal to zero.

\vec{V}_{Li}·\vec{C}ᵢ=[V_{LXi},V_{LYi}]·(C_{Xi},C_{Yi})=0

\vec{V}_{Li}=[V_{LXi},V_{LYi}]=(C_{Yi},−C_{Xi})

This vector may be normalized to make it a unit vector.

Gimbal Mode

Gimbal Mode control allows change in two axes of orientations at a fixed position.

When orientation adjustments meet a boundary, the rotation slides along the angle boundary. The method maintains telemetry on a point while moving to the closest orientation.

Normal Line Vector

In Gimbal Mode, the Normal Line Vector is a line that passes through the goal point Q_{T} and is perpendicular to the Limit Line Vector. Since they are perpendicular, the dot product of the Normal Line Vector and Limit Line Vector is equal to zero.

\vec{V}_{Li}·\vec{V}_{Ni}=[C_{Yi},−C_{Xi}]·(V_{NXi},V_{NYi})=0

\vec{V}_{Ni}=(V_{NXi},V_{NYi})=(C_{Xi},C_{Yi})

This vector may be normalized to make it a unit vector.

Limit Line Constants

Solve the Limit Line Constants by choosing the best variable axis. Look for maximum vector component value of the following equation.

\vec{V}_{Li}·\vec{V}_{Ni}

If(V_{LXi}·V_{NXi}=Max vector component, “βx” is variable axis)

If(V_{LYi}·V_{NYi}=Max vector component, “βx” is variable axis)

Limit line Constants:

TABLE 17

| Vari- | | |
|---|---|---|
| able | | |
| Axis | βx | βy |
| ΔK_{Xi} | | V_{LXi}/V_{LYi} |
| K_{Xi} | | X_{Di} − (V_{LXi}/V_{LYi})*Y_{Di} |
| ΔK_{Yi} | V_{LYi}/VL_{Xi} | |
| K_{Yi} | Y_{Di} − (V_{LYi}/V_{LXi})*X_{Di} | |

Normal Line Constants

Solve Normal Graphic Line Constants. Use the same variable axis as with the Limit Line Constants.

TABLE 18

| Vari- | | |
|---|---|---|
| able | | |
| Axis | βx | βy |
| ΔN_{Xi} | | V_{NXi}/V_{NYi} |
| N_{Xi} | | X_{T} − (V_{NXi}/V_{NYi})*Y_{T} |
| ΔN_{Yi} | V_{NYi}/V_{NXi} | |
| N_{Yi} | Y_{T} − (V_{NYi}/V_{NXi})*X_{T} | |

Normal Line Intersect Point

Find Normal Line and pressure Limit Lines intersection points (βx_{Pi}, βy_{Pi}) for each lumen Line crossing the pressure limit. Use the same variable axis as with the Limit Line Constants.

TABLE 19

| Vari- | | |
|---|---|---|
| able | | |
| Axis | βx | βy |
| βx_{Pi} | (N_{Yi} − K_{Yi})/(ΔK_{Yi} − | K_{Xi} + ΔK_{Xi}*Y_{Pi} |
| | ΔN_{Yi}) | |
| βy_{Pi} | K_{Yi} + ΔK_{Yi}*X_{Pi} | (N_{Xi} − K_{Xi})/(ΔK_{Xi} − |
| | | ΔN_{Xi}) |

Limit Lines Intersect Points

Find the pressure Limit Lines (i, k) Intersection points for lumens that cross pressure limit. Use the same variable axis as with the Limit Line Constants.

TABLE 20

| Vari- | | |
|---|---|---|
| able | | |
| Axis | βx | βy |
| βx_{Pik} | (K_{ZXi} − K_{ZXk})/(ΔK_{ZXk} − | K_{XYk} + ΔK_{YXk}*Y_{Pik} |
| | ΔK_{ZXi}) | |
| βy_{Pik} | K_{YXk} + ΔK_{YXk}* X_{Pik} | (K_{ZYi} − K_{ZYk})/(ΔK_{ZYk} − |
| | | ΔK_{ZYi}) |

Intersect Point Pressure Vectors

Solve the pressure array(s) for each Intersection Point

\vec{Pr}_{Ti}=\vec{C}ᵢ*(βx_{Pi},σy_{Pi})

\vec{Pr}_{Tik}=\vec{C}ᵢ*(βx_{Pik},βy_{Pik})

Solve for the intersect points of all limit lines. The number of limit lines will be dictated by the number of lumens that cross a limit pressure. The max number possible for two segments is six lumens lines.

In the chart below the i and k indicate a specific lumen combination where i and k are not be the same number and all combinations should only be selected once.

TABLE 21

| | | i | | | | | |
|---|---|---|---|---|---|---|---|
| | | 1 | 2 | 3 | 4 | 5 | 6 |
| k | 1 | | x | x | x | x | x |
| | 2 | | | x | x | x | x |
| | 3 | | | | x | x | x |
| | 4 | | | | | x | x |
| | 5 | | | | | | x |
| | 6 | | | | | | |

Table 21 indicates the number of lumen line intersect points as a function of the number of lumen lines in play. Note that with six lumen lines there are 15 intersect points as indicated by the “x's”. Add this to the Normal line intersects, with six lumens over the pressure limit a total of 21 intersect points (6 normal+15 lumen line) would benefit from being resolved.

Now find the closest (and achievable) point Q_{d} to the target Q_{T} (with all pressure values within the limits). There should be one or more intersect points that are within the pressure limits. To optimize the search sequence, note the following.
An achievable normal intersection points will be closer than any achievable lumen line intersection points. The Normal intersection point comes the normal line through the Target point.
The farthest Normal intersection point will always be the closest Normal point that can be achieved (within the pressure limits). If it is not within the pressure limits, the closest point will be one of the lumen line intersection points.
It may be possible, though unlikely, for a lumen which is not pressure limited across Q_{A}, Q_{B}, or Q_{C} to be an intersect limit line that defines the closest point. The current math does not consider this condition and assumes this will not occur.

Axial Mode

Axial Mode control allows change in orientation at a fixed position and about one axis. When orientation adjustments meet a boundary, pitch is sacrificed in order to meet circumferential angle about the Normal axis. The method maintains telemetry on a point while moving to the closest orientation while sacrificing pitch angle.

Normal Line Vector

For the Axial Mode the Normal Line Vector is the line normal to the ABC plane and, assuming point A is on trajectory path, it can be found by the orientation vector between point A and T.

\vec{V}_{N}=(V_{NX},V_{NY})=[(βx_{A}−βx_{T}),(βy_{A}−βy_{T})]

This vector may be normalized to make it a unit vector.

Limit Line Constants

Solve the Limit Line Constants by choosing the best variable axis. Look for maximum vector component value of the following equation.

\vec{V}_{Li}·\vec{V}_{N}

If(V_{LXi}·V_{NX}=Max vector component, “βx” is variable axis)

If(V_{LYi}·V_{NY}=Max vector component, “βy” is variable axis)

Limit line Constants:

TABLE 22

| Vari- | | |
|---|---|---|
| able | | |
| Axis | βx | βy |
| ΔK_{Xi} | | V_{LXi}/V_{LYi} |
| K_{Xi} | | X_{Di} − (V_{LXi}/V_{LYi})*Y_{Di} |
| ΔK_{Yi} | V_{LYi}/V_{LXi} | |
| K_{Yi} | Y_{Di} − (V_{LYi}/V_{LXi})*X_{Di} | |

Normal Line Constants

Solve Normal Graphic Line Constants. Use the same variable axis as with the Limit Line Constants.

TABLE 23

| Vari- | | |
|---|---|---|
| able | | |
| Axis | βx | βy |
| ΔN_{X} | | V_{NX}/V_{NY} |
| N_{X} | | X_{T} − (V_{NX}/V_{NY})*Y_{T} |
| ΔN_{Y} | V_{NY}/V_{NX} | |
| N_{Y} | Y_{T} − (V_{NY}/V_{NX})*X_{T} | |

Find Normal Line and pressure Limit Lines intersection points (X_{Pi}, Y_{Pi}, Z_{Pi}) for each lumen Line crossing the pressure limit. Use the same variable axis as with the Limit Line Constants.

TABLE 24

| Vari- | | |
|---|---|---|
| able | | |
| Axis | βx | βy |
| βx_{Pi} | (N_{Yi} − K_{Yi})/(ΔK_{Yi} − | K_{Xi} + ΔK_{Xi}*Y_{Pi} |
| | ΔN_{Yi}) | |
| βy_{Pi} | K_{Yi} + ΔK_{Yi}*X_{Pi} | (N_{Xi} − K_{Xi})/(ΔK_{Xi} − |
| | | ΔN_{Xi}) |

Solve the pressure array(s) for each Intersection Point

\vec{Pr}_{Ti}=\vec{C}ᵢ*(βx_{Pi},βy_{Pi})

Now find the closest (achievable) point Q_{d} to the target Q_{T} (with all pressure values within the limits). There should be one or more intersect points that are within the pressure limits.

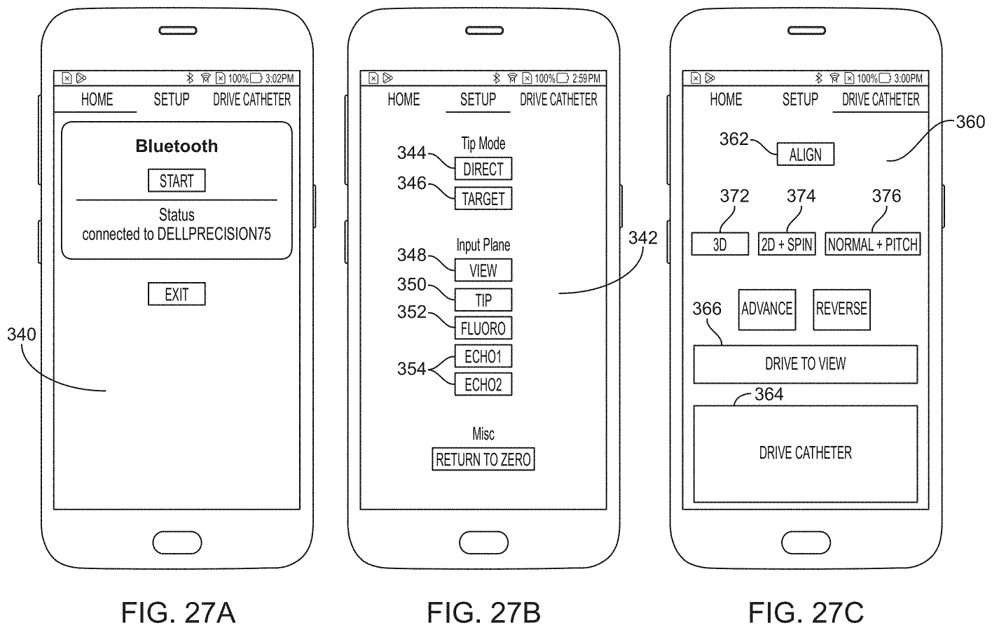

Referring now to FIGS. 1 and 27A-27C user interface pages of a mobile computing device configured for use as 6 DOF input 16 are shown. The exemplary mobile computing device comprises a Tango™-compatible ASUS ZenfoneAR™ running an Android™ operating system, although alternative augmented reality (AR) capable mobile computing devices configured for ARCore™, ARKit™, and/or other AR packages. A Home page 340 shown in FIG. 27A includes buttons associated with establishing or terminating Bluetooth™, WiFi, or other wireless communications with other components of the processor, with the buttons and underlying functionality being configured with security and identification protocols that inhibit malicious or inadvertent interference with the use of the articulation system. A Setup page 342 includes alternatively selectable Direct mode button 344 and Target mode button 346 that can be used to alternate the processor mode between a Drive mode that is configured for real-time driving of the catheter in response to movement commands, and a Target mode that is configured for driving of a virtual or phantom catheter, as described above. Setup page 342 also includes a number of alternatively selectable buttons associated with planes to which articulation modes may be constrained. The input plane buttons include a View plane button 348 which references the plane of display 206 (see FIG. 19). A Tip plane button 350 references a plane normal to the tip of the catheter. A flouro plane button 352 references a 2D image capture plane of the fluoro system, while Echo plane buttons 354 each reference a 2D image plane of the flouro system.

Referring now to FIG. 27C, Drive Catheter page 360 includes an Align button 362 which is configured to align the input space of the input device 16 with the display reference frame 208. For example, the user can orient the top end of the mobile computing device toward (parallel to) the display plane with the screen of the mobile device oriented upward and the elongate axis of the mobile device perpendicular to the display plane, and then engage the Align button. The orientation of the mobile device during engagement of the Align button can be stored, and subsequent input commands entered by, for example, engaging a Drive Catheter button 364 and moving the mobile device from a starting location and orientation (with the Drive Catheter button engaged) can be transformed to the display frame using standard quaternion operations (regardless of the specific starting orientation and location). Processor 214 can use this input to induce movement of the catheter (or a phantom catheter) as seen in an image shown in the display, to translate and rotate in correlation with the movement of the mobile device. Release of the Drive Catheter button can then decouple the input device 16 from the catheter. A Drive View button 366, when engaged, provides analogous coupling of the mobile device to a 2D, 3D, or hybrid 2D/3D image presented on the display so that the image (including the visible portions of the catheter and the displayed tissue) translate and rotate in correlation with movement of the mobile device as if the mobile device was coupled to the catheter tip, thereby allowing the user to view the image from different locations and/or orientations. Advance and retract buttons 368, 370 induce movement of the catheter along a trajectory between a first pose and a second or phantom pose, as described above. Alternatively selectable mode buttons, including a 3D mode button 372, a Planar-and-spin mode button 374, and a Normal-and-pitch mode button 376 can be used to select between unconstrained motion and motion constrained relative to the plane selected on the Setup page 342 as described above with reference to FIG. 27B.

Referring now to FIGS. 16A-16D, 19, and 25, the system 202 will often have an overall processor 214 that includes a first module such as an input command module 216 configured to receive input from the user for moving a virtual image 146 of the elongate body from a first pose 140 to a second pose 142 on the display 130. The processor will often also have a second module (such as a second input module 216) configured to receive a movement command, and in response, to drive actuators (see, e.g., balloons 42 in FIGS. 2 and 3A-3C) so as to move the elongate body along a trajectory 150 between the first pose and the second pose.

As seen in FIG. 19, system 202 will typically include one or more image capture system coupled to the display, such as flouro system 236 and/or echo system 238 (typically with an ICE probe, a TEE probe, a trans-thoracic echocardiograpy (TTE) probe, and/or the like). The input module 230, simulation module 256, and pressure control module 230 can work together to move the virtual image 146 of the receptacle and elongate body relative to a stored image of the internal surgical site shown on display 150. One or more of these components of the processor can be configured to transmit image capture commands to the image capture system in response to the same command used to induce movement of the actual elongate body along the trajectory, so that the image capture system selectively images the elongate body only shortly before initiating movement along the trajectory, during some or all of the time the elongate body is between the starting and stopping poses, and/or just after the elongate body has reached the desired pose. By establishing the target pose with reference to one or more still image and then acquiring imaging (particularly fluoro imaging) associated with the move irradiation of the patient, system user, and any other nearby medical professionals can be significantly reduced (as compared to other approaches). Optionally, by superimposing the virtual image on the display of the actual elongate body the user may be presented with a continuously available basis for image guidance and movement verification despite only intermittently imaging the elongate body between the poses. Note that despite such intermittent imaging the processor can still take advantage of image processing module 234 to track the movement of the elongate body using the intermittent images and the virtual image.

Referring now to FIGS. 16A-16D, 19, and 21, and 25, the system may optionally include a first image capture device (such as fluoro system 236) and a second image capture device (such as echo system 238) for generating first image data and second image data, respectively. To register display 206 to both image capture devices processor 214 may include a first registration module (optionally making use of select elements of constraint module 326) and a second registration module (again making use of elements of constraint module 326). Preferably, the first module will be configured for aligning the virtual image 144 of the elongate body with the first image of the elongate body, such as by translating (X-Y-Z) a distal tip of the virtual image on the display into alignment with the image of the actual catheter, spinning the virtual image into alignment, aligning a pitch of the virtual image, and rolling the virtual image, with some or all of the individual axes alignments being independent of the other axes alignments. The second registration module can be configured for aligning the second image of the elongate body with the virtual image, allowing independent modification of the registration of the second image modality without altering the registration of the first imaging modality. Once again, the second module may allow independent modifications to the individual axes of alignment between the virtual image and the second image.

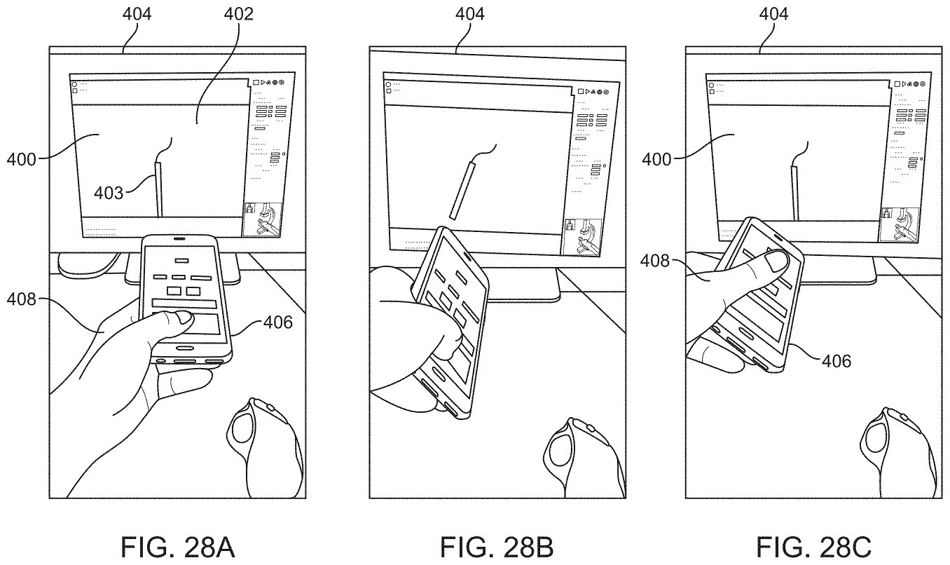

Referring now to FIGS. 28A-28C, manipulation of an image 400 of a 3D workspace 402 and/or a catheter 403 shown on a 2D display 404 using a 6 DOF input device 406 can be understood. Note that the view of the virtual workspace may optionally be driven here without inducing any actual changes in the position or shape of the virtual or actual camera, for example, to allow the user to see the shape of the catheter from a different orientation, or to see a position of the catheter relative to a nearby tissue along a different view axis, or to more clearly see a 2D planar image within a hybrid workspace, or the like. In this example, the system is in a springback mode that allows driving of the view to new positions and orientations, and that returns or springs the view back to the initial position after the drive command has ended. In other modes, the view remains in the position and orientation at the end of the view movement allowing a series of incremental view changes.

Referring now to FIG. 28A, prior to initiation of movement of the view the hand 408 of the user moves the input device 406 into a convenient position and orientation relative to the image of the catheter or other structure shown in the display. The user can initiate a movement command by actuating a drive view input button of the input device, and while the drive view button remains engaged, can move the input device 406 relative to the display 404 (see FIG. 28B). The image 400 shown on display 404 preferably changes in position and orientation in correlation with the movement of the input device 406, giving the user the impression of grasping the virtual and/or hybrid scene in the display and changing the users line of sight without inducing movement of the catheter or other structures seen in the display, and optionally without movement of any image capture device(s) providing any 2D or 3D image data included in image 400. As seen in FIG. 28C, when the hand 408 releases the drive view button of input device 406 the view orientation of the image shown in the display 404 returns back to its position at the start of the movement, with the speed of this spring back preferably being moderate to avoid user disorientation.

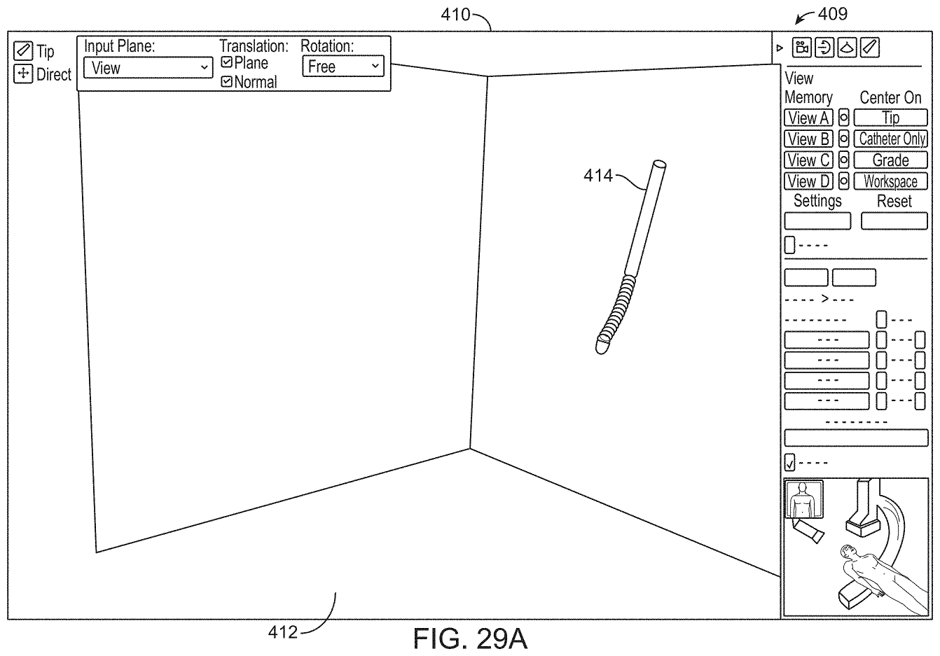

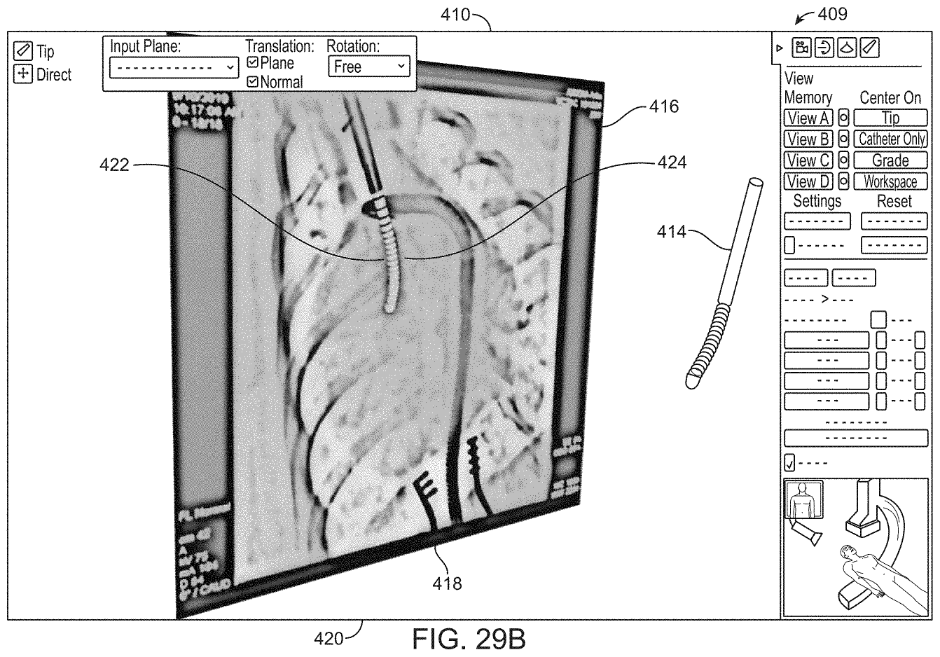

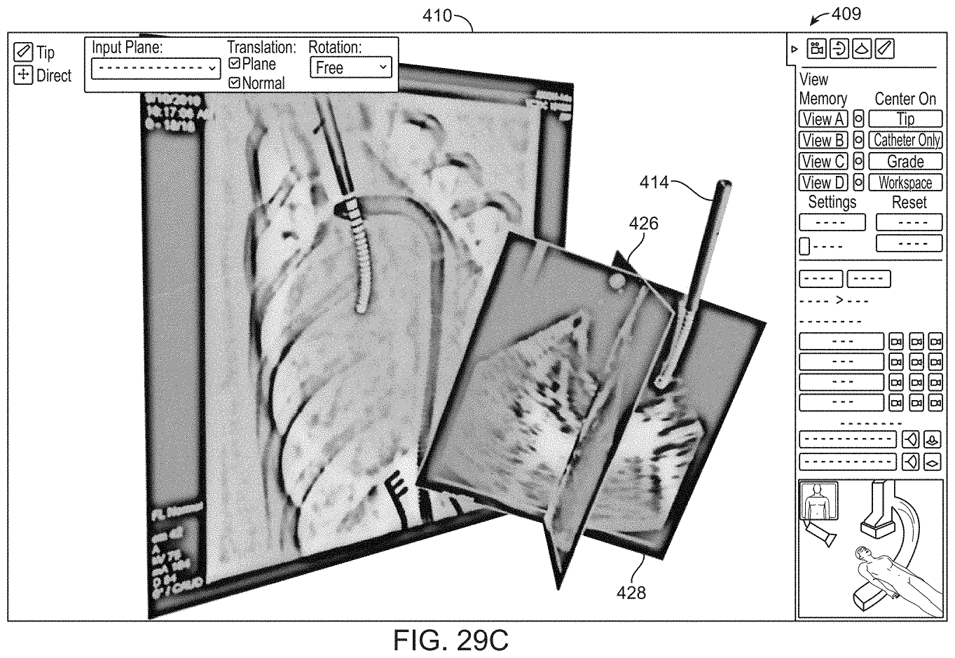

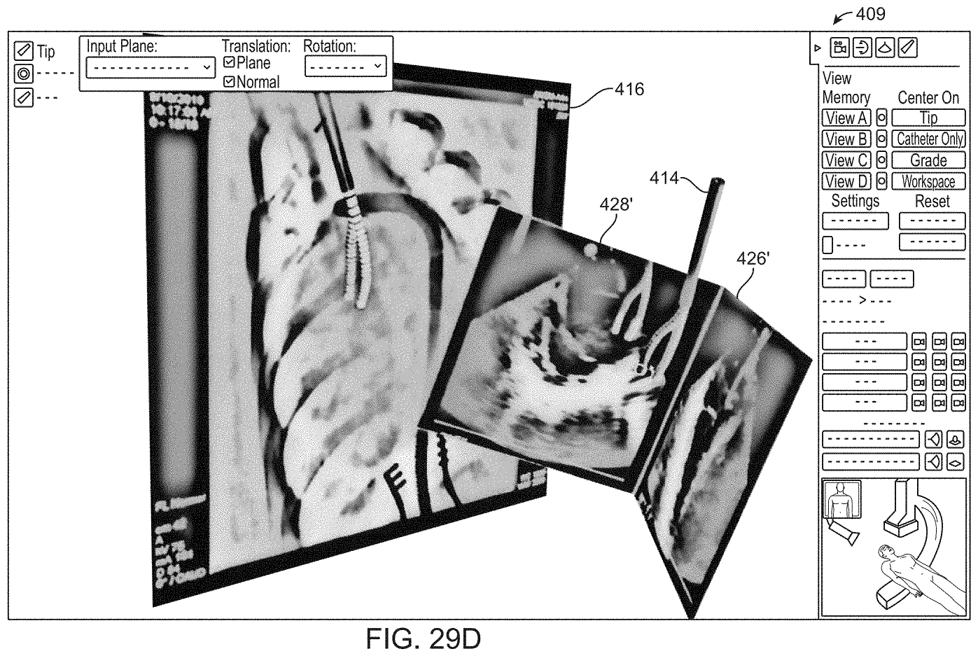

Referring now to FIGS. 29A-29D, components described above may be included in a hybrid 2D/3D image to be presented to a system user on a display 410, with the image components generally being presented in a virtual 3D workspace 412 that corresponds to an actual therapeutic workspace within a patient body. A 3D virtual image of a catheter 414 defines a pose in workspace 412, with the shape of the catheter often being determined in response to pressure and/or other drive signals of the robotic system, in response to imaging, electromagnetic, or other sensor signals so that the catheter image corresponds to an actual shape of an actual catheter. Similarly, a position and orientation of the 3D catheter image 414 in 3D workspace 412 corresponds to an actual catheter based on drive and/or feedback signals.

Referring still to FIGS. 29A-29D, additional elements which may optional be included in image 409 such as a 2D fluoroscopic image 416, the fluro image having an image plane 418 which may be shown at an offset angle relative to a display plane 420 of image 410 so that the fluoro image and the 3D virtual image of catheter 414 correspond in the 3D workspace. Fluoro image 416 may include an actual image 422 of an actual catheter in the patient, as well as images of adjacent tissues and structures (including surgical tools). A virtual 2D image 424 of 3D virtual catheter 414 may be projected onto the fluoro image 416 as described above. As seen in FIG. 29C, transverse or X-plane planar echo images 426, 428 may similarly be included in hybrid image 409 at the appropriate angles and locations relative to the virtual 3D catheter 414, with 2D virtual images optionally being projected thereon. However, as shown in FIG. 29D, it will often be advantageous to offset the echo image planes from the virtual catheter to generate associated offset echo images 426′, 428′ that can more easily be seen and referenced while driving the actual catheter. The planar fluoro and echo images within the hybrid image 409 will preferably comprise streaming live actual video obtained from the patient when the catheter is being driven.

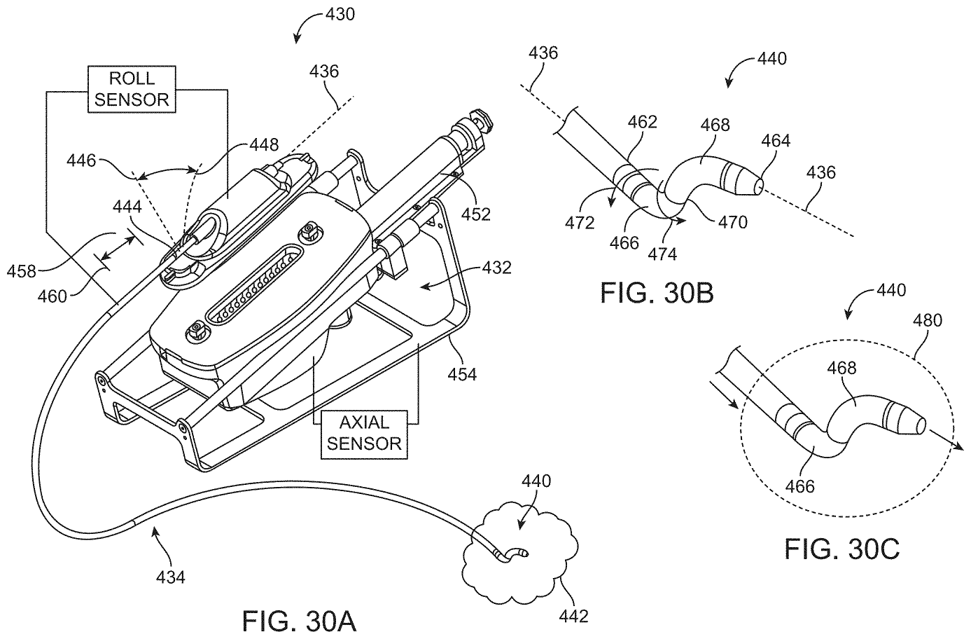

Referring now to FIGS. 30A-30C the proximal catheter housing and/or driver support structures will optionally be configured to both allow and sense manual manipulation of the catheter body outside the patient, and to drive the articulating tip in response to such manipulations so as to inhibit changes in position of the tip. More specifically, a catheter system 430 includes many of the components described above, including a driver 432 detachably receiving a catheter 434 having a flexible catheter body extending along an axis 436. A passive or un-driven proximal catheter body 438 extends distally to an actively driven portion 440 configured for use in an internal surgical site 442 within a patient body. A rotational handle 444 adjacent a proximal housing of the catheter allows the catheter body to be rotated relative to the driver about the catheter axis from a first rotational orientation 446 to a second rotational orientation 448, with the rotation being sensed by a roll sensor 450. An axial adjustment mechanism 452 couples the driver 432 to a driver support 545, and an axial sensor 456 senses changes in axial position of the catheter body when the mechanism is actuated manually by the user, for example to move between a first axial location 458 and a second axial location 460. Resulting rotation and/or axial translation of the catheter body induces corresponding rotation and/or translation at an interface 462 between the passive catheter body and the actively driven portion.

Referring now to FIGS. 30B and 30C, the articulated distal portion of the catheter can be articulated in response to the sensed rotational and/or axial movement so as to compensate for the movement of the interface 462 such that displacement of a distal tip 464 of the catheter within the patient in response to the movement of the interface is inhibited. Addressing roll about the catheter axis with reference to FIG. 30B, the articulated distal portion can include a proximal articulated segment 466 having a drive-alterable proximal curvature of axis 436 and a distal articulated segment 468 having a distal drive-alterable curvature of axis 436 with a segment interface 470 therebetween. When the manipulating of the proximal end of the catheter includes manually rotating the proximal end of the catheter about the axis of the catheter the articulating of the articulated distal portion can be performed so as to induce processing 472 of the proximal curvature about the axis of the catheter adjacent the interface, optionally along with processing 474 of the distal curvature about the axis of the catheter adjacent the segment interface such that lateral displacement of the distal tip of the catheter in response to the manual rotation of the catheter is inhibited. Manual rotation from outside the body with a fixed catheter tip inside the body can be particularly helpful for rotation of a tool supported adjacent the tip into a desired orientation about the axis of the catheter relative to a target tissue. Addressing manual movement along the catheter axis with reference to FIG. 30C, the articulated distal portion can similarly include a proximal articulated segment having a proximal curvature and a distal articulated segment having a distal curvature with a segment interface therebetween (see FIG. 30B). The articulating of the articulated distal portion can be performed so as to induce a first change in the proximal curvature and a second change in the distal curvature such that axial displacement of the distal tip of the catheter in response to the manual displacement of the catheter is inhibited, which can be useful for re-positioning a workspace 480 of a tool adjacent the distal tip of the catheter so as to encompass a target tissue.

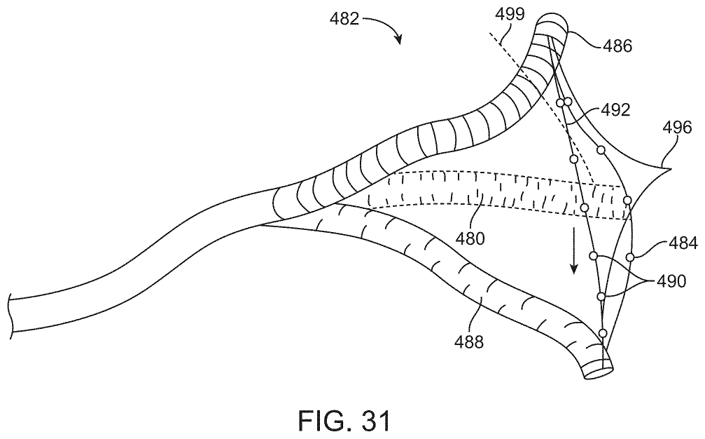

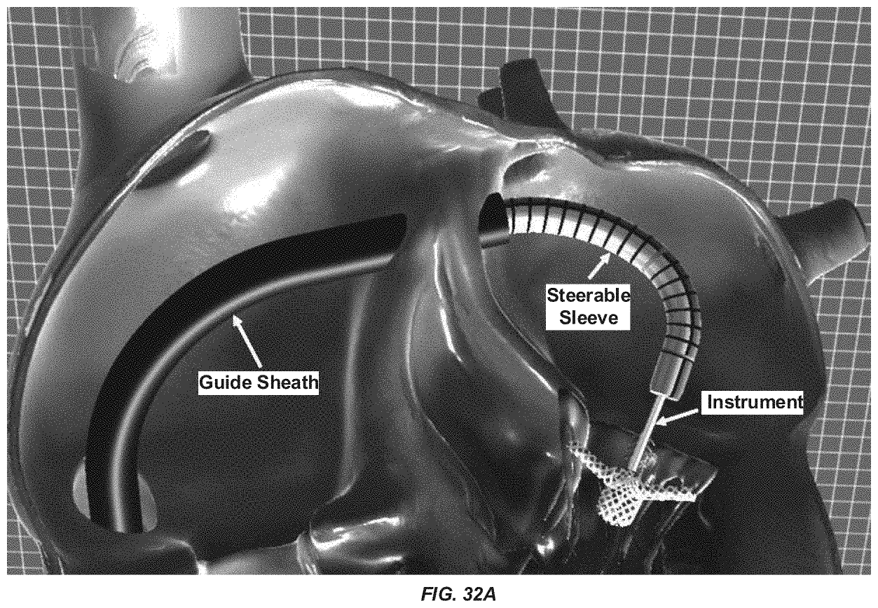

Referring now to FIG. 31, a virtual trajectory verification image 480 of the catheter can be included in the virtual and/or hybrid workspace image 482 to allow a user to visually review a proposed movement of the actual catheter along a trajectory 484 from a current catheter image 486 to a desired or phantom catheter image 488. To generate the trajectory 484, the processor may identify a plurality of verification locations 490 along an initial candidate trajectory 492 (such as a straight-line trajectory). The processor may seek to calculate drive signals for the verification locations using the methods described above, and for any of the verification locations outside a workspace boundary 494 of the catheter, the processor can identify alternative verification locations within the workspace. Smoothing the initial alternative path 496 between the alternative verifications locations can help provide a more desirable smoothed path to be used as the trajectory 484. Optionally, the current and desired locations may be identified in response to receipt, by the processor, of a command to go back to a prior pose of the catheter, with the desired pose comprising the prior pose and the catheter having moved from the prior pose along a previous trajectory.

From the disclosure above, it can be seen that robotic catheter system 10 (see FIG. 1) can be used to align a tool or receptacle 109 (see FIG. 5) with a target tissue (such as a heart valve of heart H) adjacent a workspace (such as a chamber of the heart) within a patient body P. The method may involve advancing an elongate structure such as catheter 12 axially along an access path within the patient body, the access path optionally being defined by a guide catheter 502 (see FIG. 11A), blood vessel, trans-septal access site, or the like. A first segment 504 of the elongate structure may be configured to laterally bend in response to a first segment drive command. The advancing of the catheter can be performed so that a first portion 504a of the first segment 504 is laterally unconstrained within the workspace and a second portion 504b of the first segment is laterally constrained along the access path.

A robotic controller such as processor 214 (see FIG. 19) can determine the first segment drive command from a desired tool position and/or desired pose 488 (see FIG. 31) and a first portion axial length of the first portion 504a. The robotic controller can transmit the first segment drive command so that the unconstrained first portion 504a of the first segment 504 bends laterally and the tool moves toward the desired tool position. The desired tool position can be defined by a desired tool path or trajectory 506, and the advancing, determining, and transmitting steps can be repeated so that the tool advances along the tool path.

The tool path 506 can be set by determining an aligned pose of the elongate structure in which the tool 109 is aligned with the target tissue. The elongate structure may have an axis 508 and the path may extend along the axis of the elongate structure in the aligned pose 488. The aligned pose can be determined by driving a virtual model 488, 302 (see FIGS. 25 and 31) of the elongate structure with reference to a display D showing an image of the target tissue, the driving of the virtual model typically including modeled lateral bending of the first segment and modeled axial advancement of the elongate structure, and by superimposing images of the virtual model of the elongate structure in the desired pose on the image of the target tissue in the display D. A candidate path 496 of the elongate structure along the axis 508 of the elongate structure in the aligned pose may be validated by superimposing a virtual validation model 480 of the elongate structure on the image of the target tissue in the display and moving the virtual validation model along the candidate path 496.

The advancing of the elongate structure can be measured with an axial sensor (see FIG. 30A), the axial sensor generating axial sensor signals and the controller 214 determining the segment drive commands in response to the axial signals. The axial advancing can be performed manually such as by manually actuating a handle of axial adjustment mechanism 452, or may be automated with a motor of the axial adjustment mechanism. As noted above with reference to FIG. 11A, when the access path comprises a lumen of an outer sheath having a lateral bending stiffness, the first segment drive signal can be determined using the lateral bending stiffness of the outer sheath or catheter 502. When the constraint is imposed by engagement between the advancing catheter and/or sheath and the surrounding tissue, the bending stiffness of the system may be calculated from measurements of lateral deflections along with bending torques and associated forces applied robotically by the catheter. As is also made clear with reference to FIG. 11A, a second segment 512 of the elongate structure can be configured to laterally bend in response to a second segment drive command, the second segment being axially offset (proximally or distally) from the first segment. When the first segment is proximal of the second segment, the robotic controller can derive the second segment drive command using the desired tool position and the first portion axial length so that the first and second drive commands together move the tool toward the desired tool position. When the first segment is distal of the second segment the robotic controller can determine the first segment drive command using the lateral bending of the second segment as constrained by the outer sheath 502. As described above with reference to FIG. 19, driving of model, virtual, and/or validation catheters will often be performed using simulation module 256 of robotic processor 214.

Limitations with a single consistent 6/7 DOF inverse kinematic (IK) solver

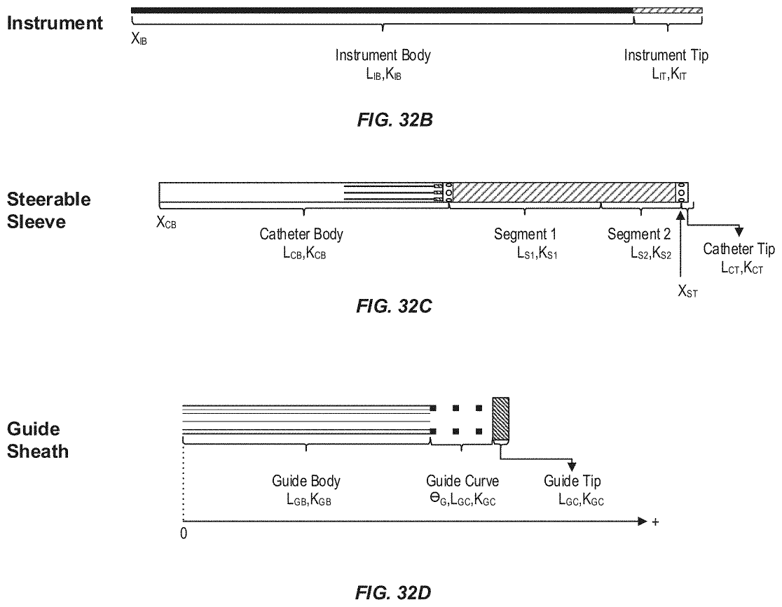

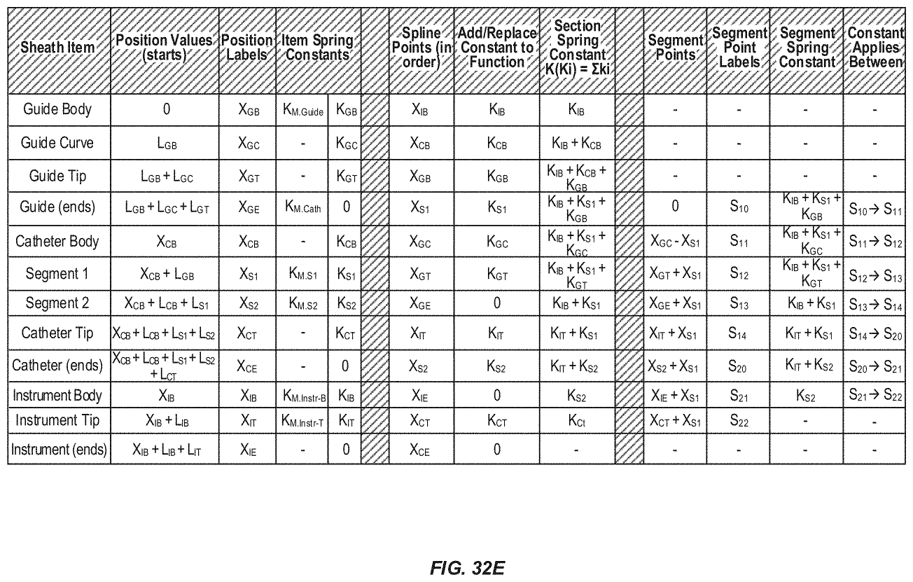

With reference to FIGS. 32A-32E, the two-segment articulated bodies described herein will often have 6 degrees of freedom (DOF) where each segment carries 3 DOF, one in extension and two in bend. During insertion, when the segments pass through a rigid or semi-rigid Guide sheath, the portion of the segments within the Guide has limited or no bend.

Artificially reducing the DOF

Mapping (or reducing) the IK solvers DOF to better match the available DOF of the Steerable Sleeve as it emerges out of the Guide sheath should improve the stability of the IK solver and AR modeling accuracy of the physical segments. The following conditions can optionally be assumed for determining which DOF might be removed:
When a segment is fully within the Guide sheath it essentially loses the bend DOF and is limited to only elongation.
When a segment is partially out of the Guide sheath the bend DOFs return and but can be unstable (observably at segment stiffness change transitions) with the segment's extension DOF.
When a segment is fully out of the Guide sheath the bend and elongation DOF's tend to be stable.
Segments fully out of the Guide sheath do not cause instability to a proximal segment in transition through the Guide sheath.

Mapping DOF

Table 25 lists the control type and IK DOF at different Guide sheath emersion states.

TABLE 25

| Emersion | | Distal | Prox. | Prox. | |
|---|---|---|---|---|---|
| Configu- | Segments | Segment | Segment | Segment | Segments |
| ration | Fully In | Emerges | Emerges | Emerges | Fully Out |
| Segment 1 | In (Guide) | In | In/Out | In/Out | Out |
| (proximal) | | | (or Out) | (or Out) | |
| Segment 2 | In (Guide) | In/Out | Out | Out | Out |
| (distal) | | | | | |
| Direct IK | 0 DOF | 2 DOF | 4 DOF | 5 DOF | 6 DOF |
| Solver | | | | | |
| Phantom IK | 0 DOF + | 2 DOF + | 4 DOF + | 5 DOF + | 6 DOF + |
| Solver | 1 Axial | 1 Axial | 1 Axial | 1 Axial | 1 Axial |
| S1A, S1B, | Pr | Pr | IK (2D | IK (2D | IK |
| S1C | | | Bend) | Bend) | |
| S2A, S2B, | Pr | IK (2D | IK (2D | IK | IK |
| S2C | | Bend) | Bend) | | |

Pr = Direct Pressure Control
IK = Inverse Kinematics Control
2D Bend = 2 DOF are for segment bend

Five IK Solver Types

Table 25 above indicates five controller configurations for both the Direct and Phantom IK solvers. The Phantom controller incorporates the same segment controls as the Direct but adds a Catheter Body insertion DOF. Only the Direct IK solvers will be described as the Phantom follows suit.

The Cortex optionally uses the 6 DOF IK controller which drives segments through every emersion configuration in the table above. Note this is highlighted in yellow in the table.

To create the reduced DOF IK solvers, several methods are implemented, the first which is to separately control the lumen channels by direct pressure control. The direct pressure controllers regulate the lumen to a specific pressure. The 2 DOF IK solver, used on the distal segment as it emerges, allows three lumen pressures in the segment to change while maintaining the sum of all three to a constant value, which fixes the segment length. The 4 DOF IK solver is applied on both segments (once the proximal segment has at least partially emerged) constrains the pressure in each segment to maintaining a fixed segment length. The 5 DOF IK solver similar constrains the pressure in the proximal segment as it emerges and incorporates the full 3 DOF of the distal segment into the controller.

The analysis below applies nomenclature which can be understood with reference to FIGS. 11A and 32A-G:
Segments Fully In (the Guide Sheath): Proximal and distal segments lengths adjusted through direct pressure control with lengths adjusted as a function of distance to the end of the Guide Sheath. Each Segment's pressures transition independently from the minimum pressure to a middle pressure (halfway between minimum and maximum) as the distal end of the segment meets the distal end of the Guide Sheath. The pressure relationships between the three lumens of each segment are as follows:

Pr_{1A}=Pr_{1B}=Pr_{1C}=Pr_{1_SET} where Pr_{MIN}<=Pr_{1_SET}<=Pr_{MID}

Pr_{2A}=Pr_{2B}=Pr_{2C}=Pr_{2_SET} where Pr_{MIN}<=Pr_{2_SET}<=Pr_{MID}

Distal Segment Emerges (from Guide Sheath): A hybrid of the Direct Pressure Control for the proximal segment and a 2 DOF IK solver control for the distal segment is combined. For the latter, two of the pressure variables are used in the IK Solver and the third is defined. The control relationship between segments and within each is as follows:

Pr_{1A}=Pr_{1B}=Pr_{1C}=Pr_{1_MID}

Pr_{2A}+Pr_{2B}+Pr_{2C}=3*Pr_{MID}

A direct formula is available for both forward kinematics (FK) and inverse kinematics (IK) from the desired position as follows:

P=(Pₓ,P_{y},P_{z}) where P is the command point

Alpha=ATAN2(P_{y},Pₓ)

Beta=2*A SIN(P×V) where P×V=_{[}(pₓ²+P_{y}²)/(Pₓ²+P_{y}²+P_{z}²)]^{1/2}

S is determined by the length extended out of the Guide sheath.

Proximal Segment Emerges (from Guide Sheath)—Fixed Distal Length: The 4 DOF IK solver controls all segments and lumen pressures while only two pressure variables are used from both segments with its third defined. The proximal lumen pressures are set where two lumens are input variables and one is set. There is no direct pressure control. The control relationship is as follows:

Pr_{1A}+Pr_{1B}+Pr_{1C}=3*Pr_{1_MID}

Pr_{2A}+Pr_{2B}+Pr_{2C}=3*Pr_{2_MID}

Use the forward kinematics for the 6 DOF system where Pr_{1C}=3*Pr_{1_MID}−Pr_{1A}−Pr_{1B} and Pr_{2C}=3*Pr_{2_MID}−Pr_{2A}−Pr_{2B}. Pr_{1A}, Pr_{1B}, Pr_{2A}, and Pr_{2B} are the input variables and Pr_{1C} and Pr_{2C} are adjusted to maintain the constant length requirement. Note that any two of the Pr₁ and Pr₂ terms can be used as the IK solver variables as long as the remaining Pr's are set by using the constant length formula listed above.

Proximal Segment Emerges (from Guide Sheath): The 5 DOF IK solver controls all segments and lumen pressures while only two pressure variables are used from the proximal segment with the third defined. The proximal lumen pressures are set where two lumens are input variables and one is set. There is no direct pressure control. The control relationship is as follows:

Pr_{1A}+Pr_{1B}+Pr_{1C}=3*Pr_{MID}

Use the forward kinematics for the 6 DOF system where Pr_{1C}=3*Pr_{MID}−Pr_{1A}−Pr_{1B}. Pr_{1A} and Pr_{1B} are the input variables and Pr_{1C} is adjusted to maintain the constant length requirement. Note that any two of the Pr₁ terms can be used as the IK solver variables as long as the remaining one is set by using the constant length formula listed above.

Segments Fully Out (of Guide Sheath): All pressure channels are regulated directly by the 6 DOF IK Solver. There is no direct pressure control and no setting of pressures by other lumen pressures.

Merging Transitions

While each control type is stable in isolation, transitioning between them benefits from consideration. An abrupt transition from one control type to the next may cause a step change in the tip positioning and orientation. When teetering between two types of IK solutions, movement between them could oscillate and cause instability. A linear transition that shifts control from one IK solution to the next, while covering a span of axial displacement, may be employed to avoid disruptive transitions. A methodology is described as follows.

IK Transitions: During a segment's emersion, start the IK transition as the distal end of a segment protrudes through the Guide Sheath and extends. The distance could be 5, 10, or 20 percent the length of the segment, or could be measured in units of length such as 1, 2 or 4 millimeters. During retraction, the transition would start at the set distance distally from the Guide Sheath. A transition equation is as follows:

Pr_{TR}=(X/X_{TR})*Pr(IK₁)+[(X_{TR}−X)/X_{TR}]*Pr(IK₂)
Where 0<=X<X_{TR}
   X=Length between Guide and protruding Segments (distal ends)
   X_{S}=Transition length
   IK₁=Proximal IK solver
   IK₂=Distal IK solver

The following section is for reference and includes formulas used for calculation between various known and unknown segment properties.

Segment DOF FK Equations

For a three-balloon array segment has the following solutions between lumen pressures (Pr_{A}+Pr_{B}+Pr_{C}), side lengths (centered on the balloon array S_{A}, S_{B}, S_{C}), and joint variables (S_{Calc}, βx, βy):

Lumen pressures from joint variables

Pr_{A}=(S_{Calc}*K_{F}+F₀)/(3*A)−2*βₓ*K_{M}/(3*A*r_{A})

Pr_{B}=(S_{Calc}*K_{F}+F₀)/(3*A)+(βₓ−3*β_{y}/3^{1/2})*K_{M}/(3*A*r_{A})

Pr_{C}=(S_{Calc}*K_{F}+F₀)/(3*A)+(βₓ+3*β_{y}/3^{1/2})*K_{M}/(3*A*r_{A})

Lumen Pressures from Segment Lengths (using Spring pressure math)

Pr_{A}=(1/3)*[(1/3)*K_{F}*(S_{A}+S_{B}+S_{C})+F₀]/A+(1/3)*(2/3)*[K_{M}/(A*r_{A}²)]*(2*S_{A}−S_{B}−S_{C})

Pr_{B}=(1/3)*[(1/3)*K_{F}*(S_{A}+S_{B}+S_{C})+F₀]/A+(1/3)*(2/3)*[K_{M}/(A*r_{A}²)]*(2*S_{A}−S_{C}−S_{A})

Pr_{C}=(1/3)*[(1/3)*K_{F}*(S_{A}+S_{B}+S_{C})+F₀]/A+(1/3)*(2/3)*[K_{M}/(A*r_{A}²)]*(2*S_{C}−S_{A}−S_{B})

Segment Lengths from joint variables

S_{A}=S_{Calc}−r_{A}*βₓ

S_{B}=S_{Calc}+(1/2)*r_{A}*βₓ−(1/2)*(3/3^{1/2})*r_{A}*β_{y}

S_{C}=S_{Calc}+(1/2)*r_{A}*βₓ+(1/2)*(3/3^{1/2})*r_{A}*β_{y}

Segment Lengths from Lumen Pressure

S_{A}=[A*(Pr_{A}+Pr_{B}+Pr_{C})−F₀]/K_{F}+(1/2)*A*r_{A}²*(2*Pr_{A}−Pr_{B}−Pr_{C})/K_{M}

S_{B}=[A*(Pr_{A}+Pr_{B}+Pr_{C})−F₀]/K_{F}+(1/2)*A*r_{A}²*(2*Pr_{B}−Pr_{C}−Pr_{A})/K_{M}

S_{C}=[A*(Pr_{A}+Pr_{B}+Pr_{C})−F₀]/K_{F}+(1/2)*A*r_{A}²*(2*Pr_{C}−Pr_{A}−Pr_{B})/K_{M}

Joint variables from Segment Lengths

S_{Calc}=[S_{A}+S_{B}+S_{C})/3

βₓ=(1/3)*(S_{C}+S_{B}−2*S_{A})/r_{A}=

β_{y}=(3^{1/2}/3)*(S_{C}−S_{B})/r_{A}

Joint variables from lumen pressures

S_{Calc}=[A*(Pr_{A}+Pr_{B}+Pr_{C})−F₀]/K_{F}

βₓ=(1/2)*A*r_{A}*(Pr_{C}+Pr_{B}−2*Pr_{A})/K_{M}

β_{y}=(3^{1/2}/2)*A*r_{A}*(Pr_{C}−Pr_{B})/K_{M}

The identified positions along the articulating catheter system can be used to define splines. When such positions are generated from a virtual or phantom articulating catheter model that is manipulated by the user within the workspace, these splines can be used to calculate and define a trajectory along which the robot can be inserted, optionally by robotically driving lateral articulation during manual insertion of the catheter into the patient, robotically driving lateral articulation during powered insertion of the catheter, or the like.

While the exemplary embodiments have been described in some detail for clarity of understanding and by way of example, a variety of modifications, changes, and adaptations of the structures and methods described herein will be obvious to those of skill in the art. Hence, the scope of the present invention is limited solely by the claims attached hereto.

What is claimed is:

1. A method for aligning a tool with a target tissue adjacent a workspace within a patient body, the method comprising:
advancing an elongate structure axially along an access path within the patient body, a first segment of the elongate structure being configured to laterally bend in response to a first segment drive command, wherein the advancing is performed so that a first portion of the first segment is laterally unconstrained within the workspace and a second portion of the first segment is laterally constrained along the access path;
determining, with a robotic controller, the first segment drive command from a desired tool position and a first portion axial length of the first portion; and
transmitting the first segment drive command from robotic controller so that the unconstrained first portion of the first segment bends laterally and the tool moves toward the desired tool position.

2. The method of claim 1, wherein the desired tool position is defined by a desired tool path, and wherein the advancing, determining, and transmitting steps are repeated so that the tool advances along the tool path.

3. The method of claim 2, further comprising defining the path by determining an aligned pose of the elongate structure with the tool aligned with the target tissue, wherein the elongate structure has an axis and the path extends along the axis of the elongate structure in the aligned pose.

4. The method of claim 3, wherein the aligned pose is determined by driving a virtual model of the elongate structure with reference to a display showing an image of the target tissue, the driving of the virtual model including modeled lateral bending of the first segment and modeled axial advancement of the elongate structure, and superimposing images of the virtual model of the elongate structure on the image of the target tissue in the display.

5. The method of claim 4, further comprising validating a candidate path of the elongate structure along the axis of the elongate structure in the aligned pose by superimposing a virtual validation model of the elongate structure on the image of the target tissue in the display and moving the virtual validation model along the candidate path.

6. The method of claim 4, wherein the advancing of the elongate structure is measured with an axial sensor, the axial sensor generating axial sensor signals and the controller determining the first segment drive commands in response to the axial signals.

7. The method of claim 6, wherein the axial advancing is performed manually.

8. The method of claim 1, wherein the access path comprises a lumen of an outer sheath having a lateral bending stiffness, and wherein the first segment drive signal is determined using the lateral bending stiffness of outer sheath.

9. The method of claim 1, wherein a second segment of the elongate structure is configured to laterally bend in response to a second segment drive command, the second segment being axially offset from the first segment.

10. The method of claim 9, wherein the first segment is proximal of the second segment, and further comprising determining, with the robotic controller, the second segment drive command from the desired tool position so that the first and second drive commands together move the tool toward the desired tool position.

11. The method of claim 10, wherein the robotic controller determines the first and second segment drive commands with a first number of command degrees of freedom, and further comprising determining a constrained drive command with a second number of command degrees of freedom smaller than the first number when at least a portion of the first segment is constrained.

12. The method of claim 9, wherein the first segment is distal of the second segment, and further comprising determining, with the robotic controller, the first segment drive command in response to constrained lateral bending of the second segment.

13. A method for aligning a tool with a target tissue adjacent a workspace within a patient body, the method comprising:
driving, with reference to a display, a virtual model of an elongate structure from an actual pose of the elongate structure to a desired pose, the display showing an image of the elongate structure, an image of the target tissue, and a superimposed image of the virtual model, the elongate structure having an axis; and
advancing the elongate structure axially along a desired path within the patient body by transmitting drive commands to laterally bend the elongate body, the desired path being defined by the axis of the virtual model of the elongate body in the desired pose.

14. A method for aligning a tool with a target adjacent a workspace, the method comprising:
driving a continuum robot per a first drive signal in response to a first command to move a distal end of the continuum robot toward the target, wherein the continuum robot has proximal and distal articulatable portions, and wherein the first signal is determined with a first number of degrees of freedom by a robotic controller in response to the proximal portion being laterally constrained; and
driving the continuum robot per a second drive signal in response to a second command to move the distal end toward the target, wherein the second signal is determined with a second number of degrees of freedom by the robotic controller in response to the proximal portion being laterally unconstrained, the second number being larger than the first number.

15. A system for aligning a tool with a target tissue adjacent a workspace within a patient body, the system comprising:
an elongate structure having a proximal end and a distal end with an axis therebetween, the elongate structure configured to advance axially along an access path within the patient body, a first segment of the elongate structure having a first segment length and being configured to laterally bend in response to a first segment drive command; and
a robotic controller coupled to the first segment, the robotic controller configured to generate the first segment drive command from a desired tool position and a laterally constrained axial portion length so that the unconstrained first portion of the first segment bends laterally and the tool moves toward the desired tool position.

16. A system for aligning a tool with a target tissue adjacent a workspace within a patient body, the system comprising:
an elongate structure having a proximal end and a distal end with an axis therebetween, the elongate structure configured to advance axially into a patient body;
a simulation system configured to drive, with reference to a display, a virtual model of the elongate structure from an actual pose of the elongate structure to a desired pose, the display, in use, showing an image of the elongate structure and an image of the target tissue, the simulation system configured to superimpose an image of the virtual model on the display; and
a robotic controller coupled with the elongate structure and configured to help advance the elongate structure axially along a desired path within the patient body by transmitting drive commands to laterally bend the elongate body, the desired path being defined by the axis of the virtual model of the elongate body in the desired pose.

17. A system for aligning a tool with a target adjacent a workspace, the system comprising:
a continuum robot having a proximal end and a distal end with an axis therebetween, the continuum robot having axially offset proximal and distal articulatable portions and configured to advance axially from a lateral constraint into the workspace;
a robotic controller coupled with the continuum robot, the controller having a first mode for use when the proximal portion is laterally constrained for transmitting to the continuum robot a first drive signal in response to a first command to move the distal end toward the target, the first signal having a first number of degrees of freedom; and
the robotic controller having a second mode when the proximal portion is unconstrained for driving the continuum robot per a second drive signal in response to a second command to move the distal end toward the target, wherein the second signal has a second number of degrees of freedom, the second number being larger than the first number.
